(19) United States
(12) Patent Application Publication
(10) Pub. No.: US 2024/0415914 A1
(43) Pub. Date: Dec. 19, 2024

(54) Water Dispersible Cannabinoid Compositions

(71) Applicant: Aquila Black Limited, North Perth (AU)

(72) Inventor: Cameron Scadding, North Perth (AU)

(21) Appl. No.: 18/705,885

(22) PCT Filed: Oct. 31, 2022
(86) PCT No.: PCT/AU2022/051307

§ 371 Date: Apr. 29, 2024

(30) Foreign Application Priority Data
Oct. 29, 2021 (AU) 2021903467

(51) Int. Cl. A61K 36/185; A23L 2/39; A23L 2/52; A61K 9/00; A61K 9/107; A61K 31/00; A61K 47/02; A61K 47/10; A61K 47/12; A61K 47/22; A61K 47/36; A61K 47/44

(52) U.S. Cl. CPC A61K 36/3482; A23L 2/39; A23L 2/52; A61K 9/0095; A61K 9/1075; A61K 31/658; A61K 47/02; A61K 47/10; A61K 47/12; A61K 47/22; A61K 47/36; A61K 47/44

(57) ABSTRACT

Water dispersible cannabinoid compositions suitable for the preparation of beverages, in particular beverages containing one or more cannabinoids, beverage concentrates and methods of preparing same.

### TECHNICAL FIELD

The disclosure relates to water dispersible cannabinoid compositions suitable for the preparation of beverages, in particular beverages containing one or more cannabinoids, beverage concentrates and methods of preparing same.

This disclosure outlines the preparation of compositions comprising cannabinoids that can be used in other applications, including but not limited to applications involving the preparation of solid foods comprising cannabinoids, and/or applications in the preparation of medicaments or pharmaceutical formulations comprising cannabinoids.

### BACKGROUND

The discussion of the background to the disclosure is intended to facilitate an understanding of the disclosure. However, it should be appreciated that the discussion is not an acknowledgement or admission that any of the material referred to was published, known or part of the common general knowledge as at the priority date of the application.

The consumption of alcohol is a well-entrenched social convention, and it is common for people to consume alcohol to relax after work or regard it as an accepted part of celebrations and social occasions with family and friends.

There is growing concern, however, about alcohol-related health issues. In particular, there is convincing evidence that drinking even small amounts of alcohol increases the risk of cancers of the bowel, breast, mouth, throat, larynx, oesophagus and liver.

Consequently, popular campaigns such as “Dry July” or “Go Sober for October” have been established to encourage people not to consume alcohol for a limited period of time with the objective of moderating alcohol consumption habits in the long term. Additionally, alcoholic beverage makers are adapting to emerging market trends and introducing low alcohol beers or non-alcoholic beverages.

Alcohol consumption trends indicate that while personal health/lifestyle factors and a focus on moderation are influencing a reduction in alcohol consumption, the main reasons for alcohol consumption, such as social reasons and stress reduction remain as strong. There is a need, therefore, to provide a beverage which could be used by the consumer to relax and unwind but which does not have the health risks known to be associated with alcohol consumption.

The use of oils and extracts obtained from cannabis plant material for medical purposes is becoming more common. Pharmacologically active chemicals found in cannabis plant material, such as delta-9-tetrahydrocannabinol (or THC) and cannabidiol (CBD), have been proven to decrease the symptoms of nausea and vomiting induced by chemotherapy treatments. Cannabinoids and other compounds found in cannabis have also been shown to stimulate bone growth, relieve pain, aid sleep, inhibit bacterial cell growth, inhibit cancer cell growth, and alleviate or otherwise reduce the symptoms of cancer, epilepsy, autoimmune disease, neurodegeneration, Lyme disease, post-traumatic stress disorder, inflammation and Alzheimer's disease, according to a rapidly growing body of research. Furthermore, extracts of cannabis plant material have been found to provide therapeutic benefits in individuals with glaucoma, dysmenorrhea, migraines, anxiety disorders, or a combination of these conditions.

However, cannabis plant material or cannabis oil is commonly smoked or inhaled via delivery devices such as vaporizers and E-cigarettes. Such oils, when vaporized or smoked, are often rough on a patient's throat and may induce coughing or gagging. Furthermore, such delivery methods substantially increase the risk of lung, throat and mouth cancers, as well as Chronic Obstructive Pulmonary Disease (COPD), heart disease and stroke, thereby negating many of the positive therapeutic benefits associated with cannabinoids. There is a need, therefore, to provide alternative methods of delivery for cannabinoids.

Cannabis-infused beverages have been proposed as viable alternatives to alcohol-based drinks and cannabis delivery methods involving inhalation, but there are several problems associated with delivering a shelf-stable cannabis-containing beverage. Firstly, cannabinoids are both heat and light sensitive and may degrade rapidly unless stored in dark containers under temperature-controlled conditions. Secondly, cannabinoids are highly lipophilic and therefore immiscible in water. Consequently, cannabis-containing beverages may have or develop an undesirable cloudy appearance and oily droplets containing cannabis extracts may coalesce to form films, leaving unsightly residues.

Existing approaches to solving this problem involve the use of ethanol to solubilise the lipophilic cannabinoids, such as the approach described in U.S. Pat. No. 9,642,884, which involves preparing cannabis containing beverages by dissolving milligram quantities of isolated cannabinoids in ethanol and adding the resulting solution to a consumable alcohol drink such as vodka. However, such approaches do not address the problem of providing an alcohol free beverage comprising cannabinoids.

Further problems arise from the fact that the beneficial effect of cannabinoids may be significantly delayed, in part, due to high lipophilicity, when ingested, which can lead the consumer to imbibe a greater volume than required, leading to a delayed and stronger reaction than originally intended. There is a need to develop ingestible cannabinoid compositions that minimise the delay in onset of effects and maximise the bioavailability of the cannabinoid compounds therein.

Finally, cannabinoids pose significant problems from the point of view establishing an effective global supply chain for the beverage industry. Shipping of large quantities of beverages is highly inefficient, particularly in view of the fact that the majority of a beverage is almost invariably water. Furthermore, the instability of cannabinoids and cannabis extracts due to oxidation and other mechanisms of degradation may be exacerbated when cannabinoid compounds are in solution. There is a need to develop stable, easily transportable, easily doseable, water dispersible concentrates comprising cannabinoids.

Various embodiments as disclosed herein seek to overcome at least some of the aforementioned disadvantages and look to enable the development of a global supply chain for cannabinoids as a stable ingredient.

### SUMMARY

The disclosure relates to water dispersible cannabinoid compositions suitable for the preparation of beverages, in particular beverages containing one or more cannabinoids, beverage concentrates and methods of preparing same.

This disclosure provides methods for preparing stable water dispersible concentrates comprising emulsions containing one or more cannabinoids.

This disclosure also provides methods for preparing stable water soluble powders containing one or more cannabinoids.

Various embodiments provide a beverage composition comprising a nanoemulsion dispersed in a drinkable liquid, wherein the nanoemulsion comprises one or more cannabinoids in an oil phase, at least one surfactant and an aqueous phase.

In preferred embodiments, the at least one surfactant does not comprise a surfactant selected from the group consisting of; polyoxyethylenated sorbitan monooleate (Tween-80), polyoxyethylenated sorbitan monostearate (Tween-60), polyoxyethylenated sorbitan monopalmitate (Tween-40), polyoxyethylenated sorbitan monolaurate (Tween-20), sorbitan monooleate (Span 80), polyethoxylated castor oil derivatives (including Kolliphor EL, Cremophor EL and other saturated or unsaturated polyethoxylated castor oil derivatives), ethoxylated fatty acids [including poly(ethylene glycol) monooleate, and Polyethylene glycol (15)-hydroxystearate (Solutol)], polyethoxylated hydrogenated fatty acid ethers [including Polyoxyethylene (10) oleyl ether (BRIJ97)], ethoxylated monoglycerol esters, soy lecithins (including ALCOLEC F-100), sucrose fatty acid esters, sucrose palmitates, and sucrose stearates.

In one embodiment the oil phase comprises an edible oil, a medium chain triglyceride, paraffin oil or any short chain triglyceride oil. For example, the oil phase may comprise an essential oil, such as, but not limited to orange oil.

In one embodiment, the one or more cannabinoids are the group consisting of; anandamide, 2-arachidonoylglycerol, cannabichromene (CBC), cannabichromenic acid (CBCA), cannabichromevarin (CBCV), cannabichromevarinic acid (CBCVA), cannabidiol (CBD), cannabidiolic acid (CBDA), cannabidivarin (CBDV), cannabidivarinic acid (CBDVA), cannabielsoin (CBE), cannabicyclol (CBL), cannabinodiol (CBND), cannabigerol (CBG), cannabigerolic acid (CBGA), cannabigerovarin (CBGV), cannabigerovarinic acid (CBGVA), cannabinol (CBN), cannabinolic acid (CBNA), cannabitriol (CBT), delta-8-tetrahydrocanninol, delta-8-tetrahydrocannabinolic acid, delta-9-tetrahydrocannabinol (THC), delta-9-tetrahydrocannabinolic acid (THCA), delta-9-tetrahydrocannabivarin (THCV), delta-9-tetrahydrocannabivarinic acid (THCVA), 11-nor-9-carboxy-delta-9-tetrahydrocannabinol (THCCOOCH), 11-nor-9-carboxy-delta-8-tetrahydrocannabinol, 11-hydroxy-delta-8-tetrahydrocannabinol, and 11-hydroxy-delta-9-tetrahydrocannabinol, dimethyl heptylpentyl cannabidiol (DMHP-CBD), 6,12-dihydro-6-hydroxy-cannabidiol, (3S,4R)-7-hydroxy-.DELTA.6-tetrahydrocannabinol homologs and derivatives, (+)-4-[4-DMH-2,6-diacetoxy-phenyl]-2-carboxy-6,6-dimethylbicyclo[3.1.1]hept-2-en, and other 4-phenylpinene derivatives, and cannabidiol (−)(CBD) analogs such as (−)CBD-monomethylether, (−)CBD dimethyl ether; (−)CBD diacetate; (−)3′-acetyl-CBD monoacetate, cannabinol propyl variant (CBNV), nabilone, synthetic cannabinoids, and cannabinoid analogues, including derivatives, analogues, stereoisomers, racemates, scalemates and pharmaceutically acceptable salts of any of the aforementioned cannabinoids.

In one embodiment, the one or more cannabinoids may be selected from the group comprising cannabis extracts, cannabis oils, and cannabis resins.

In some embodiments, the nanoemulsion and powder compositions of the invention may comprise an entourage of cannabinoids derived from a cannabis plant, cannabis extract, cannabis oil, or cannabis resin.

In some embodiments, the entourage of cannabinoids comprises cannabichromene (CBC), cannabidiol (CBD), cannabidiolic acid (CBDA), cannabidivarin (CBDV), cannabicyclol (CBL), cannabigerol (CBG), cannabinol (CBN), delta-9-tetrahydrocannabinol (THC), delta-9-tetrahydrocannabinolic acid (THCA), and delta-9-tetrahydrocannabivarin (THCV).

In certain embodiments, the cannabinoid may comprise THC. In certain embodiments, the cannabinoid may comprise CBD. In other embodiments, the cannabinoid may comprise THC and/or CBD.

In some embodiments, each cannabinoid is present in the water dispersible nanoemulsion compositions of the invention in an amount of approximately 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20, 20.1, 20.2, 20.3, 20.4, 20.5, 20.6, 20.7, 20.8, 20.9, 21, 21.1, 21.2, 21.3, 21.4, 21.5, 21.6, 21.7, 21.8, 21.9, 22, 22.1, 22.2, 22.3, 22.4, 22.5, 22.6, 22.7, 22.8, 22.9, 23, 23.1, 23.2, 23.3, 23.4, 23.5, 23.6, 23.7, 23.8, 23.9, 24, 24.1, 24.2, 24.3, 24.4, 24.5, 24.6, 24.7, 24.8, 24.9, 25, 25.1, 25.2, 25.3, 25.4, 25.5, 25.6, 25.7, 25.8, 25.9, 26, 26.1, 26.2, 26.3, 26.4, 26.5, 26.6, 26.7, 26.8, 26.9, 27, 27.1, 27.2, 27.3, 27.4, 27.5, 27.6, 27.7, 27.8, 27.9, 28, 28.1, 28.2, 28.3, 28.4, 28.5, 28.6, 28.7, 28.8, 28.9, 29, 29.1, 29.2, 29.3, 29.4, 29.5, 29.6, 29.7, 29.8, 29.9, 30, 30.1, 30.2, 30.3, 30.4, 30.5, 30.6, 30.7, 30.8, 30.9, 31, 31.1, 31.2, 31.3, 31.4, 31.5, 31.6, 31.7, 31.8, 31.9, 32, 32.1, 32.2, 32.3, 32.4, 32.5, 32.6, 32.7, 32.8, 32.9, 33, 33.1, 33.2, 33.3, 33.4, 33.5, 33.6, 33.7, 33.8, 33.9, 34, 34.1, 34.2, 34.3, 34.4, 34.5, 34.6, 34.7, 34.8, 34.9, 35, 35.1, 35.2, 35.3, 35.4, 35.5, 35.6, 35.7, 35.8, 35.9, 36, 36.1, 36.2, 36.3, 36.4, 36.5, 36.6, 36.7, 36.8, 36.9, 37, 37.1, 37.2, 37.3, 37.4, 37.5, 37.6, 37.7, 37.8, 37.9, 38, 38.1, 38.2, 38.3, 38.4, 38.5, 38.6, 38.7, 38.8, 38.9, 39, 39.1, 39.2, 39.3, 39.4, 39.5, 39.6, 39.7, 39.8, 39.9, 40, 40.1, 40.2, 40.3, 40.4, 40.5, 40.6, 40.7, 40.8, 40.9, 41, 41.1, 41.2, 41.3, 41.4, 41.5, 41.6, 41.7, 41.8, 41.9, 42, 42.1, 42.2, 42.3, 42.4, 42.5, 42.6, 42.7, 42.8, 42.9, 43, 43.1, 43.2, 43.3, 43.4, 43.5, 43.6, 43.7, 43.8, 43.9, 44, 44.1, 44.2, 44.3, 44.4, 44.5, 44.6, 44.7, 44.8, 44.9, 45, 45.1, 45.2, 45.3, 45.4, 45.5, 45.6, 45.7, 45.8, 45.9, 46, 46.1, 46.2, 46.3, 46.4, 46.5, 46.6, 46.7, 46.8, 46.9, 47, 47.1, 47.2, 47.3, 47.4, 47.5, 47.6, 47.7, 47.8, 47.9, 48, 48.1, 48.2, 48.3, 48.4, 48.5, 48.6, 48.7, 48.8, 48.9, 49, 49.1, 49.2, 49.3, 49.4, 49.5, 49.6, 49.7, 49.8, 49.9, 50, 50.1, 50.2, 50.3, 50.4, 50.5, 50.6, 50.7, 50.8, 50.9, 51, 51.1, 51.2, 51.3, 51.4, 51.5, 51.6, 51.7, 51.8, 51.9, 52, 52.1, 52.2, 52.3, 52.4, 52.5, 52.6, 52.7, 52.8, 52.9, 53, 53.1, 53.2, 53.3, 53.4, 53.5, 53.6, 53.7, 53.8, 53.9, 54, 54.1, 54.2, 54.3, 54.4, 54.5, 54.6, 54.7, 54.8, 54.9, 55, 55.1, 55.2, 55.3, 55.4, 55.5, 55.6, 55.7, 55.8, 55.9, 56, 56.1, 56.2, 56.3, 56.4, 56.5, 56.6, 56.7, 56.8, 56.9, 57, 57.1, 57.2, 57.3, 57.4, 57.5, 57.6, 57.7, 57.8, 57.9, 58, 58.1, 58.2, 58.3, 58.4, 58.5, 58.6, 58.7, 58.8, 58.9, 59, 59.1, 59.2, 59.3, 59.4, 59.5, 59.6, 59.7, 59.8, 59.9, 60, 60.1, 60.2, 60.3, 60.4, 60.5, 60.6, 60.7, 60.8, 60.9, 61, 61.1, 61.2, 61.3, 61.4, 61.5, 61.6, 61.7, 61.8, 61.9, 62, 62.1, 62.2, 62.3, 62.4, 62.5, 62.6, 62.7, 62.8, 62.9, 63, 63.1, 63.2, 63.3, 63.4, 63.5, 63.6, 63.7, 63.8, 63.9, 64, 64.1, 64.2, 64.3, 64.4, 64.5, 64.6, 64.7, 64.8, 64.9, 65, 65.1, 65.2, 65.3, 65.4, 65.5, 65.6, 65.7, 65.8, 65.9, 66, 66.1, 66.2, 66.3, 66.4, 66.5, 66.6, 66.7, 66.8, 66.9, 67, 67.1, 67.2, 67.3, 67.4, 67.5, 67.6, 67.7, 67.8, 67.9, 68, 68.1, 68.2, 68.3, 68.4, 68.5, 68.6, 68.7, 68.8, 68.9, 69, 69.1, 69.2, 69.3, 69.4, 69.5, 69.6, 69.7, 69.8, 69.9, 70, 70.1, 70.2, 70.3, 70.4, 70.5, 70.6, 70.7, 70.8, 70.9, 71, 71.1, 71.2, 71.3, 71.4, 71.5, 71.6, 71.7, 71.8, 71.9, 72, 72.1, 72.2, 72.3, 72.4, 72.5, 72.6, 72.7, 72.8, 72.9, 73, 73.1, 73.2, 73.3, 73.4, 73.5, 73.6, 73.7, 73.8, 73.9, 74, 74.1, 74.2, 74.3, 74.4, 74.5, 74.6, 74.7, 74.8, 74.9, 75, 75.1, 75.2, 75.3, 75.4, 75.5, 75.6, 75.7, 75.8, 75.9, 76, 76.1, 76.2, 76.3, 76.4, 76.5, 76.6, 76.7, 76.8, 76.9, 77, 77.1, 77.2, 77.3, 77.4, 77.5, 77.6, 77.7, 77.8, 77.9, 78, 78.1, 78.2, 78.3, 78.4, 78.5, 78.6, 78.7, 78.8, 78.9, 79, 79.1, 79.2, 79.3, 79.4, 79.5, 79.6, 79.7, 79.8, 79.9, 80, 80.1, 80.2, 80.3, 80.4, 80.5, 80.6, 80.7, 80.8, 80.9, 81, 81.1, 81.2, 81.3, 81.4, 81.5, 81.6, 81.7, 81.8, 81.9, 82, 82.1, 82.2, 82.3, 82.4, 82.5, 82.6, 82.7, 82.8, 82.9, 83, 83.1, 83.2, 83.3, 83.4, 83.5, 83.6, 83.7, 83.8, 83.9, 84, 84.1, 84.2, 84.3, 84.4, 84.5, 84.6, 84.7, 84.8, 84.9, 85, 85.1, 85.2, 85.3, 85.4, 85.5, 85.6, 85.7, 85.8, 85.9, 86, 86.1, 86.2, 86.3, 86.4, 86.5, 86.6, 86.7, 86.8, 86.9, 87, 87.1, 87.2, 87.3, 87.4, 87.5, 87.6, 87.7, 87.8, 87.9, 88, 88.1, 88.2, 88.3, 88.4, 88.5, 88.6, 88.7, 88.8, 88.9, 89, 89.1, 89.2, 89.3, 89.4, 89.5, 89.6, 89.7, 89.8, 89.9, 90, 90.1, 90.2, 90.3, 90.4, 90.5, 90.6, 90.7, 90.8, 90.9, 91, 91.1, 91.2, 91.3, 91.4, 91.5, 91.6, 91.7, 91.8, 91.9, 92, 92.1, 92.2, 92.3, 92.4, 92.5, 92.6, 92.7, 92.8, 92.9, 93, 93.1, 93.2, 93.3, 93.4, 93.5, 93.6, 93.7, 93.8, 93.9, 94, 94.1, 94.2, 94.3, 94.4, 94.5, 94.6, 94.7, 94.8, 94.9, 95, 95.1, 95.2, 95.3, 95.4, 95.5, 95.6, 95.7, 95.8, 95.9, 96, 96.1, 96.2, 96.3, 96.4, 96.5, 96.6, 96.7, 96.8, 96.9, 97, 97.1, 97.2, 97.3, 97.4, 97.5, 97.6, 97.7, 97.8, 97.9, 98, 98.1, 98.2, 98.3, 98.4, 98.5, 98.6, 98.7, 98.8, 98.9, 99, 99.1, 99.2, 99.3, 99.4, 99.5, 99.6, 99.7, 99.8, 99.9, 100, 100.1, 100.2, 100.3, 100.4, 100.5, 100.6, 100.7, 100.8, 100.9, 101, 101.1, 101.2, 101.3, 101.4, 101.5, 101.6, 101.7, 101.8, 101.9, 102, 102.1, 102.2, 102.3, 102.4, 102.5, 102.6, 102.7, 102.8, 102.9, 103, 103.1, 103.2, 103.3, 103.4, 103.5, 103.6, 103.7, 103.8, 103.9, 104, 104.1, 104.2, 104.3, 104.4, 104.5, 104.6, 104.7, 104.8, 104.9, 105, 105.1, 105.2, 105.3, 105.4, 105.5, 105.6, 105.7, 105.8, 105.9, 106, 106.1, 106.2, 106.3, 106.4, 106.5, 106.6, 106.7, 106.8, 106.9, 107, 107.1, 107.2, 107.3, 107.4, 107.5, 107.6, 107.7, 107.8, 107.9, 108, 108.1, 108.2, 108.3, 108.4, 108.5, 108.6, 108.7, 108.8, 108.9, 109, 109.1, 109.2, 109.3, 109.4, 109.5, 109.6, 109.7, 109.8, 109.9, 110, 110.1, 110.2, 110.3, 110.4, 110.5, 110.6, 110.7, 110.8, 110.9, 111, 111.1, 111.2, 111.3, 111.4, 111.5, 111.6, 111.7, 111.8, 111.9, 112, 112.1, 112.2, 112.3, 112.4, 112.5, 112.6, 112.7, 112.8, 112.9, 113, 113.1, 113.2, 113.3, 113.4, 113.5, 113.6, 113.7, 113.8, 113.9, 114, 114.1, 114.2, 114.3, 114.4, 114.5, 114.6, 114.7, 114.8, 114.9, 115, 115.1, 115.2, 115.3, 115.4, 115.5, 115.6, 115.7, 115.8, 115.9, 116, 116.1, 116.2, 116.3, 116.4, 116.5, 116.6, 116.7, 116.8, 116.9, 117, 117.1, 117.2, 117.3, 117.4, 117.5, 117.6, 117.7, 117.8, 117.9, 118, 118.1, 118.2, 118.3, 118.4, 118.5, 118.6, 118.7, 118.8, 118.9, 119, 119.1, 119.2, 119.3, 119.4, 119.5, 119.6, 119.7, 119.8, 119.9, 120, 120.1, 120.2, 120.3, 120.4, 120.5, 120.6, 120.7, 120.8, 120.9, 121, 121.1, 121.2, 121.3, 121.4, 121.5, 121.6, 121.7, 121.8, 121.9, 122, 122.1, 122.2, 122.3, 122.4, 122.5, 122.6, 122.7, 122.8, 122.9, 123, 123.1, 123.2, 123.3, 123.4, 123.5, 123.6, 123.7, 123.8, 123.9, 124, 124.1, 124.2, 124.3, 124.4, 124.5, 124.6, 124.7, 124.8, 124.9, 125, 125.1, 125.2, 125.3, 125.4, 125.5, 125.6, 125.7, 125.8, 125.9, 126, 126.1, 126.2, 126.3, 126.4, 126.5, 126.6, 126.7, 126.8, 126.9, 127, 127.1, 127.2, 127.3, 127.4, 127.5, 127.6, 127.7, 127.8, 127.9, 128, 128.1, 128.2, 128.3, 128.4, 128.5, 128.6, 128.7, 128.8, 128.9, 129, 129.1, 129.2, 129.3, 129.4, 129.5, 129.6, 129.7, 129.8, 129.9, 130, 130.1, 130.2, 130.3, 130.4, 130.5, 130.6, 130.7, 130.8, 130.9, 131, 131.1, 131.2, 131.3, 131.4, 131.5, 131.6, 131.7, 131.8, 131.9, 132, 132.1, 132.2, 132.3, 132.4, 132.5, 132.6, 132.7, 132.8, 132.9, 133, 133.1, 133.2, 133.3, 133.4, 133.5, 133.6, 133.7, 133.8, 133.9, 134, 134.1, 134.2, 134.3, 134.4, 134.5, 134.6, 134.7, 134.8, 134.9, 135, 135.1, 135.2, 135.3, 135.4, 135.5, 135.6, 135.7, 135.8, 135.9, 136, 136.1, 136.2, 136.3, 136.4, 136.5, 136.6, 136.7, 136.8, 136.9, 137, 137.1, 137.2, 137.3, 137.4, 137.5, 137.6, 137.7, 137.8, 137.9, 138, 138.1, 138.2, 138.3, 138.4, 138.5, 138.6, 138.7, 138.8, 138.9, 139, 139.1, 139.2, 139.3, 139.4, 139.5, 139.6, 139.7, 139.8, 139.9, 140, 140.1, 140.2, 140.3, 140.4, 140.5, 140.6, 140.7, 140.8, 140.9, 141, 141.1, 141.2, 141.3, 141.4, 141.5, 141.6, 141.7, 141.8, 141.9, 142, 142.1, 142.2, 142.3, 142.4, 142.5, 142.6, 142.7, 142.8, 142.9, 143, 143.1, 143.2, 143.3, 143.4, 143.5, 143.6, 143.7, 143.8, 143.9, 144, 144.1, 144.2, 144.3, 144.4, 144.5, 144.6, 144.7, 144.8, 144.9, 145, 145.1, 145.2, 145.3, 145.4, 145.5, 145.6, 145.7, 145.8, 145.9, 146, 146.1, 146.2, 146.3, 146.4, 146.5, 146.6, 146.7, 146.8, 146.9, 147, 147.1, 147.2, 147.3, 147.4, 147.5, 147.6, 147.7, 147.8, 147.9, 148, 148.1, 148.2, 148.3, 148.4, 148.5, 148.6, 148.7, 148.8, 148.9, 149, 149.1, 149.2, 149.3, 149.4, 149.5, 149.6, 149.7, 149.8, 149.9, 150, 150.1, 150.2, 150.3, 150.4, 150.5, 150.6, 150.7, 150.8, 150.9, 151, 151.1, 151.2, 151.3, 151.4, 151.5, 151.6, 151.7, 151.8, 151.9, 152, 152.1, 152.2, 152.3, 152.4, 152.5, 152.6, 152.7, 152.8, 152.9, 153, 153.1, 153.2, 153.3, 153.4, 153.5, 153.6, 153.7, 153.8, 153.9, 154, 154.1, 154.2, 154.3, 154.4, 154.5, 154.6, 154.7, 154.8, 154.9, 155, 155.1, 155.2, 155.3, 155.4, 155.5, 155.6, 155.7, 155.8, 155.9, 156, 156.1, 156.2, 156.3, 156.4, 156.5, 156.6, 156.7, 156.8, 156.9, 157, 157.1, 157.2, 157.3, 157.4, 157.5, 157.6, 157.7, 157.8, 157.9, 158, 158.1, 158.2, 158.3, 158.4, 158.5, 158.6, 158.7, 158.8, 158.9, 159, 159.1, 159.2, 159.3, 159.4, 159.5, 159.6, 159.7, 159.8, 159.9, 160, 160.1, 160.2, 160.3, 160.4, 160.5, 160.6, 160.7, 160.8, 160.9, 161, 161.1, 161.2, 161.3, 161.4, 161.5, 161.6, 161.7, 161.8, 161.9, 162, 162.1, 162.2, 162.3, 162.4, 162.5, 162.6, 162.7, 162.8, 162.9, 163, 163.1, 163.2, 163.3, 163.4, 163.5, 163.6, 163.7, 163.8, 163.9, 164, 164.1, 164.2, 164.3, 164.4, 164.5, 164.6, 164.7, 164.8, 164.9, 165, 165.1, 165.2, 165.3, 165.4, 165.5, 165.6, 165.7, 165.8, 165.9, 166, 166.1, 166.2, 166.3, 166.4, 166.5, 166.6, 166.7, 166.8, 166.9, 167, 167.1, 167.2, 167.3, 167.4, 167.5, 167.6, 167.7, 167.8, 167.9, 168, 168.1, 168.2, 168.3, 168.4, 168.5, 168.6, 168.7, 168.8, 168.9, 169, 169.1, 169.2, 169.3, 169.4, 169.5, 169.6, 169.7, 169.8, 169.9, 170, 170.1, 170.2, 170.3, 170.4, 170.5, 170.6, 170.7, 170.8, 170.9, 171, 171.1, 171.2, 171.3, 171.4, 171.5, 171.6, 171.7, 171.8, 171.9, 172, 172.1, 172.2, 172.3, 172.4, 172.5, 172.6, 172.7, 172.8, 172.9, 173, 173.1, 173.2, 173.3, 173.4, 173.5, 173.6, 173.7, 173.8, 173.9, 174, 174.1, 174.2, 174.3, 174.4, 174.5, 174.6, 174.7, 174.8, 174.9, 175, 175.1, 175.2, 175.3, 175.4, 175.5, 175.6, 175.7, 175.8, 175.9, 176, 176.1, 176.2, 176.3, 176.4, 176.5, 176.6, 176.7, 176.8, 176.9, 177, 177.1, 177.2, 177.3, 177.4, 177.5, 177.6, 177.7, 177.8, 177.9, 178, 178.1, 178.2, 178.3, 178.4, 178.5, 178.6, 178.7, 178.8, 178.9, 179, 179.1, 179.2, 179.3, 179.4, 179.5, 179.6, 179.7, 179.8, 179.9, 180, 180.1, 180.2, 180.3, 180.4, 180.5, 180.6, 180.7, 180.8, 180.9, 181, 181.1, 181.2, 181.3, 181.4, 181.5, 181.6, 181.7, 181.8, 181.9, 182, 182.1, 182.2, 182.3, 182.4, 182.5, 182.6, 182.7, 182.8, 182.9, 183, 183.1, 183.2, 183.3, 183.4, 183.5, 183.6, 183.7, 183.8, 183.9, 184, 184.1, 184.2, 184.3, 184.4, 184.5, 184.6, 184.7, 184.8, 184.9, 185, 185.1, 185.2, 185.3, 185.4, 185.5, 185.6, 185.7, 185.8, 185.9, 186, 186.1, 186.2, 186.3, 186.4, 186.5, 186.6, 186.7, 186.8, 186.9, 187, 187.1, 187.2, 187.3, 187.4, 187.5, 187.6, 187.7, 187.8, 187.9, 188, 188.1, 188.2, 188.3, 188.4, 188.5, 188.6, 188.7, 188.8, 188.9, 189, 189.1, 189.2, 189.3, 189.4, 189.5, 189.6, 189.7, 189.8, 189.9, 190, 190.1, 190.2, 190.3, 190.4, 190.5, 190.6, 190.7, 190.8, 190.9, 191, 191.1, 191.2, 191.3, 191.4, 191.5, 191.6, 191.7, 191.8, 191.9, 192, 192.1, 192.2, 192.3, 192.4, 192.5, 192.6, 192.7, 192.8, 192.9, 193, 193.1, 193.2, 193.3, 193.4, 193.5, 193.6, 193.7, 193.8, 193.9, 194, 194.1, 194.2, 194.3, 194.4, 194.5, 194.6, 194.7, 194.8, 194.9, 195, 195.1, 195.2, 195.3, 195.4, 195.5, 195.6, 195.7, 195.8, 195.9, 196, 196.1, 196.2, 196.3, 196.4, 196.5, 196.6, 196.7, 196.8, 196.9, 197, 197.1, 197.2, 197.3, 197.4, 197.5, 197.6, 197.7, 197.8, 197.9, 198, 198.1, 198.2, 198.3, 198.4, 198.5, 198.6, 198.7, 198.8, 198.9, 199, 199.1, 199.2, 199.3, 199.4, 199.5, 199.6, 199.7, 199.8, 199.9, 200, 200.1, 200.2, 200.3, 200.4, 200.5, 200.6, 200.7, 200.8, 200.9, 201, 201.1, 201.2, 201.3, 201.4, 201.5, 201.6, 201.7, 201.8, 201.9, 202, 202.1, 202.2, 202.3, 202.4, 202.5, 202.6, 202.7, 202.8, 202.9, 203, 203.1, 203.2, 203.3, 203.4, 203.5, 203.6, 203.7, 203.8, 203.9, 204, 204.1, 204.2, 204.3, 204.4, 204.5, 204.6, 204.7, 204.8, 204.9, 205, 205.1, 205.2, 205.3, 205.4, 205.5, 205.6, 205.7, 205.8, 205.9, 206, 206.1, 206.2, 206.3, 206.4, 206.5, 206.6, 206.7, 206.8, 206.9, 207, 207.1, 207.2, 207.3, 207.4, 207.5, 207.6, 207.7, 207.8, 207.9, 208, 208.1, 208.2, 208.3, 208.4, 208.5, 208.6, 208.7, 208.8, 208.9, 209, 209.1, 209.2, 209.3, 209.4, 209.5, 209.6, 209.7, 209.8, 209.9, 210, 210.1, 210.2, 210.3, 210.4, 210.5, 210.6, 210.7, 210.8, 210.9, 211, 211.1, 211.2, 211.3, 211.4, 211.5, 211.6, 211.7, 211.8, 211.9, 212, 212.1, 212.2, 212.3, 212.4, 212.5, 212.6, 212.7, 212.8, 212.9, 213, 213.1, 213.2, 213.3, 213.4, 213.5, 213.6, 213.7, 213.8, 213.9, 214, 214.1, 214.2, 214.3, 214.4, 214.5, 214.6, 214.7, 214.8, 214.9, 215, 215.1, 215.2, 215.3, 215.4, 215.5, 215.6, 215.7, 215.8, 215.9, 216, 216.1, 216.2, 216.3, 216.4, 216.5, 216.6, 216.7, 216.8, 216.9, 217, 217.1, 217.2, 217.3, 217.4, 217.5, 217.6, 217.7, 217.8, 217.9, 218, 218.1, 218.2, 218.3, 218.4, 218.5, 218.6, 218.7, 218.8, 218.9, 219, 219.1, 219.2, 219.3, 219.4, 219.5, 219.6, 219.7, 219.8, 219.9, 220, 220.1, 220.2, 220.3, 220.4, 220.5, 220.6, 220.7, 220.8, 220.9, 221, 221.1, 221.2, 221.3, 221.4, 221.5, 221.6, 221.7, 221.8, 221.9, 222, 222.1, 222.2, 222.3, 222.4, 222.5, 222.6, 222.7, 222.8, 222.9, 223, 223.1, 223.2, 223.3, 223.4, 223.5, 223.6, 223.7, 223.8, 223.9, 224, 224.1, 224.2, 224.3, 224.4, 224.5, 224.6, 224.7, 224.8, 224.9, 225, 225.1, 225.2, 225.3, 225.4, 225.5, 225.6, 225.7, 225.8, 225.9, 226, 226.1, 226.2, 226.3, 226.4, 226.5, 226.6, 226.7, 226.8, 226.9, 227, 227.1, 227.2, 227.3, 227.4, 227.5, 227.6, 227.7, 227.8, 227.9, 228, 228.1, 228.2, 228.3, 228.4, 228.5, 228.6, 228.7, 228.8, 228.9, 229, 229.1, 229.2, 229.3, 229.4, 229.5, 229.6, 229.7, 229.8, 229.9, 230, 230.1, 230.2, 230.3, 230.4, 230.5, 230.6, 230.7, 230.8, 230.9, 231, 231.1, 231.2, 231.3, 231.4, 231.5, 231.6, 231.7, 231.8, 231.9, 232, 232.1, 232.2, 232.3, 232.4, 232.5, 232.6, 232.7, 232.8, 232.9, 233, 233.1, 233.2, 233.3, 233.4, 233.5, 233.6, 233.7, 233.8, 233.9, 234, 234.1, 234.2, 234.3, 234.4, 234.5, 234.6, 234.7, 234.8, 234.9, 235, 235.1, 235.2, 235.3, 235.4, 235.5, 235.6, 235.7, 235.8, 235.9, 236, 236.1, 236.2, 236.3, 236.4, 236.5, 236.6, 236.7, 236.8, 236.9, 237, 237.1, 237.2, 237.3, 237.4, 237.5, 237.6, 237.7, 237.8, 237.9, 238, 238.1, 238.2, 238.3, 238.4, 238.5, 238.6, 238.7, 238.8, 238.9, 239, 239.1, 239.2, 239.3, 239.4, 239.5, 239.6, 239.7, 239.8, 239.9, 240, 240.1, 240.2, 240.3, 240.4, 240.5, 240.6, 240.7, 240.8, 240.9, 241, 241.1, 241.2, 241.3, 241.4, 241.5, 241.6, 241.7, 241.8, 241.9, 242, 242.1, 242.2, 242.3, 242.4, 242.5, 242.6, 242.7, 242.8, 242.9, 243, 243.1, 243.2, 243.3, 243.4, 243.5, 243.6, 243.7, 243.8, 243.9, 244, 244.1, 244.2, 244.3, 244.4, 244.5, 244.6, 244.7, 244.8, 244.9, 245, 245.1, 245.2, 245.3, 245.4, 245.5, 245.6, 245.7, 245.8, 245.9, 246, 246.1, 246.2, 246.3, 246.4, 246.5, 246.6, 246.7, 246.8, 246.9, 247, 247.1, 247.2, 247.3, 247.4, 247.5, 247.6, 247.7, 247.8, 247.9, 248, 248.1, 248.2, 248.3, 248.4, 248.5, 248.6, 248.7, 248.8, 248.9, 249, 249.1, 249.2, 249.3, 249.4, 249.5, 249.6, 249.7, 249.8, 249.9, 250, 250.1, 250.2, 250.3, 250.4, 250.5, 250.6, 250.7, 250.8, 250.9, 251, 251.1, 251.2, 251.3, 251.4, 251.5, 251.6, 251.7, 251.8, 251.9, 252, 252.1, 252.2, 252.3, 252.4, 252.5, 252.6, 252.7, 252.8, 252.9, 253, 253.1, 253.2, 253.3, 253.4, 253.5, 253.6, 253.7, 253.8, 253.9, 254, 254.1, 254.2, 254.3, 254.4, 254.5, 254.6, 254.7, 254.8, 254.9, 255, 255.1, 255.2, 255.3, 255.4, 255.5, 255.6, 255.7, 255.8, 255.9, 256, 256.1, 256.2, 256.3, 256.4, 256.5, 256.6, 256.7, 256.8, 256.9, 257, 257.1, 257.2, 257.3, 257.4, 257.5, 257.6, 257.7, 257.8, 257.9, 258, 258.1, 258.2, 258.3, 258.4, 258.5, 258.6, 258.7, 258.8, 258.9, 259, 259.1, 259.2, 259.3, 259.4, 259.5, 259.6, 259.7, 259.8, 259.9, 260, 260.1, 260.2, 260.3, 260.4, 260.5, 260.6, 260.7, 260.8, 260.9, 261, 261.1, 261.2, 261.3, 261.4, 261.5, 261.6, 261.7, 261.8, 261.9, 262, 262.1, 262.2, 262.3, 262.4, 262.5, 262.6, 262.7, 262.8, 262.9, 263, 263.1, 263.2, 263.3, 263.4, 263.5, 263.6, 263.7, 263.8, 263.9, 264, 264.1, 264.2, 264.3, 264.4, 264.5, 264.6, 264.7, 264.8, 264.9, 265, 265.1, 265.2, 265.3, 265.4, 265.5, 265.6, 265.7, 265.8, 265.9, 266, 266.1, 266.2, 266.3, 266.4, 266.5, 266.6, 266.7, 266.8, 266.9, 267, 267.1, 267.2, 267.3, 267.4, 267.5, 267.6, 267.7, 267.8, 267.9, 268, 268.1, 268.2, 268.3, 268.4, 268.5, 268.6, 268.7, 268.8, 268.9, 269, 269.1, 269.2, 269.3, 269.4, 269.5, 269.6, 269.7, 269.8, 269.9, 270, 270.1, 270.2, 270.3, 270.4, 270.5, 270.6, 270.7, 270.8, 270.9, 271, 271.1, 271.2, 271.3, 271.4, 271.5, 271.6, 271.7, 271.8, 271.9, 272, 272.1, 272.2, 272.3, 272.4, 272.5, 272.6, 272.7, 272.8, 272.9, 273, 273.1, 273.2, 273.3, 273.4, 273.5, 273.6, 273.7, 273.8, 273.9, 274, 274.1, 274.2, 274.3, 274.4, 274.5, 274.6, 274.7, 274.8, 274.9, 275, 275.1, 275.2, 275.3, 275.4, 275.5, 275.6, 275.7, 275.8, 275.9, 276, 276.1, 276.2, 276.3, 276.4, 276.5, 276.6, 276.7, 276.8, 276.9, 277, 277.1, 277.2, 277.3, 277.4, 277.5, 277.6, 277.7, 277.8, 277.9, 278, 278.1, 278.2, 278.3, 278.4, 278.5, 278.6, 278.7, 278.8, 278.9, 279, 279.1, 279.2, 279.3, 279.4, 279.5, 279.6, 279.7, 279.8, 279.9, 280, 280.1, 280.2, 280.3, 280.4, 280.5, 280.6, 280.7, 280.8, 280.9, 281, 281.1, 281.2, 281.3, 281.4, 281.5, 281.6, 281.7, 281.8, 281.9, 282, 282.1, 282.2, 282.3, 282.4, 282.5, 282.6, 282.7, 282.8, 282.9, 283, 283.1, 283.2, 283.3, 283.4, 283.5, 283.6, 283.7, 283.8, 283.9, 284, 284.1, 284.2, 284.3, 284.4, 284.5, 284.6, 284.7, 284.8, 284.9, 285, 285.1, 285.2, 285.3, 285.4, 285.5, 285.6, 285.7, 285.8, 285.9, 286, 286.1, 286.2, 286.3, 286.4, 286.5, 286.6, 286.7, 286.8, 286.9, 287, 287.1, 287.2, 287.3, 287.4, 287.5, 287.6, 287.7, 287.8, 287.9, 288, 288.1, 288.2, 288.3, 288.4, 288.5, 288.6, 288.7, 288.8, 288.9, 289, 289.1, 289.2, 289.3, 289.4, 289.5, 289.6, 289.7, 289.8, 289.9, 290, 290.1, 290.2, 290.3, 290.4, 290.5, 290.6, 290.7, 290.8, 290.9, 291, 291.1, 291.2, 291.3, 291.4, 291.5, 291.6, 291.7, 291.8, 291.9, 292, 292.1, 292.2, 292.3, 292.4, 292.5, 292.6, 292.7, 292.8, 292.9, 293, 293.1, 293.2, 293.3, 293.4, 293.5, 293.6, 293.7, 293.8, 293.9, 294, 294.1, 294.2, 294.3, 294.4, 294.5, 294.6, 294.7, 294.8, 294.9, 295, 295.1, 295.2, 295.3, 295.4, 295.5, 295.6, 295.7, 295.8, 295.9, 296, 296.1, 296.2, 296.3, 296.4, 296.5, 296.6, 296.7, 296.8, 296.9, 297, 297.1, 297.2, 297.3, 297.4, 297.5, 297.6, 297.7, 297.8, 297.9, 298, 298.1, 298.2, 298.3, 298.4, 298.5, 298.6, 298.7, 298.8, 298.9, 299, 299.1, 299.2, 299.3, 299.4, 299.5, 299.6, 299.7, 299.8, 299.9, 300, 300.1, 300.2, 300.3, 300.4, 300.5, 300.6, 300.7, 300.8, 300.9, 301, 301.1, 301.2, 301.3, 301.4, 301.5, 301.6, 301.7, 301.8, 301.9, 302, 302.1, 302.2, 302.3, 302.4, 302.5, 302.6, 302.7, 302.8, 302.9, 303, 303.1, 303.2, 303.3, 303.4, 303.5, 303.6, 303.7, 303.8, 303.9, 304, 304.1, 304.2, 304.3, 304.4, 304.5, 304.6, 304.7, 304.8, 304.9, 305, 305.1, 305.2, 305.3, 305.4, 305.5, 305.6, 305.7, 305.8, 305.9, 306, 306.1, 306.2, 306.3, 306.4, 306.5, 306.6, 306.7, 306.8, 306.9, 307, 307.1, 307.2, 307.3, 307.4, 307.5, 307.6, 307.7, 307.8, 307.9, 308, 308.1, 308.2, 308.3, 308.4, 308.5, 308.6, 308.7, 308.8, 308.9, 309, 309.1, 309.2, 309.3, 309.4, 309.5, 309.6, 309.7, 309.8, 309.9, 310, 310.1, 310.2, 310.3, 310.4, 310.5, 310.6, 310.7, 310.8, 310.9, 311, 311.1, 311.2, 311.3, 311.4, 311.5, 311.6, 311.7, 311.8, 311.9, 312, 312.1, 312.2, 312.3, 312.4, 312.5, 312.6, 312.7, 312.8, 312.9, 313, 313.1, 313.2, 313.3, 313.4, 313.5, 313.6, 313.7, 313.8, 313.9, 314, 314.1, 314.2, 314.3, 314.4, 314.5, 314.6, 314.7, 314.8, 314.9, 315, 315.1, 315.2, 315.3, 315.4, 315.5, 315.6, 315.7, 315.8, 315.9, 316, 316.1, 316.2, 316.3, 316.4, 316.5, 316.6, 316.7, 316.8, 316.9, 317, 317.1, 317.2, 317.3, 317.4, 317.5, 317.6, 317.7, 317.8, 317.9, 318, 318.1, 318.2, 318.3, 318.4, 318.5, 318.6, 318.7, 318.8, 318.9, 319, 319.1, 319.2, 319.3, 319.4, 319.5, 319.6, 319.7, 319.8, 319.9, 320, 320.1, 320.2, 320.3, 320.4, 320.5, 320.6, 320.7, 320.8, 320.9, 321, 321.1, 321.2, 321.3, 321.4, 321.5, 321.6, 321.7, 321.8, 321.9, 322, 322.1, 322.2, 322.3, 322.4, 322.5, 322.6, 322.7, 322.8, 322.9, 323, 323.1, 323.2, 323.3, 323.4, 323.5, 323.6, 323.7, 323.8, 323.9, 324, 324.1, 324.2, 324.3, 324.4, 324.5, 324.6, 324.7, 324.8, 324.9, 325, 325.1, 325.2, 325.3, 325.4, 325.5, 325.6, 325.7, 325.8, 325.9, 326, 326.1, 326.2, 326.3, 326.4, 326.5, 326.6, 326.7, 326.8, 326.9, 327, 327.1, 327.2, 327.3, 327.4, 327.5, 327.6, 327.7, 327.8, 327.9, 328, 328.1, 328.2, 328.3, 328.4, 328.5, 328.6, 328.7, 328.8, 328.9, 329, 329.1, 329.2, 329.3, 329.4, 329.5, 329.6, 329.7, 329.8, 329.9, 330, 330.1, 330.2, 330.3, 330.4, 330.5, 330.6, 330.7, 330.8, 330.9, 331, 331.1, 331.2, 331.3, 331.4, 331.5, 331.6, 331.7, 331.8, 331.9, 332, 332.1, 332.2, 332.3, 332.4, 332.5, 332.6, 332.7, 332.8, 332.9, 333, 333.1, 333.2, 333.3, 333.4, 333.5, 333.6, 333.7, 333.8, 333.9, 334, 334.1, 334.2, 334.3, 334.4, 334.5, 334.6, 334.7, 334.8, 334.9, 335, 335.1, 335.2, 335.3, 335.4, 335.5, 335.6, 335.7, 335.8, 335.9, 336, 336.1, 336.2, 336.3, 336.4, 336.5, 336.6, 336.7, 336.8, 336.9, 337, 337.1, 337.2, 337.3, 337.4, 337.5, 337.6, 337.7, 337.8, 337.9, 338, 338.1, 338.2, 338.3, 338.4, 338.5, 338.6, 338.7, 338.8, 338.9, 339, 339.1, 339.2, 339.3, 339.4, 339.5, 339.6, 339.7, 339.8, 339.9, 340, 340.1, 340.2, 340.3, 340.4, 340.5, 340.6, 340.7, 340.8, 340.9, 341, 341.1, 341.2, 341.3, 341.4, 341.5, 341.6, 341.7, 341.8, 341.9, 342, 342.1, 342.2, 342.3, 342.4, 342.5, 342.6, 342.7, 342.8, 342.9, 343, 343.1, 343.2, 343.3, 343.4, 343.5, 343.6, 343.7, 343.8, 343.9, 344, 344.1, 344.2, 344.3, 344.4, 344.5, 344.6, 344.7, 344.8, 344.9, 345, 345.1, 345.2, 345.3, 345.4, 345.5, 345.6, 345.7, 345.8, 345.9, 346, 346.1, 346.2, 346.3, 346.4, 346.5, 346.6, 346.7, 346.8, 346.9, 347, 347.1, 347.2, 347.3, 347.4, 347.5, 347.6, 347.7, 347.8, 347.9, 348, 348.1, 348.2, 348.3, 348.4, 348.5, 348.6, 348.7, 348.8, 348.9, 349, 349.1, 349.2, 349.3, 349.4, 349.5, 349.6, 349.7, 349.8, 349.9, 350, 350.1, 350.2, 350.3, 350.4, 350.5, 350.6, 350.7, 350.8, 350.9, 351, 351.1, 351.2, 351.3, 351.4, 351.5, 351.6, 351.7, 351.8, 351.9, 352, 352.1, 352.2, 352.3, 352.4, 352.5, 352.6, 352.7, 352.8, 352.9, 353, 353.1, 353.2, 353.3, 353.4, 353.5, 353.6, 353.7, 353.8, 353.9, 354, 354.1, 354.2, 354.3, 354.4, 354.5, 354.6, 354.7, 354.8, 354.9, 355, 355.1, 355.2, 355.3, 355.4, 355.5, 355.6, 355.7, 355.8, 355.9, 356, 356.1, 356.2, 356.3, 356.4, 356.5, 356.6, 356.7, 356.8, 356.9, 357, 357.1, 357.2, 357.3, 357.4, 357.5, 357.6, 357.7, 357.8, 357.9, 358, 358.1, 358.2, 358.3, 358.4, 358.5, 358.6, 358.7, 358.8, 358.9, 359, 359.1, 359.2, 359.3, 359.4, 359.5, 359.6, 359.7, 359.8, 359.9, 360, 360.1, 360.2, 360.3, 360.4, 360.5, 360.6, 360.7, 360.8, 360.9, 361, 361.1, 361.2, 361.3, 361.4, 361.5, 361.6, 361.7, 361.8, 361.9, 362, 362.1, 362.2, 362.3, 362.4, 362.5, 362.6, 362.7, 362.8, 362.9, 363, 363.1, 363.2, 363.3, 363.4, 363.5, 363.6, 363.7, 363.8, 363.9, 364, 364.1, 364.2, 364.3, 364.4, 364.5, 364.6, 364.7, 364.8, 364.9, 365, 365.1, 365.2, 365.3, 365.4, 365.5, 365.6, 365.7, 365.8, 365.9, 366, 366.1, 366.2, 366.3, 366.4, 366.5, 366.6, 366.7, 366.8, 366.9, 367, 367.1, 367.2, 367.3, 367.4, 367.5, 367.6, 367.7, 367.8, 367.9, 368, 368.1, 368.2, 368.3, 368.4, 368.5, 368.6, 368.7, 368.8, 368.9, 369, 369.1, 369.2, 369.3, 369.4, 369.5, 369.6, 369.7, 369.8, 369.9, 370, 370.1, 370.2, 370.3, 370.4, 370.5, 370.6, 370.7, 370.8, 370.9, 371, 371.1, 371.2, 371.3, 371.4, 371.5, 371.6, 371.7, 371.8, 371.9, 372, 372.1, 372.2, 372.3, 372.4, 372.5, 372.6, 372.7, 372.8, 372.9, 373, 373.1, 373.2, 373.3, 373.4, 373.5, 373.6, 373.7, 373.8, 373.9, 374, 374.1, 374.2, 374.3, 374.4, 374.5, 374.6, 374.7, 374.8, 374.9, 375, 375.1, 375.2, 375.3, 375.4, 375.5, 375.6, 375.7, 375.8, 375.9, 376, 376.1, 376.2, 376.3, 376.4, 376.5, 376.6, 376.7, 376.8, 376.9, 377, 377.1, 377.2, 377.3, 377.4, 377.5, 377.6, 377.7, 377.8, 377.9, 378, 378.1, 378.2, 378.3, 378.4, 378.5, 378.6, 378.7, 378.8, 378.9, 379, 379.1, 379.2, 379.3, 379.4, 379.5, 379.6, 379.7, 379.8, 379.9, 380, 380.1, 380.2, 380.3, 380.4, 380.5, 380.6, 380.7, 380.8, 380.9, 381, 381.1, 381.2, 381.3, 381.4, 381.5, 381.6, 381.7, 381.8, 381.9, 382, 382.1, 382.2, 382.3, 382.4, 382.5, 382.6, 382.7, 382.8, 382.9, 383, 383.1, 383.2, 383.3, 383.4, 383.5, 383.6, 383.7, 383.8, 383.9, 384, 384.1, 384.2, 384.3, 384.4, 384.5, 384.6, 384.7, 384.8, 384.9, 385, 385.1, 385.2, 385.3, 385.4, 385.5, 385.6, 385.7, 385.8, 385.9, 386, 386.1, 386.2, 386.3, 386.4, 386.5, 386.6, 386.7, 386.8, 386.9, 387, 387.1, 387.2, 387.3, 387.4, 387.5, 387.6, 387.7, 387.8, 387.9, 388, 388.1, 388.2, 388.3, 388.4, 388.5, 388.6, 388.7, 388.8, 388.9, 389, 389.1, 389.2, 389.3, 389.4, 389.5, 389.6, 389.7, 389.8, 389.9, 390, 390.1, 390.2, 390.3, 390.4, 390.5, 390.6, 390.7, 390.8, 390.9, 391, 391.1, 391.2, 391.3, 391.4, 391.5, 391.6, 391.7, 391.8, 391.9, 392, 392.1, 392.2, 392.3, 392.4, 392.5, 392.6, 392.7, 392.8, 392.9, 393, 393.1, 393.2, 393.3, 393.4, 393.5, 393.6, 393.7, 393.8, 393.9, 394, 394.1, 394.2, 394.3, 394.4, 394.5, 394.6, 394.7, 394.8, 394.9, 395, 395.1, 395.2, 395.3, 395.4, 395.5, 395.6, 395.7, 395.8, 395.9, 396, 396.1, 396.2, 396.3, 396.4, 396.5, 396.6, 396.7, 396.8, 396.9, 397, 397.1, 397.2, 397.3, 397.4, 397.5, 397.6, 397.7, 397.8, 397.9, 398, 398.1, 398.2, 398.3, 398.4, 398.5, 398.6, 398.7, 398.8, 398.9, 399, 399.1, 399.2, 399.3, 399.4, 399.5, 399.6, 399.7, 399.8, 399.9, 400, 400.1, 400.2, 400.3, 400.4, 400.5, 400.6, 400.7, 400.8, 400.9, 401, 401.1, 401.2, 401.3, 401.4, 401.5, 401.6, 401.7, 401.8, 401.9, 402, 402.1, 402.2, 402.3, 402.4, 402.5, 402.6, 402.7, 402.8, 402.9, 403, 403.1, 403.2, 403.3, 403.4, 403.5, 403.6, 403.7, 403.8, 403.9, 404, 404.1, 404.2, 404.3, 404.4, 404.5, 404.6, 404.7, 404.8, 404.9, 405, 405.1, 405.2, 405.3, 405.4, 405.5, 405.6, 405.7, 405.8, 405.9, 406, 406.1, 406.2, 406.3, 406.4, 406.5, 406.6, 406.7, 406.8, 406.9, 407, 407.1, 407.2, 407.3, 407.4, 407.5, 407.6, 407.7, 407.8, 407.9, 408, 408.1, 408.2, 408.3, 408.4, 408.5, 408.6, 408.7, 408.8, 408.9, 409, 409.1, 409.2, 409.3, 409.4, 409.5, 409.6, 409.7, 409.8, 409.9, 410, 410.1, 410.2, 410.3, 410.4, 410.5, 410.6, 410.7, 410.8, 410.9, 411, 411.1, 411.2, 411.3, 411.4, 411.5, 411.6, 411.7, 411.8, 411.9, 412, 412.1, 412.2, 412.3, 412.4, 412.5, 412.6, 412.7, 412.8, 412.9, 413, 413.1, 413.2, 413.3, 413.4, 413.5, 413.6, 413.7, 413.8, 413.9, 414, 414.1, 414.2, 414.3, 414.4, 414.5, 414.6, 414.7, 414.8, 414.9, 415, 415.1, 415.2, 415.3, 415.4, 415.5, 415.6, 415.7, 415.8, 415.9, 416, 416.1, 416.2, 416.3, 416.4, 416.5, 416.6, 416.7, 416.8, 416.9, 417, 417.1, 417.2, 417.3, 417.4, 417.5, 417.6, 417.7, 417.8, 417.9, 418, 418.1, 418.2, 418.3, 418.4, 418.5, 418.6, 418.7, 418.8, 418.9, 419, 419.1, 419.2, 419.3, 419.4, 419.5, 419.6, 419.7, 419.8, 419.9, 420, 420.1, 420.2, 420.3, 420.4, 420.5, 420.6, 420.7, 420.8, 420.9, 421, 421.1, 421.2, 421.3, 421.4, 421.5, 421.6, 421.7, 421.8, 421.9, 422, 422.1, 422.2, 422.3, 422.4, 422.5, 422.6, 422.7, 422.8, 422.9, 423, 423.1, 423.2, 423.3, 423.4, 423.5, 423.6, 423.7, 423.8, 423.9, 424, 424.1, 424.2, 424.3, 424.4, 424.5, 424.6, 424.7, 424.8, 424.9, 425, 425.1, 425.2, 425.3, 425.4, 425.5, 425.6, 425.7, 425.8, 425.9, 426, 426.1, 426.2, 426.3, 426.4, 426.5, 426.6, 426.7, 426.8, 426.9, 427, 427.1, 427.2, 427.3, 427.4, 427.5, 427.6, 427.7, 427.8, 427.9, 428, 428.1, 428.2, 428.3, 428.4, 428.5, 428.6, 428.7, 428.8, 428.9, 429, 429.1, 429.2, 429.3, 429.4, 429.5, 429.6, 429.7, 429.8, 429.9, 430, 430.1, 430.2, 430.3, 430.4, 430.5, 430.6, 430.7, 430.8, 430.9, 431, 431.1, 431.2, 431.3, 431.4, 431.5, 431.6, 431.7, 431.8, 431.9, 432, 432.1, 432.2, 432.3, 432.4, 432.5, 432.6, 432.7, 432.8, 432.9, 433, 433.1, 433.2, 433.3, 433.4, 433.5, 433.6, 433.7, 433.8, 433.9, 434, 434.1, 434.2, 434.3, 434.4, 434.5, 434.6, 434.7, 434.8, 434.9, 435, 435.1, 435.2, 435.3, 435.4, 435.5, 435.6, 435.7, 435.8, 435.9, 436, 436.1, 436.2, 436.3, 436.4, 436.5, 436.6, 436.7, 436.8, 436.9, 437, 437.1, 437.2, 437.3, 437.4, 437.5, 437.6, 437.7, 437.8, 437.9, 438, 438.1, 438.2, 438.3, 438.4, 438.5, 438.6, 438.7, 438.8, 438.9, 439, 439.1, 439.2, 439.3, 439.4, 439.5, 439.6, 439.7, 439.8, 439.9, 440, 440.1, 440.2, 440.3, 440.4, 440.5, 440.6, 440.7, 440.8, 440.9, 441, 441.1, 441.2, 441.3, 441.4, 441.5, 441.6, 441.7, 441.8, 441.9, 442, 442.1, 442.2, 442.3, 442.4, 442.5, 442.6, 442.7, 442.8, 442.9, 443, 443.1, 443.2, 443.3, 443.4, 443.5, 443.6, 443.7, 443.8, 443.9, 444, 444.1, 444.2, 444.3, 444.4, 444.5, 444.6, 444.7, 444.8, 444.9, 445, 445.1, 445.2, 445.3, 445.4, 445.5, 445.6, 445.7, 445.8, 445.9, 446, 446.1, 446.2, 446.3, 446.4, 446.5, 446.6, 446.7, 446.8, 446.9, 447, 447.1, 447.2, 447.3, 447.4, 447.5, 447.6, 447.7, 447.8, 447.9, 448, 448.1, 448.2, 448.3, 448.4, 448.5, 448.6, 448.7, 448.8, 448.9, 449, 449.1, 449.2, 449.3, 449.4, 449.5, 449.6, 449.7, 449.8, 449.9, 450, 450.1, 450.2, 450.3, 450.4, 450.5, 450.6, 450.7, 450.8, 450.9, 451, 451.1, 451.2, 451.3, 451.4, 451.5, 451.6, 451.7, 451.8, 451.9, 452, 452.1, 452.2, 452.3, 452.4, 452.5, 452.6, 452.7, 452.8, 452.9, 453, 453.1, 453.2, 453.3, 453.4, 453.5, 453.6, 453.7, 453.8, 453.9, 454, 454.1, 454.2, 454.3, 454.4, 454.5, 454.6, 454.7, 454.8, 454.9, 455, 455.1, 455.2, 455.3, 455.4, 455.5, 455.6, 455.7, 455.8, 455.9, 456, 456.1, 456.2, 456.3, 456.4, 456.5, 456.6, 456.7, 456.8, 456.9, 457, 457.1, 457.2, 457.3, 457.4, 457.5, 457.6, 457.7, 457.8, 457.9, 458, 458.1, 458.2, 458.3, 458.4, 458.5, 458.6, 458.7, 458.8, 458.9, 459, 459.1, 459.2, 459.3, 459.4, 459.5, 459.6, 459.7, 459.8, 459.9, 460, 460.1, 460.2, 460.3, 460.4, 460.5, 460.6, 460.7, 460.8, 460.9, 461, 461.1, 461.2, 461.3, 461.4, 461.5, 461.6, 461.7, 461.8, 461.9, 462, 462.1, 462.2, 462.3, 462.4, 462.5, 462.6, 462.7, 462.8, 462.9, 463, 463.1, 463.2, 463.3, 463.4, 463.5, 463.6, 463.7, 463.8, 463.9, 464, 464.1, 464.2, 464.3, 464.4, 464.5, 464.6, 464.7, 464.8, 464.9, 465, 465.1, 465.2, 465.3, 465.4, 465.5, 465.6, 465.7, 465.8, 465.9, 466, 466.1, 466.2, 466.3, 466.4, 466.5, 466.6, 466.7, 466.8, 466.9, 467, 467.1, 467.2, 467.3, 467.4, 467.5, 467.6, 467.7, 467.8, 467.9, 468, 468.1, 468.2, 468.3, 468.4, 468.5, 468.6, 468.7, 468.8, 468.9, 469, 469.1, 469.2, 469.3, 469.4, 469.5, 469.6, 469.7, 469.8, 469.9, 470, 470.1, 470.2, 470.3, 470.4, 470.5, 470.6, 470.7, 470.8, 470.9, 471, 471.1, 471.2, 471.3, 471.4, 471.5, 471.6, 471.7, 471.8, 471.9, 472, 472.1, 472.2, 472.3, 472.4, 472.5, 472.6, 472.7, 472.8, 472.9, 473, 473.1, 473.2, 473.3, 473.4, 473.5, 473.6, 473.7, 473.8, 473.9, 474, 474.1, 474.2, 474.3, 474.4, 474.5, 474.6, 474.7, 474.8, 474.9, 475, 475.1, 475.2, 475.3, 475.4, 475.5, 475.6, 475.7, 475.8, 475.9, 476, 476.1, 476.2, 476.3, 476.4, 476.5, 476.6, 476.7, 476.8, 476.9, 477, 477.1, 477.2, 477.3, 477.4, 477.5, 477.6, 477.7, 477.8, 477.9, 478, 478.1, 478.2, 478.3, 478.4, 478.5, 478.6, 478.7, 478.8, 478.9, 479, 479.1, 479.2, 479.3, 479.4, 479.5, 479.6, 479.7, 479.8, 479.9, 480, 480.1, 480.2, 480.3, 480.4, 480.5, 480.6, 480.7, 480.8, 480.9, 481, 481.1, 481.2, 481.3, 481.4, 481.5, 481.6, 481.7, 481.8, 481.9, 482, 482.1, 482.2, 482.3, 482.4, 482.5, 482.6, 482.7, 482.8, 482.9, 483, 483.1, 483.2, 483.3, 483.4, 483.5, 483.6, 483.7, 483.8, 483.9, 484, 484.1, 484.2, 484.3, 484.4, 484.5, 484.6, 484.7, 484.8, 484.9, 485, 485.1, 485.2, 485.3, 485.4, 485.5, 485.6, 485.7, 485.8, 485.9, 486, 486.1, 486.2, 486.3, 486.4, 486.5, 486.6, 486.7, 486.8, 486.9, 487, 487.1, 487.2, 487.3, 487.4, 487.5, 487.6, 487.7, 487.8, 487.9, 488, 488.1, 488.2, 488.3, 488.4, 488.5, 488.6, 488.7, 488.8, 488.9, 489, 489.1, 489.2, 489.3, 489.4, 489.5, 489.6, 489.7, 489.8, 489.9, 490, 490.1, 490.2, 490.3, 490.4, 490.5, 490.6, 490.7, 490.8, 490.9, 491, 491.1, 491.2, 491.3, 491.4, 491.5, 491.6, 491.7, 491.8, 491.9, 492, 492.1, 492.2, 492.3, 492.4, 492.5, 492.6, 492.7, 492.8, 492.9, 493, 493.1, 493.2, 493.3, 493.4, 493.5, 493.6, 493.7, 493.8, 493.9, 494, 494.1, 494.2, 494.3, 494.4, 494.5, 494.6, 494.7, 494.8, 494.9, 495, 495.1, 495.2, 495.3, 495.4, 495.5, 495.6, 495.7, 495.8, 495.9, 496, 496.1, 496.2, 496.3, 496.4, 496.5, 496.6, 496.7, 496.8, 496.9, 497, 497.1, 497.2, 497.3, 497.4, 497.5, 497.6, 497.7, 497.8, 497.9, 498, 498.1, 498.2, 498.3, 498.4, 498.5, 498.6, 498.7, 498.8, 498.9, 499, 499.1, 499.2, 499.3, 499.4, 499.5, 499.6, 499.7, 499.8, 499.9, or 500 mg/ml.

In some embodiments, each cannabinoid is present in the water soluble powder compositions of the invention in an amount of approximately 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20, 20.1, 20.2, 20.3, 20.4, 20.5, 20.6, 20.7, 20.8, 20.9, 21, 21.1, 21.2, 21.3, 21.4, 21.5, 21.6, 21.7, 21.8, 21.9, 22, 22.1, 22.2, 22.3, 22.4, 22.5, 22.6, 22.7, 22.8, 22.9, 23, 23.1, 23.2, 23.3, 23.4, 23.5, 23.6, 23.7, 23.8, 23.9, 24, 24.1, 24.2, 24.3, 24.4, 24.5, 24.6, 24.7, 24.8, 24.9, 25, 25.1, 25.2, 25.3, 25.4, 25.5, 25.6, 25.7, 25.8, 25.9, 26, 26.1, 26.2, 26.3, 26.4, 26.5, 26.6, 26.7, 26.8, 26.9, 27, 27.1, 27.2, 27.3, 27.4, 27.5, 27.6, 27.7, 27.8, 27.9, 28, 28.1, 28.2, 28.3, 28.4, 28.5, 28.6, 28.7, 28.8, 28.9, 29, 29.1, 29.2, 29.3, 29.4, 29.5, 29.6, 29.7, 29.8, 29.9, 30, 30.1, 30.2, 30.3, 30.4, 30.5, 30.6, 30.7, 30.8, 30.9, 31, 31.1, 31.2, 31.3, 31.4, 31.5, 31.6, 31.7, 31.8, 31.9, 32, 32.1, 32.2, 32.3, 32.4, 32.5, 32.6, 32.7, 32.8, 32.9, 33, 33.1, 33.2, 33.3, 33.4, 33.5, 33.6, 33.7, 33.8, 33.9, 34, 34.1, 34.2, 34.3, 34.4, 34.5, 34.6, 34.7, 34.8, 34.9, 35, 35.1, 35.2, 35.3, 35.4, 35.5, 35.6, 35.7, 35.8, 35.9, 36, 36.1, 36.2, 36.3, 36.4, 36.5, 36.6, 36.7, 36.8, 36.9, 37, 37.1, 37.2, 37.3, 37.4, 37.5, 37.6, 37.7, 37.8, 37.9, 38, 38.1, 38.2, 38.3, 38.4, 38.5, 38.6, 38.7, 38.8, 38.9, 39, 39.1, 39.2, 39.3, 39.4, 39.5, 39.6, 39.7, 39.8, 39.9, 40, 40.1, 40.2, 40.3, 40.4, 40.5, 40.6, 40.7, 40.8, 40.9, 41, 41.1, 41.2, 41.3, 41.4, 41.5, 41.6, 41.7, 41.8, 41.9, 42, 42.1, 42.2, 42.3, 42.4, 42.5, 42.6, 42.7, 42.8, 42.9, 43, 43.1, 43.2, 43.3, 43.4, 43.5, 43.6, 43.7, 43.8, 43.9, 44, 44.1, 44.2, 44.3, 44.4, 44.5, 44.6, 44.7, 44.8, 44.9, 45, 45.1, 45.2, 45.3, 45.4, 45.5, 45.6, 45.7, 45.8, 45.9, 46, 46.1, 46.2, 46.3, 46.4, 46.5, 46.6, 46.7, 46.8, 46.9, 47, 47.1, 47.2, 47.3, 47.4, 47.5, 47.6, 47.7, 47.8, 47.9, 48, 48.1, 48.2, 48.3, 48.4, 48.5, 48.6, 48.7, 48.8, 48.9, 49, 49.1, 49.2, 49.3, 49.4, 49.5, 49.6, 49.7, 49.8, 49.9, 50, 50.1, 50.2, 50.3, 50.4, 50.5, 50.6, 50.7, 50.8, 50.9, 51, 51.1, 51.2, 51.3, 51.4, 51.5, 51.6, 51.7, 51.8, 51.9, 52, 52.1, 52.2, 52.3, 52.4, 52.5, 52.6, 52.7, 52.8, 52.9, 53, 53.1, 53.2, 53.3, 53.4, 53.5, 53.6, 53.7, 53.8, 53.9, 54, 54.1, 54.2, 54.3, 54.4, 54.5, 54.6, 54.7, 54.8, 54.9, 55, 55.1, 55.2, 55.3, 55.4, 55.5, 55.6, 55.7, 55.8, 55.9, 56, 56.1, 56.2, 56.3, 56.4, 56.5, 56.6, 56.7, 56.8, 56.9, 57, 57.1, 57.2, 57.3, 57.4, 57.5, 57.6, 57.7, 57.8, 57.9, 58, 58.1, 58.2, 58.3, 58.4, 58.5, 58.6, 58.7, 58.8, 58.9, 59, 59.1, 59.2, 59.3, 59.4, 59.5, 59.6, 59.7, 59.8, 59.9, 60, 60.1, 60.2, 60.3, 60.4, 60.5, 60.6, 60.7, 60.8, 60.9, 61, 61.1, 61.2, 61.3, 61.4, 61.5, 61.6, 61.7, 61.8, 61.9, 62, 62.1, 62.2, 62.3, 62.4, 62.5, 62.6, 62.7, 62.8, 62.9, 63, 63.1, 63.2, 63.3, 63.4, 63.5, 63.6, 63.7, 63.8, 63.9, 64, 64.1, 64.2, 64.3, 64.4, 64.5, 64.6, 64.7, 64.8, 64.9, 65, 65.1, 65.2, 65.3, 65.4, 65.5, 65.6, 65.7, 65.8, 65.9, 66, 66.1, 66.2, 66.3, 66.4, 66.5, 66.6, 66.7, 66.8, 66.9, 67, 67.1, 67.2, 67.3, 67.4, 67.5, 67.6, 67.7, 67.8, 67.9, 68, 68.1, 68.2, 68.3, 68.4, 68.5, 68.6, 68.7, 68.8, 68.9, 69, 69.1, 69.2, 69.3, 69.4, 69.5, 69.6, 69.7, 69.8, 69.9, 70, 70.1, 70.2, 70.3, 70.4, 70.5, 70.6, 70.7, 70.8, 70.9, 71, 71.1, 71.2, 71.3, 71.4, 71.5, 71.6, 71.7, 71.8, 71.9, 72, 72.1, 72.2, 72.3, 72.4, 72.5, 72.6, 72.7, 72.8, 72.9, 73, 73.1, 73.2, 73.3, 73.4, 73.5, 73.6, 73.7, 73.8, 73.9, 74, 74.1, 74.2, 74.3, 74.4, 74.5, 74.6, 74.7, 74.8, 74.9, 75, 75.1, 75.2, 75.3, 75.4, 75.5, 75.6, 75.7, 75.8, 75.9, 76, 76.1, 76.2, 76.3, 76.4, 76.5, 76.6, 76.7, 76.8, 76.9, 77, 77.1, 77.2, 77.3, 77.4, 77.5, 77.6, 77.7, 77.8, 77.9, 78, 78.1, 78.2, 78.3, 78.4, 78.5, 78.6, 78.7, 78.8, 78.9, 79, 79.1, 79.2, 79.3, 79.4, 79.5, 79.6, 79.7, 79.8, 79.9, 80, 80.1, 80.2, 80.3, 80.4, 80.5, 80.6, 80.7, 80.8, 80.9, 81, 81.1, 81.2, 81.3, 81.4, 81.5, 81.6, 81.7, 81.8, 81.9, 82, 82.1, 82.2, 82.3, 82.4, 82.5, 82.6, 82.7, 82.8, 82.9, 83, 83.1, 83.2, 83.3, 83.4, 83.5, 83.6, 83.7, 83.8, 83.9, 84, 84.1, 84.2, 84.3, 84.4, 84.5, 84.6, 84.7, 84.8, 84.9, 85, 85.1, 85.2, 85.3, 85.4, 85.5, 85.6, 85.7, 85.8, 85.9, 86, 86.1, 86.2, 86.3, 86.4, 86.5, 86.6, 86.7, 86.8, 86.9, 87, 87.1, 87.2, 87.3, 87.4, 87.5, 87.6, 87.7, 87.8, 87.9, 88, 88.1, 88.2, 88.3, 88.4, 88.5, 88.6, 88.7, 88.8, 88.9, 89, 89.1, 89.2, 89.3, 89.4, 89.5, 89.6, 89.7, 89.8, 89.9, 90, 90.1, 90.2, 90.3, 90.4, 90.5, 90.6, 90.7, 90.8, 90.9, 91, 91.1, 91.2, 91.3, 91.4, 91.5, 91.6, 91.7, 91.8, 91.9, 92, 92.1, 92.2, 92.3, 92.4, 92.5, 92.6, 92.7, 92.8, 92.9, 93, 93.1, 93.2, 93.3, 93.4, 93.5, 93.6, 93.7, 93.8, 93.9, 94, 94.1, 94.2, 94.3, 94.4, 94.5, 94.6, 94.7, 94.8, 94.9, 95, 95.1, 95.2, 95.3, 95.4, 95.5, 95.6, 95.7, 95.8, 95.9, 96, 96.1, 96.2, 96.3, 96.4, 96.5, 96.6, 96.7, 96.8, 96.9, 97, 97.1, 97.2, 97.3, 97.4, 97.5, 97.6, 97.7, 97.8, 97.9, 98, 98.1, 98.2, 98.3, 98.4, 98.5, 98.6, 98.7, 98.8, 98.9, 99, 99.1, 99.2, 99.3, 99.4, 99.5, 99.6, 99.7, 99.8, 99.9, 100, 100.1, 100.2, 100.3, 100.4, 100.5, 100.6, 100.7, 100.8, 100.9, 101, 101.1, 101.2, 101.3, 101.4, 101.5, 101.6, 101.7, 101.8, 101.9, 102, 102.1, 102.2, 102.3, 102.4, 102.5, 102.6, 102.7, 102.8, 102.9, 103, 103.1, 103.2, 103.3, 103.4, 103.5, 103.6, 103.7, 103.8, 103.9, 104, 104.1, 104.2, 104.3, 104.4, 104.5, 104.6, 104.7, 104.8, 104.9, 105, 105.1, 105.2, 105.3, 105.4, 105.5, 105.6, 105.7, 105.8, 105.9, 106, 106.1, 106.2, 106.3, 106.4, 106.5, 106.6, 106.7, 106.8, 106.9, 107, 107.1, 107.2, 107.3, 107.4, 107.5, 107.6, 107.7, 107.8, 107.9, 108, 108.1, 108.2, 108.3, 108.4, 108.5, 108.6, 108.7, 108.8, 108.9, 109, 109.1, 109.2, 109.3, 109.4, 109.5, 109.6, 109.7, 109.8, 109.9, 110, 110.1, 110.2, 110.3, 110.4, 110.5, 110.6, 110.7, 110.8, 110.9, 111, 111.1, 111.2, 111.3, 111.4, 111.5, 111.6, 111.7, 111.8, 111.9, 112, 112.1, 112.2, 112.3, 112.4, 112.5, 112.6, 112.7, 112.8, 112.9, 113, 113.1, 113.2, 113.3, 113.4, 113.5, 113.6, 113.7, 113.8, 113.9, 114, 114.1, 114.2, 114.3, 114.4, 114.5, 114.6, 114.7, 114.8, 114.9, 115, 115.1, 115.2, 115.3, 115.4, 115.5, 115.6, 115.7, 115.8, 115.9, 116, 116.1, 116.2, 116.3, 116.4, 116.5, 116.6, 116.7, 116.8, 116.9, 117, 117.1, 117.2, 117.3, 117.4, 117.5, 117.6, 117.7, 117.8, 117.9, 118, 118.1, 118.2, 118.3, 118.4, 118.5, 118.6, 118.7, 118.8, 118.9, 119, 119.1, 119.2, 119.3, 119.4, 119.5, 119.6, 119.7, 119.8, 119.9, 120, 120.1, 120.2, 120.3, 120.4, 120.5, 120.6, 120.7, 120.8, 120.9, 121, 121.1, 121.2, 121.3, 121.4, 121.5, 121.6, 121.7, 121.8, 121.9, 122, 122.1, 122.2, 122.3, 122.4, 122.5, 122.6, 122.7, 122.8, 122.9, 123, 123.1, 123.2, 123.3, 123.4, 123.5, 123.6, 123.7, 123.8, 123.9, 124, 124.1, 124.2, 124.3, 124.4, 124.5, 124.6, 124.7, 124.8, 124.9, 125, 125.1, 125.2, 125.3, 125.4, 125.5, 125.6, 125.7, 125.8, 125.9, 126, 126.1, 126.2, 126.3, 126.4, 126.5, 126.6, 126.7, 126.8, 126.9, 127, 127.1, 127.2, 127.3, 127.4, 127.5, 127.6, 127.7, 127.8, 127.9, 128, 128.1, 128.2, 128.3, 128.4, 128.5, 128.6, 128.7, 128.8, 128.9, 129, 129.1, 129.2, 129.3, 129.4, 129.5, 129.6, 129.7, 129.8, 129.9, 130, 130.1, 130.2, 130.3, 130.4, 130.5, 130.6, 130.7, 130.8, 130.9, 131, 131.1, 131.2, 131.3, 131.4, 131.5, 131.6, 131.7, 131.8, 131.9, 132, 132.1, 132.2, 132.3, 132.4, 132.5, 132.6, 132.7, 132.8, 132.9, 133, 133.1, 133.2, 133.3, 133.4, 133.5, 133.6, 133.7, 133.8, 133.9, 134, 134.1, 134.2, 134.3, 134.4, 134.5, 134.6, 134.7, 134.8, 134.9, 135, 135.1, 135.2, 135.3, 135.4, 135.5, 135.6, 135.7, 135.8, 135.9, 136, 136.1, 136.2, 136.3, 136.4, 136.5, 136.6, 136.7, 136.8, 136.9, 137, 137.1, 137.2, 137.3, 137.4, 137.5, 137.6, 137.7, 137.8, 137.9, 138, 138.1, 138.2, 138.3, 138.4, 138.5, 138.6, 138.7, 138.8, 138.9, 139, 139.1, 139.2, 139.3, 139.4, 139.5, 139.6, 139.7, 139.8, 139.9, 140, 140.1, 140.2, 140.3, 140.4, 140.5, 140.6, 140.7, 140.8, 140.9, 141, 141.1, 141.2, 141.3, 141.4, 141.5, 141.6, 141.7, 141.8, 141.9, 142, 142.1, 142.2, 142.3, 142.4, 142.5, 142.6, 142.7, 142.8, 142.9, 143, 143.1, 143.2, 143.3, 143.4, 143.5, 143.6, 143.7, 143.8, 143.9, 144, 144.1, 144.2, 144.3, 144.4, 144.5, 144.6, 144.7, 144.8, 144.9, 145, 145.1, 145.2, 145.3, 145.4, 145.5, 145.6, 145.7, 145.8, 145.9, 146, 146.1, 146.2, 146.3, 146.4, 146.5, 146.6, 146.7, 146.8, 146.9, 147, 147.1, 147.2, 147.3, 147.4, 147.5, 147.6, 147.7, 147.8, 147.9, 148, 148.1, 148.2, 148.3, 148.4, 148.5, 148.6, 148.7, 148.8, 148.9, 149, 149.1, 149.2, 149.3, 149.4, 149.5, 149.6, 149.7, 149.8, 149.9, 150, 150.1, 150.2, 150.3, 150.4, 150.5, 150.6, 150.7, 150.8, 150.9, 151, 151.1, 151.2, 151.3, 151.4, 151.5, 151.6, 151.7, 151.8, 151.9, 152, 152.1, 152.2, 152.3, 152.4, 152.5, 152.6, 152.7, 152.8, 152.9, 153, 153.1, 153.2, 153.3, 153.4, 153.5, 153.6, 153.7, 153.8, 153.9, 154, 154.1, 154.2, 154.3, 154.4, 154.5, 154.6, 154.7, 154.8, 154.9, 155, 155.1, 155.2, 155.3, 155.4, 155.5, 155.6, 155.7, 155.8, 155.9, 156, 156.1, 156.2, 156.3, 156.4, 156.5, 156.6, 156.7, 156.8, 156.9, 157, 157.1, 157.2, 157.3, 157.4, 157.5, 157.6, 157.7, 157.8, 157.9, 158, 158.1, 158.2, 158.3, 158.4, 158.5, 158.6, 158.7, 158.8, 158.9, 159, 159.1, 159.2, 159.3, 159.4, 159.5, 159.6, 159.7, 159.8, 159.9, 160, 160.1, 160.2, 160.3, 160.4, 160.5, 160.6, 160.7, 160.8, 160.9, 161, 161.1, 161.2, 161.3, 161.4, 161.5, 161.6, 161.7, 161.8, 161.9, 162, 162.1, 162.2, 162.3, 162.4, 162.5, 162.6, 162.7, 162.8, 162.9, 163, 163.1, 163.2, 163.3, 163.4, 163.5, 163.6, 163.7, 163.8, 163.9, 164, 164.1, 164.2, 164.3, 164.4, 164.5, 164.6, 164.7, 164.8, 164.9, 165, 165.1, 165.2, 165.3, 165.4, 165.5, 165.6, 165.7, 165.8, 165.9, 166, 166.1, 166.2, 166.3, 166.4, 166.5, 166.6, 166.7, 166.8, 166.9, 167, 167.1, 167.2, 167.3, 167.4, 167.5, 167.6, 167.7, 167.8, 167.9, 168, 168.1, 168.2, 168.3, 168.4, 168.5, 168.6, 168.7, 168.8, 168.9, 169, 169.1, 169.2, 169.3, 169.4, 169.5, 169.6, 169.7, 169.8, 169.9, 170, 170.1, 170.2, 170.3, 170.4, 170.5, 170.6, 170.7, 170.8, 170.9, 171, 171.1, 171.2, 171.3, 171.4, 171.5, 171.6, 171.7, 171.8, 171.9, 172, 172.1, 172.2, 172.3, 172.4, 172.5, 172.6, 172.7, 172.8, 172.9, 173, 173.1, 173.2, 173.3, 173.4, 173.5, 173.6, 173.7, 173.8, 173.9, 174, 174.1, 174.2, 174.3, 174.4, 174.5, 174.6, 174.7, 174.8, 174.9, 175, 175.1, 175.2, 175.3, 175.4, 175.5, 175.6, 175.7, 175.8, 175.9, 176, 176.1, 176.2, 176.3, 176.4, 176.5, 176.6, 176.7, 176.8, 176.9, 177, 177.1, 177.2, 177.3, 177.4, 177.5, 177.6, 177.7, 177.8, 177.9, 178, 178.1, 178.2, 178.3, 178.4, 178.5, 178.6, 178.7, 178.8, 178.9, 179, 179.1, 179.2, 179.3, 179.4, 179.5, 179.6, 179.7, 179.8, 179.9, 180, 180.1, 180.2, 180.3, 180.4, 180.5, 180.6, 180.7, 180.8, 180.9, 181, 181.1, 181.2, 181.3, 181.4, 181.5, 181.6, 181.7, 181.8, 181.9, 182, 182.1, 182.2, 182.3, 182.4, 182.5, 182.6, 182.7, 182.8, 182.9, 183, 183.1, 183.2, 183.3, 183.4, 183.5, 183.6, 183.7, 183.8, 183.9, 184, 184.1, 184.2, 184.3, 184.4, 184.5, 184.6, 184.7, 184.8, 184.9, 185, 185.1, 185.2, 185.3, 185.4, 185.5, 185.6, 185.7, 185.8, 185.9, 186, 186.1, 186.2, 186.3, 186.4, 186.5, 186.6, 186.7, 186.8, 186.9, 187, 187.1, 187.2, 187.3, 187.4, 187.5, 187.6, 187.7, 187.8, 187.9, 188, 188.1, 188.2, 188.3, 188.4, 188.5, 188.6, 188.7, 188.8, 188.9, 189, 189.1, 189.2, 189.3, 189.4, 189.5, 189.6, 189.7, 189.8, 189.9, 190, 190.1, 190.2, 190.3, 190.4, 190.5, 190.6, 190.7, 190.8, 190.9, 191, 191.1, 191.2, 191.3, 191.4, 191.5, 191.6, 191.7, 191.8, 191.9, 192, 192.1, 192.2, 192.3, 192.4, 192.5, 192.6, 192.7, 192.8, 192.9, 193, 193.1, 193.2, 193.3, 193.4, 193.5, 193.6, 193.7, 193.8, 193.9, 194, 194.1, 194.2, 194.3, 194.4, 194.5, 194.6, 194.7, 194.8, 194.9, 195, 195.1, 195.2, 195.3, 195.4, 195.5, 195.6, 195.7, 195.8, 195.9, 196, 196.1, 196.2, 196.3, 196.4, 196.5, 196.6, 196.7, 196.8, 196.9, 197, 197.1, 197.2, 197.3, 197.4, 197.5, 197.6, 197.7, 197.8, 197.9, 198, 198.1, 198.2, 198.3, 198.4, 198.5, 198.6, 198.7, 198.8, 198.9, 199, 199.1, 199.2, 199.3, 199.4, 199.5, 199.6, 199.7, 199.8, 199.9, or 200 mg/g.

In some embodiments, the water dispersible nanoemulsion comprising one or more cannabinoids provides a liquid beverage concentrate composition for the preparation of liquid beverages comprising one or more cannabinoids.

In one embodiment, the liquid beverage concentrate composition comprises;
a) an extract of a cannabis plant, preferably cannabis resin;
b) an essential oil, preferably orange oil;
c) a medium chain triglyceride, preferably glycerol tricaproate;
d) a poly(ethylene glycol), preferably PEG 35;
e) a combination of α-tocopherol, α-tocopherol acetate and α-tocopherol succinate, preferably in a ratio of 2:1:1; and
f) water.

In one embodiment, the liquid beverage concentrate composition further comprises;
g) a modified starch, preferably corn starch;
h) a polysaccharide, preferably maltodextrin;
i) an antioxidant, preferably vitamin C;
j) a food acid, preferably citric acid;
k) an inorganic salt, preferably sodium bicarbonate;
l) an organic slat, preferably magnesium citrate;
m) a plant gum, preferably guar gum and/or xanthan gum; and optionally
n) a soluble fibre.

In some embodiments, there is provided a drinkable beverage composition prepared from the water dispersible nanoemulsion or the liquid beverage concentrate composition comprising one or more cannabinoids.

In some embodiments, there is provided a drinkable beverage composition prepared from the water soluble powder comprising one or more cannabinoids.

In some embodiments, the one or more cannabinoids may be present in the beverage composition in respective amounts of approximately 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 mg per serve of beverage.

In some embodiments, the one or more cannabinoids may be present in the beverage composition in respective amounts of from about 2.5 mg to about 30 mg, in particular from about 5 mg to about 15 mg per serve of beverage.

For example, THC may be present in the beverage composition in an amount of 10 mg and CBD may be present in the beverage composition in an amount of 5 mg per serve of beverage.

In some embodiments, the one or more cannabinoids may be present in the beverage composition in respective amounts of from about 10 mg/L to about 5000 mg/L; or from about 25 mg/L to about 300 mg/L; or THC is present in the beverage in an amount of 50 to 150 mg/L and CBD is present in the beverage in an amount of 25 to 75 mg/L; or THC is present in the beverage in an amount of 100 mg/L and CBD is present in the beverage in an amount of 50 mg/L.

In one embodiment, the nanoemulsion or powder comprises surfactant in an amount of approximately 1, 2, 3, 4, 5, 6, 7, 8, 9, 10, 11, 12, 13, 14, 15, 16, 17, 18, 19, or 20 wt %.

In one embodiment, the nanoemulsion or powder comprises from 4-10 wt % surfactant. The surfactant may be a non-ionic surfactant, an ionic surfactant, or a zwitterionic surfactant. The surfactant may be any surfactant suitable for use in food products. For example, without limitation, the surfactant may be any surfactant referred to in the published review; Iva Kralova & Johan Sjöblom (2009) Surfactants Used in Food Industry: A Review, Journal of Dispersion Science and Technology, 30:9, 1363-1383, the contents of which are hereby incorporated in their entirety.

In one embodiment, the drinkable liquid beverage comprises water, carbonated water, flavoured carbonated water, juice of one or more fruits and/or vegetables, or a mixture thereof. The aqueous phase of the nanoemulsion may be the same as or different to the drinkable liquid beverage.

In one embodiment, the nanoemulsion or powder may further comprise a co-surfactant. The co-surfactant may be, without limitation, a short-chain amine, a short-chain alcohol, a short-chain polyamine, a short-chain polyalcohol, a short-chain aminoalcohol, propylene glycol, ethylene glycol, glycerine, or a mixture of any of the aforesaid.

In some embodiments, the beverage composition may further comprise one or more additives comprising a taste modulator, an antioxidant, a colourant, a micronutrient, or a mixture thereof. The one or more additives may be soluble in the drinkable liquid. Additionally or alternatively, the nanoemulsion or powder may further comprise the one or more additives.

The disclosure also provides a beverage concentrate composition which is arranged, in use, to be mixed with a drinkable liquid to prepare a beverage composition. The beverage concentrate composition may be a liquid or nanoemulsion that can be diluted by or dispersed in the drinkable liquid or a particulate or powder material that is soluble or dispersible in the drinkable liquid.

In some embodiments, the liquid beverage concentrate composition comprises a nanoemulsion comprising one or more cannabinoids in an oil phase, a surfactant, and an aqueous phase.

In some embodiments, the particulate or powder material comprises a mixture of said nanoemulsion and an encapsulation agent, whereby the mixture is dried to produce a water soluble particulate or powder material. As used herein, the terms “encapsulation agent” and “encapsulation excipient” are used interchangeably. The encapsulation agent may be any suitable film-forming and GRAS substance soluble in water, or a mixture of water and ethanol. For example, without limitation, the encapsulation agent may be a polysaccharide (such as maltodextrin), starch, cellulose, cellulose derivative, a polyvinyl alcohol, a gelatin, a carageenan, a hydrogel, an alginate or alginate salt, an edible polymer, a protein (such as whey protein or casein), or an ionic salt.

In some embodiments, there is provided a method of preparing a beverage which comprises mixing a serving of a beverage concentrate composition as defined above in a drinkable liquid, wherein a serving comprises 1, 2, 3, 4, 5, 6, 7, 8, 9, 10, 11, 12, 13, 14, 15, 16, 17, 18, 19,20, 21, 22, 23,24,25, 26, 27,28, 29, 30, 31,32,33, 34, 35, 36, 37, 38, 39, 40, 41, 42, 43, 44, 45, 46, 47, 48, 49, or 50 mL of nanoemulsion concentrate, per 100 mL of drinkable liquid.

In some embodiments, there is provided a method of preparing a beverage which comprises mixing a serving of a beverage concentrate composition as defined above in a drinkable liquid, wherein a serving comprises 1, 2, 3, 4, 5, 6, 7, 8, 9, 10, 11, 12, 13, 14, 15, 16, 17, 18, 19,20,21,22,23, 24,25,26,27,28,29,30,31,32,33, 34, 35, 36, 37, 38, 39, 40, 41, 42, 43, 44, 45, 46, 47, 48, 49, or 50 g of particulate or powder concentrate composition per 100 mL of drinkable liquid.

In some embodiments, there is provided a method of preparing a beverage which comprises mixing a serving of a beverage concentrate composition as defined above in a drinkable liquid, wherein a serving comprises from 10 to 15 mL of nanoemulsion concentrate, or when encapsulated 1-10 g of particulate or powder concentrate composition per 100 mL of drinkable liquid.

Another aspect of the disclosure provides a method of manufacturing a beverage composition as defined above, said method comprising subjecting a mixture of an aqueous phase, a surfactant and an oil phase to a high energy or a low energy emulsification technique for a sufficient time to produce a nanoemulsion, wherein the aqueous phase comprises a drinkable liquid and the oil phase comprises one or more cannabinoids.

Another aspect of the disclosure provides an alternative method of manufacturing a beverage composition as defined above, said method comprising:
a) subjecting a mixture of an aqueous phase, a surfactant and an oil phase to a high energy or a low energy emulsification technique for a sufficient time to produce a nanoemulsion, wherein the oil phase comprises one or more cannabinoids and the aqueous phase is miscible with a drinkable liquid; and
b) mixing the nanoemulsion with the drinkable liquid.

Another aspect of the disclosure provides an alternative method of manufacturing a beverage composition as defined above, said method comprising:
a) subjecting a mixture of an aqueous phase, a surfactant and an oil phase to a high energy or a low energy emulsification technique for a sufficient time to produce a nanoemulsion, wherein the oil phase comprises one or more cannabinoids and the aqueous phase is miscible with a drinkable liquid;
b) adding an encapsulation agent to the nanoemulsion;
c) drying the nanoemulsion in the presence of the added encapsulation agent to provide a particulate or powder composition and;
d) mixing the particulate or powder composition with the drinkable liquid.

In one embodiment, the present disclosure provides a liquid beverage concentrate composition, comprising a nanoemulsion, wherein the nanoemulsion comprises one or more cannabinoids in an oil phase, at least one surfactant, and an aqueous phase.

In some embodiments, the one or more cannabinoids of the liquid beverage concentrate composition is selected from the group consisting of anandamide, 2-arachidonoylglycerol, cannabichromene (CBC), cannabichromenic acid (CBCA), cannabichromevarin (CBCV), cannabichromevarinic acid (CBCVA), cannabidiol (CBD), cannabidiolic acid (CBDA), cannabidivarin (CBDV), cannabidivarinic acid (CBDVA), cannabielsoin (CBE), cannabicyclol (CBL), cannabinodiol (CBND), cannabigerol (CBG), cannabigerolic acid (CBGA), cannabigerovarin (CBGV), cannabigerovarinic acid (CBGVA), cannabinol (CBN), cannabinolic acid (CBNA), cannabitriol (CBT), delta-8-tetrahydrocanninol, delta-8-tetrahydrocannabinolic acid, delta-9-tetrahydrocannabinol (THC), delta-9-tetrahydrocannabinolic acid (THCA), delta-9-tetrahydrocannabivarin (THCV), delta-9-tetrahydrocannabivarinic acid (THCVA), 11-nor-9-carboxy-delta-9-tetrahydrocannabinol (THCCOOCH), 11-nor-9-carboxy-delta-8-tetrahydrocannabinol, 11-hydroxy-delta-8-tetrahydrocannabinol, and 11-hydroxy-delta-9-tetrahydrocannabinol, dimethyl heptylpentyl cannabidiol (DMHP-CBD), 6,12-dihydro-6-hydroxy-cannabidiol, (3S,4R)-7-hydroxy-.DELTA.6-tetrahydrocannabinol homologs and derivatives, (+)-4-[4-DMH-2,6-diacetoxy-phenyl]-2-carboxy-6,6-dimethylbicyclo[3.1.1]hept-2-en, and other 4-phenylpinene derivatives, and cannabidiol (−)(CBD) analogs such as (−)CBD-monomethylether, (−)CBD dimethyl ether; (−)CBD diacetate; (−)3′-acetyl-CBD monoacetate, cannabinol propyl variant (CBNV), nabilone, synthetic cannabinoids, and cannabinoid analogues, including derivatives, analogues, stereoisomers, racemates, scalemates and pharmaceutically acceptable salts of any of the aforementioned cannabinoids.

In some embodiments, the one or more cannabinoids of the liquid beverage concentrate composition comprise;
a) an extract of a cannabis plant; or
b) an extract of a cannabis flower; or
c) an entourage of cannabinoids; or
d) an entourage of cannabinoids comprising cannabichromene (CBC), cannabidiol (CBD), cannabidiolic acid (CBDA), cannabidivarin (CBDV), cannabicyclol (CBL), cannabigerol (CBG), cannabinol (CBN), delta-9-tetrahydrocannabinol (THC), delta-9-tetrahydrocannabinolic acid (THCA), and delta-9-tetrahydrocannabivarin (THCV); or
e) THC; or
f) CBD; or
g) THC and CBD.

In some embodiments, the nanoemulsion of the liquid beverage concentrate composition comprises from 4-10 wt % surfactant.

In some embodiments, the nanoemulsion of the liquid beverage concentrate composition comprises a co-surfactant.

In some embodiments, the oil phase of the liquid beverage concentrate composition comprises an oil selected from the group consisting of; an edible oil, a paraffin oil, an essential oil, orange oil;
a short chain triglyceride, selected from the group consisting of; glycerol triacetate, glycerol tripropionate, glycerol tributyrate, glycerol trivalerate, or a compound of Formula (I);

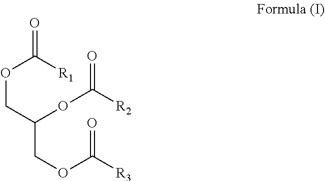

wherein R₁, R₂ and R₃ are independently selected from the group consisting of methyl, ethyl, propyl, isopropyl, cyclopropyl, n-butyl, sec-butyl, tert-butyl, cyclobutyl, ethenyl, propenyl, isopropenyl, cyclopropenyl, n-butenyl, sec-butenyl, cyclobutenyl, n-butadienyl and sec-butadienyl, including mixtures of any two or more of the aforesaid short chain triglycerides; or
a medium chain triglyceride, selected from the group consisting of; glycerol tricaproate, glycerol tricaprylate, glycerol tricaprate, glycerol trilaurate, or a compound of Formula (II);

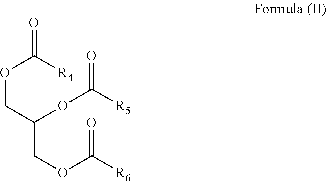

wherein R₄, R₅ and R₆ are independently selected from the group consisting of C₅₋₁₁ alkyl, C₅₋₁₁ alkenyl, C₅₋₁₁ alkadienyl, C₆₋₁₁ alkatrienyl, C₈₋₁₁ alkatetraenyl, and C₁₀₋₁₁ alkapentaenyl, wherein said alkyl, alkenyl, alkadienyl, alkatrienyl, alkatetraenyl, and alkapentaenyl substituents may each be linear, branched or cyclic, including mixtures of any two or more of the aforesaid medium chain triglycerides;
including mixtures of any of the aforesaid oils.

In some embodiments, the liquid beverage concentrate composition further comprises one or more additives selected from the group consisting of a taste modulator, an antioxidant, a colourant, a micronutrient, or a mixture of any of the aforementioned additives. In some embodiments, the one or more additives are soluble in the aqueous phase, and/or one or more additives are soluble in the oil phase.

In some embodiments, the liquid beverage concentrate composition further comprises one or more encapsulation agents or encapsulation excipients, soluble in the aqueous phase. In preferred embodiments, the one or more encapsulation agents or encapsulation excipients is selected from the group consisting of a starch, cellulose, a cellulose derivative, a polyvinyl alcohol, a gelatin, a carageenan, a hydrogel, an alginate, an alginate salt, an edible polymer, a protein, whey protein, casein, an ionic salt and sodium bicarbonate.

In one embodiment, the disclosure herein provides a process for the preparation of the liquid beverage concentrate embodiments of the invention, comprising subjecting a mixture of an aqueous phase, a surfactant and an oil phase to a high energy or a low energy emulsification technique for a sufficient period to produce a nanoemulsion, wherein the oil phase comprises one or more cannabinoids, and the aqueous phase comprises a drinkable liquid.

In one embodiment, the disclosure herein provides a process for the preparation of a particulate or powder beverage concentrate composition comprising one or more cannabinoids, said process comprising;
a) subjecting a mixture of an aqueous phase, a surfactant and an oil phase to a high energy or a low energy emulsification technique for a sufficient time to produce a liquid beverage concentrate composition comprising a nanoemulsion, wherein the oil phase comprises one or more cannabinoids;
b) adding an encapsulation agent to the nanoemulsion; and
c) drying the nanoemulsion in the presence of the added encapsulation agent to provide a particulate or powder composition.

In one embodiment, the disclosure herein provides a process for the preparation of a particulate or powder beverage concentrate composition comprising drying a liquid beverage concentrate composition comprising a nanoemulsion in accordance with the present invention, wherein the nanoemulsion comprises one or more encapsulation agents or encapsulation excipients, soluble in the aqueous phase.

In some embodiments, the step of drying the nanoemulsion to provide the particulate or powder beverage concentrate composition comprises spray drying.

In some embodiments, particulate or powder beverage concentrate composition further comprises one or more additives selected from the group consisting of a taste modulator, an antioxidant, a colourant, a micronutrient, or a mixture of any of the aforementioned additives, wherein the one or more additives is in a particulate or powdered form that is water soluble or dispersible.

In one embodiment, the disclosure herein provides a method of preparing a beverage containing one or more cannabinoids, said method comprising diluting or dispersing a predetermined amount of the liquid beverage concentrate composition of the invention, or dissolving or dispersing a predetermined amount of the particulate or powder beverage concentrate composition of the invention, in a volume of a drinkable liquid, to produce a serving of beverage.

In one embodiment, the disclosure herein provides a beverage, comprising the liquid beverage concentrate composition of the invention, diluted or dispersed in a drinkable liquid, or the particulate or powder beverage concentrate of the invention, dissolved or dispersed in a drinkable liquid.

In some embodiments, the one or more cannabinoids are present in the beverage in respective amounts of from about 1 mg to about 500 mg, preferably in respective amounts of from about 2.5 mg to about 30 mg, per serving of the beverage.

In some embodiments, the one or more cannabinoids are present in the beverage in respective amounts of from about 10 mg/L to about 5000 mg/L, preferably in respective amounts of from about 25 mg/L to about 300 mg/L, of the beverage.

In some embodiments, THC is present in the beverage composition in an amount of 50 to 150 mg/L and CBD is present in the beverage composition in an amount of 25 to 75 mg/L, of the beverage.

In some embodiments, THC is present in the beverage composition in an amount of 100 mg/L and CBD is present in the beverage composition in an amount of 50 mg/L, of the beverage.

In some embodiments, THC is present in the beverage composition in an amount of 10 mg and CBD is present in the beverage composition in an amount of 5 mg, per serving of the beverage.

In some embodiments, a serving of the beverage is a 100 ml serving.

In some embodiments, the drinkable liquid comprises water, carbonated water, flavoured carbonated water, juice of one or more fruits and/or vegetables, or a mixture thereof.

In some embodiments, the liquid beverage concentrate composition, or the beverage, exhibit transparency or translucency stability, and/or chemical stability, and/or colour stability, for a period of at least 12 months.

In some embodiments, transparency or translucency stability, is determined by evaluating, for a period of at least 12 months, transparency or translucency with the naked eye, and/or by measuring for a period of at least 12 months, transparency or translucency using a turbidimeter, and/or by measuring for a period of at least 12 months, transparency or translucency via visible absorbance spectrometry.

In some embodiments, the particulate or powder beverage concentrate composition exhibits chemical stability, and/or colour stability, for a period of at least 12 months.

In some embodiments, chemical stability, is determined by analysing, for a period of at least 12 months, the degradation of one or more cannabinoid compounds via LCMSMS, GCMS, HPLCMS, or GCMSMS.

In some embodiments, colour stability, is determined by evaluating, for a period of at least 12 months, consistently maintained identity of colour in accordance with CIE 1976 L*a*b* (CIELAB), and/or HunterLab L,a,b and/or AS 2700 colour scale systems.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 Is a diagram of the proposed self-assembled droplet formed in the preparation of the nanoemulsion process of the present invention. Without wishing to be bound by theory, it is believed that surfactant molecules self-assemble into a nanodroplet as depicted, with the aqueous phase of the nanoemulsion on the outside of the droplet, and the lipophilic phase comprising Medium Chain Triglycerides (MCTs) and one or more cannabinoids on the inside of the nanodroplet.

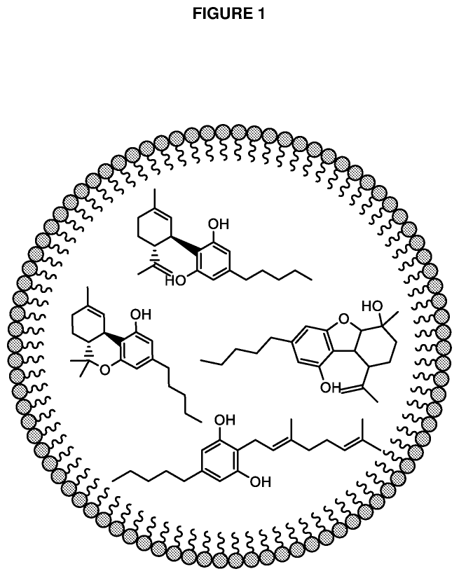

FIG. 2 Is a schematic flow chart depicting some embodiments of the process of the present invention. Fruit derived flavouring and/or colouring agents and one or more cannabinoids or cannabis extracts in one or more MCT carriers are combined with one or more surfactants and one or more antioxidants, then ultrasonicated in an aqueous phase to produce nanoemulsion concentrates in accordance with the invention. The nanoemulsion concentrates may be used directly to produce beverages via dilution or dispersal in a drinkable liquid. Optionally the nanoemulsion concentrates may be further treated with an encapsulation agent or encapsulation excipient, followed by spray drying to produce the particulate or powder concentrates in accordance with the invention. The particulate or powder concentrates may be used directly to produce beverages via dissolution or dispersal in a drinkable liquid, optionally with the addition of additives such as stabilises, buffers, flavouring agents and/or sweeteners.

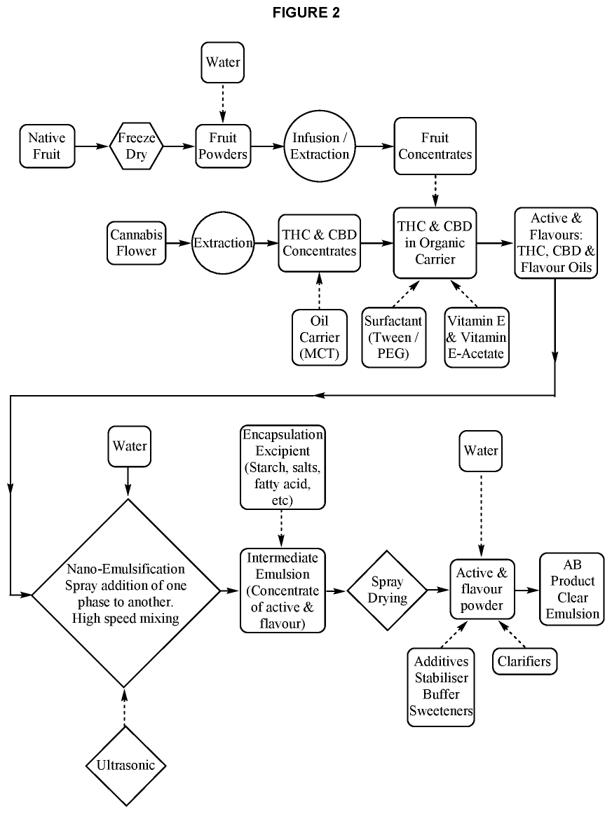

FIG. 3 Is a plot of stability data in pg/ml of an entourage of cannabinoids [cannabichromene (CBC), cannabidiol (CBD), cannabidiolic acid (CBDA), cannabidivarin (CBDV), cannabicyclol (CBL), cannabigerol (CBG), cannabinol (CBN), delta-9-tetrahydrocannabinol (THC), delta-9-tetrahydrocannabinolic acid (THCA), and delta-9-tetrahydrocannabivarin (THCV)], in a beverage embodiment of the invention (AB White), stored at 8° C., measured via Liquid Chromatography-tandem Mass Spectrometry (LCMSMS) over a period of 570 days. The data shows some slight degradation of CBD after 480 days, and a high degree of stability for THC and the remaining cannabinoids in the entourage over the entire period.

FIG. 4 Is a plot of stability data in pg/ml of an entourage of cannabinoids [cannabichromene (CBC), cannabidiol (CBD), cannabidiolic acid (CBDA), cannabidivarin (CBDV), cannabicyclol (CBL), cannabigerol (CBG), cannabinol (CBN), delta-9-tetrahydrocannabinol (THC), delta-9-tetrahydrocannabinolic acid (THCA), and delta-9-tetrahydrocannabivarin (THCV)], in a beverage embodiment of the invention (AB Red), stored at 8° C., measured via Liquid Chromatography-tandem Mass Spectrometry (LCMSMS) over a period of 570 days. The data shows some slight degradation of CBD after 420 days, and a high degree of stability for THC and the remaining cannabinoids in the entourage over the entire period.

FIG. 5 Is a plot of stability data in pg/ml of Vitamin E antioxidants (a-Tocopherol, a-Tocopheryl acetate and a-Tocopheryl succinate), in a beverage embodiment of the invention (AB White), stored at 8° C., measured via Liquid Chromatography-tandem Mass Spectrometry (LCMSMS) over a period of 570 days. The data shows no significant losses of Vitamin E until after 390 days.

FIG. 6 Is a plot of stability data in pg/ml of Vitamin E antioxidants (a-Tocopherol, a-Tocopheryl acetate and a-Tocopheryl succinate), in a beverage embodiment of the invention (AB Red), stored at 8° C., measured via Liquid Chromatography-tandem Mass Spectrometry (LCMSMS) over a period of 570 days. The data shows no significant losses of Vitamin E until after 390 days.

FIG. 7 Is a plot of stability data in pg/ml of an entourage of cannabinoids [cannabichromene (CBC), cannabidiol (CBD), cannabidiolic acid (CBDA), cannabidivarin (CBDV), cannabicyclol (CBL), cannabigerol (CBG), cannabinol (CBN), delta-9-tetrahydrocannabinol (THC), delta-9-tetrahydrocannabinolic acid (THCA), and delta-9-tetrahydrocannabivarin (THCV)], in a beverage embodiment of the invention (AB White), stored at room temperature (21-25° C.), measured via Liquid Chromatography-tandem Mass Spectrometry (LCMSMS) over a period of 570 days. The data shows some slight degradation of CBD after 390 days, and a high degree of stability for THC and the remaining cannabinoids in the entourage over the entire period.

FIG. 8 Is a plot of stability data in pg/ml of an entourage of cannabinoids [cannabichromene (CBC), cannabidiol (CBD), cannabidiolic acid (CBDA), cannabidivarin (CBDV), cannabicyclol (CBL), cannabigerol (CBG), cannabinol (CBN), delta-9-tetrahydrocannabinol (THC), delta-9-tetrahydrocannabinolic acid (THCA), and delta-9-tetrahydrocannabivarin (THCV)], in a beverage embodiment of the invention (AB Red), stored at room temperature (21-25° C.), measured via Liquid Chromatography-tandem Mass Spectrometry (LCMSMS) over a period of 570 days. The data shows some slight degradation of CBD after 390 days, and a high degree of stability for THC and the remaining cannabinoids in the entourage over the entire period.

FIG. 9 Is a plot of stability data in μg/ml of Vitamin E antioxidants (a-Tocopherol, a-Tocopheryl acetate and a-Tocopheryl succinate), in a beverage embodiment of the invention (AB White), stored at room temperature (21-25° C.), measured via Liquid Chromatography-tandem Mass Spectrometry (LCMSMS) over a period of 570 days. The data shows no significant losses of Vitamin E until after 360 days.

FIG. 10 Is a plot of stability data in μg/ml of Vitamin E antioxidants (α-Tocopherol, α-Tocopheryl acetate and α-Tocopheryl succinate), in a beverage embodiment of the invention (AB Red), stored at room temperature (21-25° C.), measured via Liquid Chromatography-tandem Mass Spectrometry (LCMSMS) over a period of 570 days. The data shows no significant losses of Vitamin E until after 360 days.

FIG. 11 Is a diagram depicting the encapsulation process of the proposed self-assembled droplet formed in the preparation of the nanoemulsion process of the present invention (left) to produce the particulate or powder concentrates in accordance with the present invention (right). An encapsulation agent or encapsulation excipient is added to the nanoemulsion concentrate composition, prior to drying via lyophilisation, spray drying or any other appropriate means, to form the encapsulated particulate or powder composition (right). It should be pointed out that the particulate or powder composition diagram (right) is not intended to depict the precise structure of the particulate or powder composition. Rather, it is merely intended to show that the encapsulated particles contain the components depicted, including surfactant molecules, Medium Chain Triglycerides (MCTs) and one or more cannabinoids present in the interior of the nanodroplet precursor to the particulate or powder composition. Once formed, the encapsulated particulate or powder concentrate composition may be dissolved or redispersed in a drinkable aqueous phase, and self-assemble into one or more nanodroplets (left), to produce a beverage in accordance with the present invention.

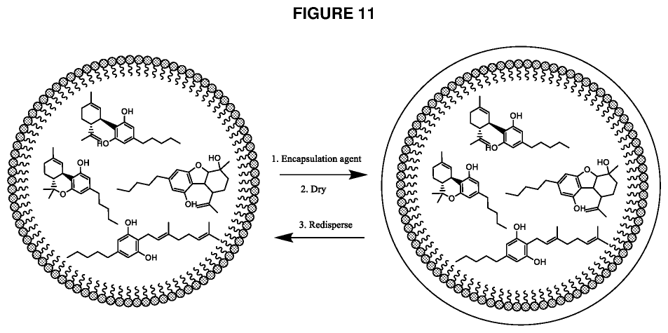

FIG. 12 Is a plot of stability data in mg/g of an entourage of cannabinoids [cannabichromene (CBC), cannabidiol (CBD), cannabidiolic acid (CBDA), cannabidivarin (CBDV), cannabicyclol (CBL), cannabigerol (CBG), cannabinol (CBN), delta-9-tetrahydrocannabinol (THC), delta-9-tetrahydrocannabinolic acid (THCA), and delta-9-tetrahydrocannabivarin (THCV)], in a particulate or powder concentrate composition of the invention, stored at room temperature (21-25° C.), measured via Liquid Chromatography-tandem Mass Spectrometry (LCMSMS) over a period of 570 days. The data shows no significant degradation of CBD, THC, or any of the remaining cannabinoids in the entourage over the entire period.

FIG. 13 Is a plot of stability data in mg/g of Vitamin E antioxidants (α-Tocopherol, α-Tocopheryl acetate and α-Tocopheryl succinate), in a particulate or powder concentrate composition of the invention, stored at room temperature (21-25° C.), measured via Liquid Chromatography-tandem Mass Spectrometry (LCMSMS) over a period of 570 days. The data shows no significant losses of Vitamin E over the entire period.

### DESCRIPTION OF EMBODIMENTS

The disclosure relates to water dispersible cannabinoid compositions suitable for the preparation of beverages, in particular beverages containing one or more cannabinoids, beverage concentrates and methods of preparing same.

### General Terms

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Each example of the present disclosure described herein is to be applied mutatis mutandis to each and every other example unless specifically stated otherwise. The present disclosure is not to be limited in scope by the specific examples described herein, which are intended for the purpose of exemplification only. Functionally-equivalent products, compositions and methods are clearly within the scope of the disclosure as described herein.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The terms “about” and “approximately” are used interchangeably, and, as used herein mean within 5%, and more preferably within 1%, of a given value or range. For example, “about 3.7%” means from 3.5 to 3.9%, preferably from 3.66 to 3.74%. When the term “about” is associated with a range of values, e.g., “about X % to Y %”, the term “about” is intended to modify both the lower (X) and upper (Y) values of the recited range. For example, “about 20% to 40%” is equivalent to “about 20% to about 40%”.

All percentages by weight in the compositions are percentages by weight with respect to the whole composition, unless specified otherwise.

### Specific Terms

The term ‘emulsion’ refers to a fluid colloidal system in which droplets of a first liquid is dispersed in a second liquid, wherein the first and second liquids are immiscible. Where the continuous phase of the emulsion is an aqueous solution, the emulsion is denoted as an oil-in-water (O/W) emulsion. The term ‘nanoemulsion’ as used herein refers to an oil-in-water (O/W) emulsion which has a droplet size up to 200 nm.

The term ‘surfactant’ refers to compounds having an amphiphilic structure which gives them a specific affinity for oil/water-type interfaces which enables them to reduce the free energy of these interfaces and to stabilise the dispersed systems.

The term ‘co-surfactant’ as used herein refers to a surfactant acting with another surfactant to further reduce the energy of the interface.

The term “lecithin” refers to phosphatidylcholine, i.e. a lipid formed from a choline, a phosphate, a glycerol and two fatty acids. More broadly, it includes phospholipids extracted from living sources, of plant or animal origin, as long as they primarily consist of phosphatidylcholine. These lecithins generally consist of mixtures of lecithins carrying different fatty acids.

The expression ‘generally recognised as safe’ or ‘GRAS’ as used herein has the same meaning as the United States Food and Drug Administration (FDA) designation and refers to a chemical or substance which is exempted from the usual Federal Food, Drug, and Cosmetic Act (FFDCA) food additive tolerance requirements because it is considered to be safe by experts when added to food.

‘The term ‘food grade’ as used herein refers to a material that is safe for human consumption.

As used herein, the term “encapsulated” and variations of the term such as “encapsulation” and “encapsulating” includes microencapsulation or nanoencapsulation, and refers to a process in which tiny particles or droplets are surrounded by a coating to give small capsule. In its simplest and idealised form, a microcapsule is a small sphere comprising a near-uniform wall enclosing some material. In reality, microcapsules are rarely spherical and may assume any three-dimensional shape. The enclosed material in the microcapsule is referred to as the core, internal phase, or fill, whereas the wall is sometimes called a shell, coating, or membrane. Some materials like lipids and polymers, such as alginate, may be used as a mixture to trap the material of interest inside. Most microcapsules have pores with diameters between a few nanometers and a few micrometers. The coating materials generally used for coating include alkyl celluloses, polyvinyl alcohols, gelatins, alginate salts, and hydrogels. Many microcapsules however bear little resemblance to simple spheres. The core may be a crystal, a jagged adsorbent particle, or a liquid, and the shell can be facilitated via formation of an emulsion, a hydrogel, a micelle, a liposome, a Pickering emulsion, a suspension of solids, or a suspension of smaller microcapsules. The microcapsule even may have multiple walls.

As used herein, the terms “encapsulation agent” and “encapsulation excipient” are used interchangeably, and refer to any suitable film-forming and GRAS substance soluble in water, or a mixture of water and ethanol. For example, without limitation, the encapsulation agent may be a starch, a polysaccharide, cellulose, cellulose derivative, a polyvinyl alcohol, a gelatin, a carageenan, a hydrogel, an alginate or alginate salt, an edible polymer, a protein (such as whey protein or casein), or an ionic salt, such as, without limitation, sodium bicarbonate.

### Beverage Composition

Various embodiments of the beverage composition as described herein comprise a nanoemulsion dispersed in a drinkable liquid, wherein the nanoemulsion comprises one or more cannabinoids in an oil phase, a surfactant and an aqueous phase.

It will be appreciated that the beverage compositions, the nanoemulsions, and the particulate or powder compositions as described herein are formulated to be safe for human consumption. Accordingly, the components of the nanoemulsions and particulate or powder compositions as will be described, are generally regarded as safe (GRAS), edible or, food grade or provided in amounts in the beverage composition which are safe for human consumption.

The drinkable liquid may be any liquid safe for human consumption. Suitable examples of the drinkable liquid may be still water, carbonated water, flavoured carbonated water such as tonic water, ginger ale or ginger beer, or juice of one or more fruits and/or vegetables. Preferably, the drinkable liquid is a cold liquid. As used in the specification, the term “cold liquid” or “cold drinkable liquid” means refrigerated liquid, i.e. a liquid, such as water, at a temperature of less than 10° C. A typical serving size of the beverage composition may be from 100 ml to 500 ml of drinkable liquid, although it is envisaged that the beverage composition may also be consumed in smaller volumes or diluted with a further volume of drinkable liquid.

Cannabinoids are a class of organic chemical compounds that act on cannabinoid receptors, also known as the endocannabinoid system in cells that alter neurotransmitter release in the brain. Phytocannabinoids are naturally occurring and are produced in the cannabis plant. Synthetic cannabinoids are manufactured artificially.

The one or more cannabinoids that may be contained in the beverage composition may be selected from the group comprising anandamide, 2-arachidonoylglycerol, cannabichromene (CBC), cannabichromenic acid (CBCA), cannabichromevarin (CBCV), cannabichromevarinic acid (CBCVA), cannabidiol (CBD), cannabidiolic acid (CBDA), cannabidivarin (CBDV), cannabidivarinic acid (CBDVA), cannabielsoin (CBE), cannabicyclol (CBL), cannabinodiol (CBND), cannabigerol (CBG), cannabigerolic acid (CBGA), cannabigerovarin (CBGV), cannabigerovarinic acid (CBGVA), cannabinol (CBN), cannabinolic acid (CBNA), cannabitriol (CBT), delta-8-tetrahydrocanninol, delta-8-tetrahydrocannabinolic acid, delta-9-tetrahydrocannabinol (THC), delta-9-tetrahydrocannabinolic acid (THCA), delta-9-tetrahydrocannabivarin (THCV), delta-9-tetrahydrocannabivarinic acid (THCVA), 11-nor-9-carboxy-delta-9-tetrahydrocannabinol (THCCOOCH), 11-nor-9-carboxy-delta-8-tetrahydrocannabinol, 11-hydroxy-delta-8-tetrahydrocannabinol, and 11-hydroxy-delta-9-tetrahydrocannabinol, dimethyl heptylpentyl cannabidiol (DMHP-CBD), 6,12-dihydro-6-hydroxy-cannabidiol, (3S,4R)-7-hydroxy-.DELTA.6-tetrahydrocannabinol homologs and derivatives, (+)-4-[4-DMH-2,6-diacetoxy-phenyl]-2-carboxy-6,6-dimethylbicyclo[3.1.1]hept-2-en, and other 4-phenylpinene derivatives, and cannabidiol (−)(CBD) analogs such as (−)CBD-monomethylether, (−)CBD dimethyl ether; (−)CBD diacetate; (−)3′-acetyl-CBD monoacetate, cannabinol propyl variant (CBNV), nabilone, synthetic cannabinoids, and cannabinoid analogues, including derivatives, analogues, stereoisomers, racemates, scalemates and pharmaceutically acceptable salts of any of the aforementioned cannabinoids.

In particular beverage compositions, the cannabinoid may comprise THC. THC is a psychoactive agent that is mostly responsible for the gentle ‘high’ associated with consumption. In other beverage compositions the cannabinoids may be THC and CBD. CBD is a non-psychotropic agent and there is some suggestion that CBD may counteract or modify the psychoactivity of THC. In further beverage compositions the cannabinoids may be an entourage of cannabinoid compounds entourage of cannabinoids derived from a cannabis plant, cannabis extract, cannabis oil, or cannabis resin. There is some suggestion that cannabinoid entourages may counteract or amplify, or modify the psychoactivity of THC, via an entourage effect. The entourage effect is a proposed mechanism by which cannabis compounds other than tetrahydrocannabinol (THC) act synergistically with it to modulate the overall psychoactive effects of the plant.

In some embodiments, the entourage of cannabinoids comprises cannabichromene (CBC), cannabidiol (CBD), cannabidiolic acid (CBDA), cannabidivarin (CBDV), cannabicyclol (CBL), cannabigerol (CBG), cannabinol (CBN), delta-9-tetrahydrocannabinol (THC), delta-9-tetrahydrocannabinolic acid (THCA), and delta-9-tetrahydrocannabivarin (THCV).

The one or more cannabinoids may be present in the beverage composition in respective amounts of approximately 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104,105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 mg per serve of beverage.

In preferred embodiments, it is envisaged that the one or more cannabinoids may be present in the beverage composition in respective amounts of from about 2.5 mg to about 30 mg per serving or dose, in particular from about 5 mg to about 15 mg per serving or dose. For example, without limitation, THC and/or CBD may be present in the beverage composition in an amount of 10 mg per serving or dose. Alternatively, THC may be present in the beverage composition in an amount of 10 mg per serving or dose and CBD may be present in the beverage composition in an amount of 5 mg per serving or dose.

Consumption in these amounts is likely to result in mild relief of stress and anxiety, without inducing euphoria, and is not likely to impair coordination or perception, or lead to strong or undesirable side effects.

Cannabinoids are lipid soluble and are not dispersible in water. Accordingly, the inventors have formulated an O/W nanoemulsion to allow the desired amount of the one or more cannabinoids to be dispersed in the drinkable liquid.

The one or more cannabinoids may be readily soluble in the oil phase. The oil phase may be selected from the group comprising edible oils, short chain triglycerides, medium chain triglycerides, paraffin oil and so forth.

Suitable examples of edible oils include, but are not limited to, fish oil; vegetable oils such as peanut oil, soy bean oil, sunflower oil, safflower oil, canola oil, corn oil, avocado oil, almond oil, olive oil, cotton seed oil, coconut oil, sesame oil, chia (Salvia Hispanica L.) seed oil, wheatgerm oil, grape seed oil, rice bran oil, linseed oil, mustard oil, citrus oils, palm oil; essential oils (lemongrass, clove, tea tree, thyme, geranium, marjoram, palmarosa, rosewood, sage and mint); castor oil, hydrogenated castor oil, mineral oil, caproyl 90 and any mixtures thereof.

Short chain triglycerides (SCTs) are triglycerides comprising fatty acids of 2 to 5 carbon atoms. Suitable examples of short chain triglycerides include, but are not limited to, glycerol triacetate, glycerol tripropionate, glycerol tributyrate, glycerol trivalerate, or compounds of Formula (I);

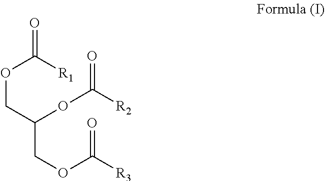

wherein R₁, R₂ and R₃ are independently selected from the group consisting of methyl, ethyl, propyl, isopropyl, cyclopropyl, n-butyl, sec-butyl, tert-butyl, cyclobutyl, ethenyl, propenyl, isopropenyl, cyclopropenyl, n-butenyl, sec-butenyl, cyclobutenyl, n-butadienyl and sec-butadienyl, including mixtures of any two or more of the aforesaid short chain triglycerides.

Medium chain triglycerides (MCTs) are triglycerides with aliphatic tails of 6 to 12 carbon atoms. Suitable examples of medium chain triglycerides include, but are not limited to, glycerol tricaproate, glycerol tricaprylate, glycerol tricaprate, glycerol trilaurate, or compounds of Formula (II);

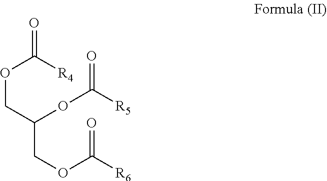

wherein R₄, R₅ and R₆ are independently selected from the group consisting of C₅₋₁₁ alkyl, C₅₋₁₁ alkenyl, C₅₋₁₁ alkadienyl, C₆₋₁₁ alkatrienyl, C₈₋₁₁ alkatetraenyl, and C₁₀₋₁₁ alkapentaenyl, wherein said alkyl, alkenyl, alkadienyl, alkatrienyl, alkatetraenyl, and alkapentaenyl substituents may each be linear, branched or cyclic, including mixtures of any two or more of the aforesaid medium chain triglycerides.

The nanoemulsion or powder may comprise a surfactant in an amount of approximately 1, 2, 3, 4, 5, 6, 7, 8, 9, 10, 11, 12, 13, 14, 15, 16, 17, 18, 19, or 20 wt %.

In preferred embodiments, the nanoemulsion comprises from 4-10 wt % surfactant. The surfactant may be a non-ionic surfactant, an ionic surfactant, or a zwiterionic surfactant, in particular a food grade surfactant, or a mixture of any of the aforementioned.

Food grade surfactants include, but are not limited to small molecule surfactants such as Tween, polysaccharides (such as maltodextrin), amphiphilic polysaccharides (gum Arabic or modified starch), phospholipids (soy, egg or dairy lecithin), amphiphilic proteins (caseinate or whey protein isolate), poly(ethylene glycols) (PEGs), Vitamin E (α-Tocopherol, α-Tocopheryl acetate, α-Tocopheryl succinate or other α-Tocopheryl derivatives), or Vitamin D (cholecalciferol, ergocalciferol, or derivatives thereof). For example, without limitation, the surfactant may be any surfactant referred to in the published review; Iva Kralova & Johan Sjöblom (2009) Surfactants Used in Food Industry: A Review, Journal of Dispersion Science and Technology, 30:9, 1363-1383, the contents of which are hereby incorporated in their entirety.

Non-ionic surfactants include a sugar ester such as sucrose monopalmitate, sorbitan monoleate, polyoxyethylene alkyl ethers (POE) or ethoxylated sorbitan esters such as Tweens 20, 60 and 80 or Spans 20, 40, 60 and 80, Solutol HS 15 (i.e. Poly(ethylene glycol)15 12-hydroxystearate), PEG-34, Medium chain triglycerides or polyoxyl 35 Castor oil.

Ionic surfactants may be a negatively charged surfactant such as sodium lauryl sulfate, diacetyl tartaric acid ester of mono-and diglycerides (DATEM), citric acid esters of mono and diglycerides (CITREM) or a positively charged surfactant such as lauric arginate.

The zwitterionic surfactant may be a phospholipid such as lecithin.

In particularly preferred embodiments, the surfactant may comprise Vitamin E (α-Tocopherol, α-Tocopheryl acetate, α-Tocopheryl succinate or other α-Tocopheryl derivatives), or Vitamin D (cholecalciferol, ergocalciferol, or derivatives thereof), to improve long-term stability in the compositions of the invention via their dual action as both surfactants, and stabilisers or antioxidants.

The beverage composition and the nanoemulsion may optionally comprise a co-surfactant to improve the long-term stability of the nanoemulsion. The co-surfactant may be a non-ionic surfactant, an ionic surfactant or a zwitterionic surfactant as described above. The co-surfactant may be, without limitation, a short-chain amine, a short-chain alcohol, a short-chain polyamine, a short-chain polyalcohol, a short-chain aminoalcohol, propylene glycol, ethylene glycol, glycerine, or a mixture of any of the aforesaid. The total concentration of surfactant and co-surfactant in the nanoemulsion preferably does not exceed 10 wt %.

The aqueous phase may be the same as or different to the drinkable liquid. For example, in embodiments where the aqueous phase is the drinkable liquid, the nanoemulsion may be prepared by mixing the oil phase containing the one or more cannabinoids, the surfactant and optionally the co-surfactant with at least a portion of the drinkable liquid. Alternatively, in embodiments where the aqueous phase is different to the drinkable liquid but miscible therewith, the nanoemulsion may be prepared by mixing the oil phase containing the one or more cannabinoids, the surfactant and optionally the co-surfactant with the aqueous phase, and then mixing the nanoemulsion with the drinkable liquid.

Alternatively, the nanoemulsion may be prepared by dissolving the particulate or powder composition of the invention in the drinkable liquid.

The beverage composition may further comprise one or more additives comprising a taste modulator (such as, without limitation, a mouth-feel modulator, or a salt, or a food acid, or a bittering agent, or a sweetening agent, or an umami agent, for example), an antioxidant, a colourant (or colouring agent), a flavourant (or flavouring agent), or a mixture thereof. The one or more additives may be soluble in the drinkable liquid. Alternatively, lipid soluble additives may be incorporated into the beverage composition by formulating the nanoemulsion to comprise one or more lipid soluble additives.

Taste modulators are substances capable of changing the flavour or mouthfeel of a food or beverage. The taste modulators may include without limitation one or more flavourants, an acid masker, cooling agent, sweet enhancer, salt enhancer, salivation-inducing substance, a substance causing a warmth or tingling feeling, and combinations thereof.

A flavourant is a substance that gives another substance flavour, by altering the characteristics of the solute, causing it to become sweet, sour tangy, and so forth. The flavourant may be a natural flavouring substance obtained from plant materials by physical, microbiological or enzymatic processes. Alternatively, the flavourant may be an artificial flavouring substance that is synthetic and which is known to impart a particular flavour to the substance to which it is added. In particular, the flavourant may be an extract, infusion, concentrate or powder (e.g. freeze-dried powder or “fruit powder”) of a fruit, or botanical source, for example. Without limitation, a native Australian fruit such as a quandong, Kakadu plum, Davidson's Plum, or finger lime.

Antioxidants are compounds capable of slowing or preventing the oxidation of other compounds. The antioxidants may include without limitation a racemic mixture of alpha.-lipoic acid, Vitamin C and its esters, Vitamin E, Vitamin E-acetate, Vitamin E derivatives, Vitamin D, Vitamin D derivatives, green tea polyphenols, green tea extract, coffee extract, chlorogenic acids, ferulic acids, caffeic acids, n-coumaric acids, theobromine, xanthine, (−)-epigallocatechin-3-gallate, (−)-epigallocatechin-3-gallate, (−)-epigallocatechin, (−)-epicatechin, carotenoids (.alpha.-, .beta.-, and .gamma.-carotene), curcuminoids such as curcumin (diferuloylmethane), desmethoxycurcumin (hydroxycirmamoyl feruloylmethane), and bis-desmethoxycurcumin (dihydroxydicinnamoyl methane), chlorophyllin and/or its salts, superoxide dismutase, glutathione peroxidase, tocotrienols, polyphenols, cysteine, and methionine.

Said beverage composition and nanoemulsions are formulated to be transparent or translucent and to be chemically stable and colour stable for a predetermined period of at least 12 months.

According to the invention, the expression “transparent or translucent emulsion” means an emulsion whose matrix allows light to pass through without causing any deviation by refraction or reflection, or causing only small deviations of the light rays at the interface of the two phases. The skilled addressee will understand that there are many routine methods in the art that may be used to measure and monitor transparency or translucency over a predetermined period of time. For example, the transparency of an emulsion can be readily evaluated with the naked eye. Alternatively it may be measured using a turbidimeter. The portable turbidimeter model Hach 2100P® may be used, for example, to measure the ranges of transparency of the emulsions according to the present invention. Emulsions are generally said to be transparent when the value measured is between 0 and 250 NTU, while they are generally said to be translucent for a value ranging from 250 to 1000 NTU. Alternatively, transparency or translucency over a predetermined period of time may be measured using Visible absorbance spectrometry. The Visible absorbance spectrometer model ThermoFisher Scientific SPECTRONIC™ 200 may be used, for example, to measure the ranges of transparency of the emulsions according to the present invention.

Similarly, the skilled addressee will understand that there are many routine methods in the art that may be used to measure and monitor chemical stability over a predetermined period of time. For example, the LCMSMS techniques employed in the examples of the present disclosure may be used to measure and monitor chemical stability over a predetermined period of time. Alternatively, other routine analytical techniques such as but limited to GCMS, HPLCMS, GCMSMS, etc, may be used to measure and monitor chemical stability over a predetermined period of time.

Similarly, the skilled addressee will understand that there are many routine methods in the art that may be used to measure and monitor colour stability over a predetermined period of time. For example, visible spectrometry and colorimetric measurements may be made using routine methods in the art, and applied accordance with accepted standards in the art. Such standards include the Commission Internationale de l'eclairage (CIE) system, involving standard-observer curves for the visible spectrum for the tristimulus values which are converted to the unreal primaries X, Y, and Z to standardise colour. Alternatively, the CIE 1976 L*a*b* (CIELAB), or HunterLab L,a,b colour scale systems may be used.

Alternatively, the Australian Standard colour scale system AS 2700 (which is hereby incorporated into the present disclosure in its entirety), as employed in the examples of the present disclosure, may be used to measure and monitor colour stability over a predetermined period of time.

AS 2700 calculates colour coordinates in the CIE 1976 (L*a*b*) colour space in accordance with AS/NZS 1580.601.2. These values are then used to calculate the CIE 1976 chroma (C*_{ab}) and hue angle (h_{ab}). The values are obtained from measurement on an integrating sphere spectrophotometer, using CIE Standard Illuminant D65 and the 10° 1964 CIE supplementary Standard Observer, with the specular component included. The wavelength range used is 400 to 700 nm with a 20 nm interval. A white cardboard backing is used behind each colour standard during measurement.

The nanoemulsions as described herein have a high colloid stability, preferably a surfactant concentration less than 10% which minimises surfactant related toxicity problems, and have a large surface area, allowing improved bio-availability of the one or more cannabinoids, thereby decreasing the period of time for onset of the psychoactive effects to the consumer.

Various embodiments of the nanoemulsion have droplets with a particle size from about 15 nm to 100 nm. It is desirable that the beverage composition may be transparent or translucent with minimal turbidity. In particular, some embodiments of the nanoemulsion may have droplets with a particle size from 20 nm to 30 nm, thereby ensuring that the beverage composition has a transparent appearance when observed by the naked eye.

Particle size distribution of the droplets in the nanoemulsion may be determined by conventional techniques as will be well understood by the person skilled in the art, such as by dynamic laser light scattering.

### Beverage Concentrate Compositions

The disclosure also provides a beverage concentrate composition which is arranged, in use, to be mixed with the drinkable liquid to prepare a beverage composition. The beverage concentrate composition may be a liquid that can be diluted by and dispersed in the drinkable liquid. Alternatively, the beverage concentrate may be a particulate or powder material that is soluble or dispersible in the drinkable liquid.

The liquid beverage concentrate composition comprises a nanoemulsion comprising one or more cannabinoids in an oil phase, a surfactant, and an aqueous phase, as described above.

The particulate material comprises a mixture of said nanoemulsion and one or more encapsulation agent(s), whereby the mixture of nanoemulsion and encapsulation agent(s) is dried to produce the particulate material. The mixture may be dried by freeze-drying (lyophilization), spray drying or electrostatic drying. The encapsulation agent may be any suitable film-forming and GRAS substance soluble in water, or a mixture of water and ethanol. For example, without limitation, the encapsulation agent may be a starch, cellulose, cellulose derivative, a polyvinyl alcohol, a gelatin, a carageenan, a hydrogel, an alginate or alginate salt, an edible polymer, a protein (such as whey protein or casein), or an ionic salt.

In a preferred embodiment, the encapsulation agent is a low-calorie encapsulation agent such as an ionic salt. In some preferred embodiments, the encapsulation agent is sodium bicarbonate.

The beverage composition may be prepared from the liquid nanoemulsion beverage concentrate composition by mixing a serving of a beverage concentrate composition as defined above in a drinkable liquid, wherein a serving comprises 1, 2, 3, 4, 5, 6,7,8, 9, 10, 11, 12, 13, 14,15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mL of nanoemulsion concentrate, per 100 mL of drinkable liquid.

The beverage composition may be prepared from the particulate or powder beverage concentrate composition by mixing a serving of a particulate or powder beverage concentrate composition as defined above in a drinkable liquid, wherein a serving comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 g of particulate or powder concentrate composition per 100 mL of drinkable liquid.

The beverage composition may be prepared from the liquid beverage concentrate composition or the particulate or powder beverage concentrate composition by mixing a serving of said beverage concentrate composition in a drinkable liquid. For example, the serving may comprise from 10 to 15 mL of said liquid beverage concentrate composition or from 1-10 g of said particulate beverage concentrate composition per 100 mL of drinkable liquid.

Advantageously, the particulate or powder concentrate compositions in accordance with the present invention enable the formulation of water soluble or dispersible cannabinoids into a dry flowable powder, allowing for unprecedented ease of handling, transportation and doseability in ingestible cannabinoid containing formulations.

Advantageously, the particulate or powder concentrate compositions in accordance with the present invention enable the addition, either prior to encapsulation, or after drying into a flowable powder form, of further excipients to assist the powder to, on contact with water, form an emulsion and produce a clear solution or suspension.

Advantageously, the particulate or powder concentrate compositions in accordance with the present invention enable the addition, either prior to encapsulation, or after drying into a flowable powder form, of further flavouring agents and/or colouring agents and/or stabilizers and/or antioxidants and/or mouth-feel enhancers and/or other additives that may improve the desirable qualities of the beverages produced therefrom.

Advantageously, the particulate or powder concentrate compositions in accordance with the present invention enable the formulation of water soluble or dispersible cannabinoids into a dry flowable powder that exhibits a high degree of stability over a period of at least 12 months.

Advantageously, the particulate or powder concentrate compositions in accordance with the present invention enable the formulation of water soluble or dispersible cannabinoids into a dry flowable powder, that upon addition to a drinkable liquid produces a colour stable and chemically stable, transparent and homogenous fluid comprising a stable emulsion that provides for a highly effective oral administration route for cannabinoids, without the need for any ethanol.

### Method of Manufacturing Beverage Composition

The beverage composition as described herein may be prepared by subjecting a mixture of the drinkable liquid, a surfactant and an oil phase comprising one or more cannabinoids to a high energy or a low energy emulsification technique for a sufficient period to produce a nanoemulsion.

The skilled addressee will be aware that various techniques exist in the art for producing emulsions, including techniques generally referred to as high energy emulsification techniques. As used herein, the term “high energy emulsification technique”, and grammatical variations thereof, will be understood to include such techniques as high-pressure homogenization, microfluidization, and ultrasonication.

The skilled addressee will also be aware that various alternative techniques exist in the art for producing emulsions, including techniques generally referred to as low enery emulsification techniques. As used herein, the term “low energy emulsification technique”, and grammatical variations thereof, will be understood to include such techniques as phase inversion emulsification methods (including transitional phase inversion and catastrophic phase inversion), and self-nanoemulsification methods.

Alternatively, the beverage composition as described herein may be prepared by:
a) subjecting a mixture of an aqueous phase, a surfactant and an oil phase comprising one or more cannabinoids to a high energy or a low energy emulsification technique for a sufficient period to produce a nanoemulsion, and
b) mixing the nanoemulsion with the drinkable liquid.

In a further alternative, the beverage composition as described herein may be prepared by:
a) subjecting a mixture of an aqueous phase, a surfactant and an oil phase to a high energy or a low energy emulsification technique for a sufficient time to produce a nanoemulsion, wherein the oil phase comprises one or more cannabinoids and the aqueous phase is miscible with a drinkable liquid;
b) adding an encapsulation agent to the nanoemulsion;
c) drying the nanoemulsion in the presence of the added encapsulation agent to provide a particulate or powder composition and;
d) mixing the particulate or powder composition with the drinkable liquid.

The nanoemulsion as described herein may be prepared by low energy or high energy techniques as will be well known to those skilled in the art. Suitable techniques include, but are not limited to, low energy techniques such as spontaneous emulsification (SE), emulsion phase inversion (EPI) or phase inversion temperature (PIT), and high energy techniques such as high pressure homogenization (HPH), high pressure valve homogenization (HPVH), microfluidification or ultrasonic homogenization.

The nanoemulsion as described herein may also be prepared by aspirating or nebulising the oil phase into the aqueous phase, wherein either or both of the oil phase and the aqueous phase contain one or more surfactants and, optionally, one or more co-surfactants.

The nanoemulsions as described herein may be readily prepared by low-energy phase inversion by gradual addition of the aqueous phase or the drinkable liquid to a mixture of the one or more cannabinoids, the oil phase, the surfactant and, optionally, the co-surfactant at ambient temperature (15° C.-30° C.) with constant stirring. The aqueous phase may be at least a portion of the drinkable liquid or an aqueous solvent.

Alternatively, the nanoemulsion may be obtained with a high-energy method that requires ultrasonic homogenization with initial pre-emulsion. For example, the initial pre-emulsification may be obtained by dispersing a predetermined amount of the mixture of the one or more cannabinoids, the oil phase, the surfactant and, optionally, the co-surfactant and the drinkable liquid at ambient temperature (15° C.-30° C.) with a mechanical stirrer operating at 300-600 rpm for 5 min-20 min. The resulting pre-emulsion may then undergo ultrasonic homogenization for at least 60 s.

Membrane emulsification is an alternative to other emulsification methods in which the dispersed phase is pressed through the pores of a porous membrane, while the continuous phase flows along the membrane surface. Droplets grow at pore openings until they are detached. Pore sizes control the size of droplets and the final nano-emulsion properties. Such methods are well documented, for example, in Nakashima, T.; Shimizu M.; Kukizaki M. (1991). “Membrane Emulsification, Operation Manual”. Industrial Research Institute of Miyazaki Prefecture, Miyazaki, Japan, the contents of which are hereby incorporated in their entirety.

Advantageously, co-surfactants may be used to tune the nanoemulsion droplet size in the nanoemulsion concentrate embodiments of the invention and/or the particulate size of the particulate or powder concentrate embodiments of the invention. In the case of the particulate or powder concentrate embodiments of the invention, even ethanol may be used as a co-surfactant be used to tune the nanoemulsion droplet size in the nanoemulsion precursor which is then spray-dried, thereby removing the ethanol from the composition but in the process, serving the purpose of controlling the particulate size of the powder by reducing the surface tension in the nanodroplet precursors to the powder.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### Examples

The following examples are to be understood as illustrative only. They should therefore not be construed as limiting the invention in any way.

### Nanoemulsion Concentrates

### Nanoemulsion Concentrate 1

Cannabis resin (6 mg) derived from cannabis flowers (via ethanol extraction of the dried flowers followed by removal of the ethanol under reduced pressure), Orange oil (0.5 ml, Sigma Aldrich), PEG 35 surfactant (0.2 ml, Sigma Aldrich), α-tocopherol (5 mg, Sigma Aldrich), α-tocopherol acetate (2.5 mg, Sigma Aldrich), and α-tocopherol succinate (2.5 mg, Sigma Aldrich) were added to MCT (5 ml, Swanson Ultra 100% Pure MCT Oil, Pharmaceutical Grade). The resultant mixture was made up to a total volume of 20 ml in water, and then subjected to ultrasonication for 20 minutes at room temperature to produce a stable nanoemulsion concentrate.

### Nanoemulsion Concentrate 2

Cannabis resin (˜30 mg) derived from cannabis flowers (via ethanol extraction of the dried flowers followed by removal of the ethanol under reduced pressure), Orange oil (2.5 ml, Sigma Aldrich), PEG 35 surfactant (0.2 ml, Sigma Aldrich), α-tocopherol (20 mg, Sigma Aldrich), α-tocopherol acetate (10 mg, Sigma Aldrich), and α-tocopherol succinate (10 mg, Sigma Aldrich) were added to MCT (25 ml, Swanson Ultra 100% Pure MCT Oil Pharmaceutical Grade), to provide a lipophilic phase.

Separately, modified starch (5 g corn starch, Woolworths), maltodextrin (3 g, Merck), Vitamin C (1 g, Merck), citric acid (0.5 g, Sigma Aldrich), sodium bicarbonate (NaHCO₃, 0.5 g, Sigma Aldrich), Magnesium Citrate (1 g, Sigma Aldrich), plant gum (3 g Guar and Xanthan gum mix, Woolworths), and soluble fibre (1 g, Benefibre, Woolworths) were combined and dissolved in 175 mL of water to provide an aqueous phase comprising antioxidants, stabilisers, encapsulating agents and encapsulating excipients.

The entire lipophilic phase was made up to a total volume of 200 ml via addition of a sufficient quantity of the aqueous phase and then the resultant mixture was subjected to ultrasonication for 20 minutes at room temperature to produce a stable nanoemulsion concentrate.

### Particulate or Powder Concentrates

200 ml of Nanoemulsion Concentrate 2 was subjected to spray drying using a Buchi-290 Mini Spray dryer (125° C. inlet temperature, 35° C. outlet temperature, 90% Aspirator) to produce 5 g of a stable, mostly free flowing powder.

The stability of the cannabinoid and Vitamin E components of the powder concentrate stored at room temperature (21-25° C.) was monitored, over a period of 570 days via Liquid Chromatography-tandem Mass Spectrometry [LCMSMS, Agilent 6590, C18, 3×50 mm, 2.7 μm (Agilent InfinityLab Poroshell 120 EC-C18) column, Electrospray Ionization detector]. The LCMSMS was calibrated for 0-1000 ng/ml cannabinoids with the same resin material used in the preparation of the concentrates. At each time period, 0.05 g of powder was dissolved in 10 ml-15 of ethanol, diluted to fall within the analytical range, and injected into the LCMSMS for analysis.

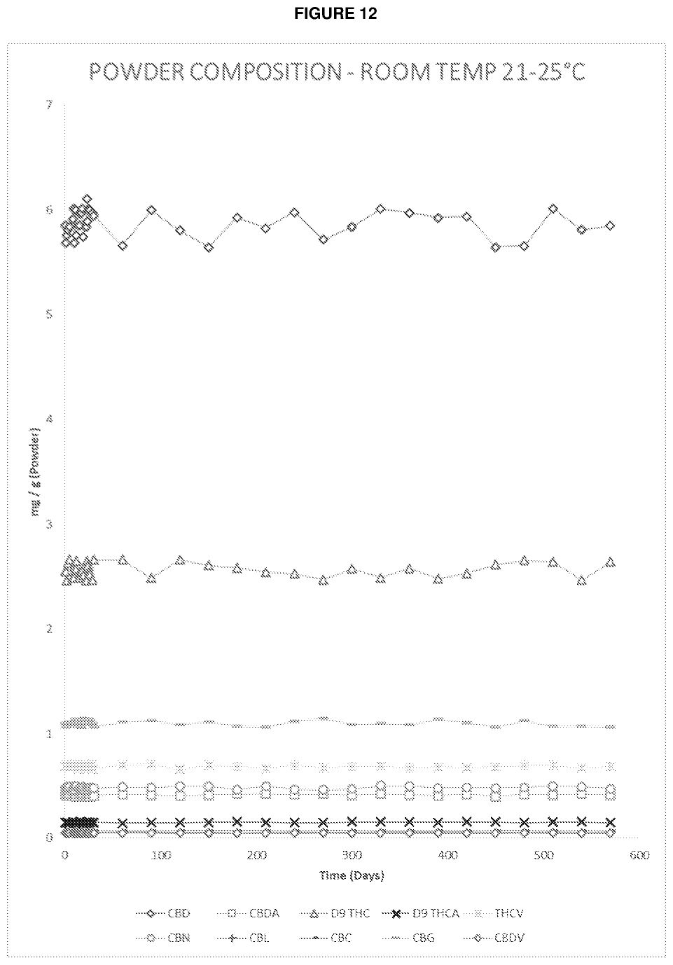

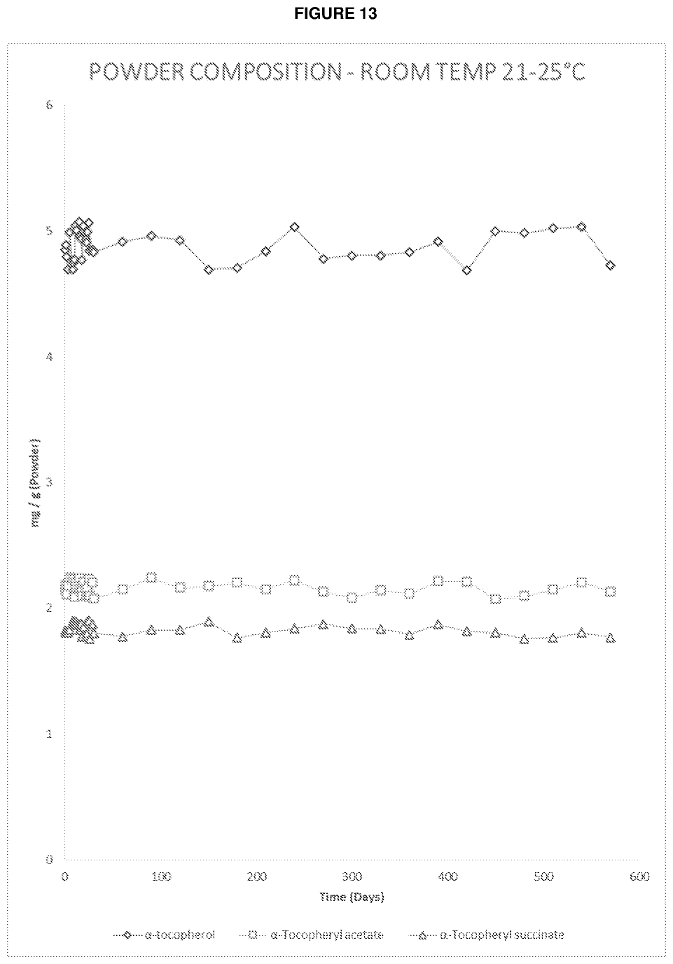

The data showed no significant degradation of CBD, THC, or any of the remaining cannabinoids in the entourage over the entire period (FIG. 12, Table 1), and no significant losses of Vitamin E over the entire period (FIG. 13, Table 2).

TABLE 1
Stability of Cannabinoids in Powder Concentrate (mg/g powder)

| Time | | | | | | | | | | |
|---|---|---|---|---|---|---|---|---|---|---|
| (Days) | CBD | CBDA | D9 THC | D9 THCA | THCV | CBN | CBL | CBC | CBG | CBDV |
| 0 | 5.85 | 0.410 | 2.55 | 0.150 | 0.680 | 0.480 | 0.050 | 0.070 | 1.1 | 0.050 |
| 1 | 5.69 | 0.412 | 2.55 | 0.150 | 0.685 | 0.471 | 0.051 | 0.072 | 1.07 | 0.051 |
| 2 | 5.76 | 0.407 | 2.46 | 0.147 | 0.699 | 0.488 | 0.049 | 0.071 | 1.08 | 0.051 |
| 3 | 5.81 | 0.406 | 2.63 | 0.150 | 0.698 | 0.474 | 0.050 | 0.073 | 1.09 | 0.048 |
| 5 | 5.84 | 0.414 | 2.66 | 0.146 | 0.676 | 0.491 | 0.052 | 0.070 | 1.11 | 0.050 |
| 8 | 5.91 | 0.415 | 2.49 | 0.156 | 0.685 | 0.469 | 0.050 | 0.071 | 1.07 | 0.051 |
| 9 | 6.01 | 0.410 | 2.49 | 0.151 | 0.693 | 0.466 | 0.050 | 0.068 | 1.09 | 0.049 |
| 10 | 5.69 | 0.418 | 2.60 | 0.148 | 0.706 | 0.499 | 0.050 | 0.071 | 1.08 | 0.050 |
| 11 | 6.00 | 0.415 | 2.58 | 0.145 | 0.708 | 0.494 | 0.052 | 0.071 | 1.14 | 0.051 |
| 12 | 5.76 | 0.396 | 2.65 | 0.152 | 0.698 | 0.496 | 0.052 | 0.069 | 1.10 | 0.051 |
| 15 | 5.85 | 0.399 | 2.49 | 0.151 | 0.684 | 0.472 | 0.048 | 0.073 | 1.08 | 0.050 |
| 16 | 5.97 | 0.427 | 2.54 | 0.157 | 0.658 | 0.483 | 0.049 | 0.073 | 1.06 | 0.049 |
| 17 | 5.97 | 0.421 | 2.54 | 0.156 | 0.681 | 0.465 | 0.049 | 0.070 | 1.06 | 0.051 |
| 18 | 6.01 | 0.418 | 2.55 | 0.149 | 0.690 | 0.470 | 0.052 | 0.072 | 1.13 | 0.051 |
| 19 | 5.74 | 0.414 | 2.51 | 0.146 | 0.705 | 0.490 | 0.052 | 0.070 | 1.14 | 0.048 |
| 22 | 5.84 | 0.419 | 2.46 | 0.150 | 0.703 | 0.486 | 0.051 | 0.071 | 1.12 | 0.049 |
| 23 | 6.10 | 0.396 | 2.65 | 0.147 | 0.687 | 0.469 | 0.051 | 0.069 | 1.13 | 0.050 |
| 24 | 5.89 | 0.403 | 2.60 | 0.150 | 0.668 | 0.465 | 0.050 | 0.068 | 1.09 | 0.051 |
| 25 | 6.01 | 0.401 | 2.55 | 0.150 | 0.689 | 0.486 | 0.050 | 0.072 | 1.13 | 0.052 |
| 26 | 6.00 | 0.397 | 2.52 | 0.150 | 0.686 | 0.486 | 0.051 | 0.071 | 1.14 | 0.050 |
| 29 | 5.97 | 0.426 | 2.47 | 0.152 | 0.706 | 0.484 | 0.048 | 0.069 | 1.10 | 0.049 |
| 30 | 5.94 | 0.405 | 2.66 | 0.151 | 0.659 | 0.476 | 0.051 | 0.072 | 1.07 | 0.051 |
| 60 | 5.66 | 0.418 | 2.66 | 0.146 | 0.702 | 0.489 | 0.050 | 0.068 | 1.1 | 0.050 |
| 90 | 6.00 | 0.413 | 2.49 | 0.147 | 0.707 | 0.483 | 0.050 | 0.068 | 1.13 | 0.050 |
| 120 | 5.80 | 0.403 | 2.66 | 0.147 | 0.658 | 0.498 | 0.048 | 0.072 | 1.09 | 0.050 |
| 150 | 5.65 | 0.412 | 2.61 | 0.153 | 0.695 | 0.500 | 0.050 | 0.072 | 1.11 | 0.051 |
| 180 | 5.93 | 0.422 | 2.59 | 0.154 | 0.685 | 0.464 | 0.049 | 0.071 | 1.07 | 0.050 |
| 210 | 5.83 | 0.427 | 2.54 | 0.151 | 0.662 | 0.501 | 0.052 | 0.072 | 1.06 | 0.049 |
| 240 | 5.98 | 0.417 | 2.53 | 0.148 | 0.700 | 0.467 | 0.049 | 0.068 | 1.12 | 0.052 |
| 270 | 5.72 | 0.424 | 2.47 | 0.148 | 0.670 | 0.464 | 0.051 | 0.072 | 1.15 | 0.051 |
| 300 | 5.84 | 0.409 | 2.57 | 0.156 | 0.685 | 0.469 | 0.050 | 0.071 | 1.09 | 0.049 |
| 330 | 6.01 | 0.424 | 2.49 | 0.156 | 0.691 | 0.502 | 0.049 | 0.069 | 1.10 | 0.052 |
| 360 | 5.97 | 0.413 | 2.58 | 0.155 | 0.668 | 0.499 | 0.050 | 0.068 | 1.09 | 0.050 |
| 390 | 5.93 | 0.407 | 2.48 | 0.153 | 0.680 | 0.479 | 0.052 | 0.068 | 1.14 | 0.049 |
| 420 | 5.94 | 0.420 | 2.53 | 0.154 | 0.671 | 0.481 | 0.050 | 0.069 | 1.11 | 0.051 |
| 450 | 5.65 | 0.398 | 2.61 | 0.155 | 0.680 | 0.480 | 0.052 | 0.073 | 1.06 | 0.049 |
| 480 | 5.66 | 0.418 | 2.65 | 0.149 | 0.695 | 0.485 | 0.051 | 0.071 | 1.12 | 0.051 |
| 510 | 6.01 | 0.411 | 2.64 | 0.154 | 0.696 | 0.499 | 0.050 | 0.069 | 1.07 | 0.049 |
| 540 | 5.81 | 0.425 | 2.47 | 0.154 | 0.664 | 0.492 | 0.048 | 0.070 | 1.07 | 0.052 |
| 570 | 5.85 | 0.408 | 2.64 | 0.148 | 0.686 | 0.471 | 0.050 | 0.069 | 1.06 | 0.048 |

TABLE 2
Stability of Vitamin Es in Powder Concentrate (mg/g powder)

| Time | | α-Tocopheryl | α-Tocopheryl |
|---|---|---|---|
| (Days) | α-Tocopherol | acetate | succinate |
| 0 | 4.85 | 2.15 | 1.82 |
| 1 | 4.89 | 2.19 | 1.81 |
| 2 | 4.79 | 2.11 | 1.81 |
| 3 | 4.69 | 2.18 | 1.81 |
| 5 | 4.99 | 2.24 | 1.83 |
| 8 | 4.69 | 2.23 | 1.90 |
| 9 | 4.75 | 2.09 | 1.89 |
| 10 | 4.77 | 2.09 | 1.87 |
| 11 | 5.04 | 2.20 | 1.89 |
| 12 | 5.01 | 2.17 | 1.87 |
| 15 | 5.07 | 2.24 | 1.87 |
| 16 | 4.96 | 2.15 | 1.83 |
| 17 | 4.94 | 2.13 | 1.88 |
| 18 | 4.77 | 2.10 | 1.77 |
| 19 | 5.04 | 2.21 | 1.85 |
| 22 | 4.94 | 2.11 | 1.78 |
| 23 | 4.91 | 2.16 | 1.80 |
| 24 | 5.00 | 2.09 | 1.90 |
| 25 | 5.06 | 2.24 | 1.90 |
| 26 | 4.84 | 2.10 | 1.76 |
| 29 | 4.85 | 2.21 | 1.87 |
| 30 | 4.83 | 2.08 | 1.80 |
| 60 | 4.91 | 2.15 | 1.77 |
| 90 | 4.96 | 2.24 | 1.83 |
| 120 | 4.93 | 2.17 | 1.83 |
| 150 | 4.69 | 2.18 | 1.89 |
| 180 | 4.70 | 2.20 | 1.77 |
| 210 | 4.84 | 2.15 | 1.80 |
| 240 | 5.03 | 2.23 | 1.84 |
| 270 | 4.78 | 2.13 | 1.87 |
| 300 | 4.81 | 2.09 | 1.84 |
| 330 | 4.81 | 2.15 | 1.83 |
| 360 | 4.83 | 2.12 | 1.79 |
| 390 | 4.91 | 2.21 | 1.87 |
| 420 | 4.69 | 2.21 | 1.82 |
| 450 | 5.00 | 2.07 | 1.80 |
| 480 | 4.98 | 2.10 | 1.76 |
| 510 | 5.02 | 2.15 | 1.76 |
| 540 | 5.03 | 2.21 | 1.80 |
| 570 | 4.72 | 2.13 | 1.77 |

### Beverages

To provide flavouring and colouring agents for the beverages, a series of fruit powders were prepared by extraction of juice from a number of botanical sources (Sunrise lime, Desert lime, Rivermint, Wattleseed, Riberry, Muntries, and Mountain Pepperberry). Each botanical juice was individually subjected to a process of encapsulation and spray drying to provide the fruit powders used as flavouring and colouring agents in the beverages of the embodiments described below. However, the skilled addressee will understand that any GRAS flavouring and/or colouring agent may be used without departing from the scope of the invention described herein.

### Beverage 1—AB White

A drinkable liquid was formulated via dissolution of Sunrise lime fruit powder (8.13 g), Desert lime fruit powder (3.13 g), Rivermint fruit powder (0.63 g) and Wattleseed fruit powder (0.63 g) in 750 ml of water.

10 ml of Nanoemulsion Concentrate 1 was dispersed in 90 ml of the drinkable liquid to provide a 100 ml serving of Beverage 1 (AB White). Separate samples of the beverage were stored at 8° C. and at room temperature (21-25° C.). The beverage remained transparent and homogeneous under observation for at least 540 days under both sets of storage conditions, and colour stability, as determined by colorimetric analysis in accordance with AS 2700 was observed for at least 390 days stored at 8° C., to 480 days stored at 21-25° C. (Table 3).

TABLE 3
Transparency and Colour Stability (AB White)

| Time | Stored at 8° C. | | Stored at 21-25° C. | |
|---|---|---|---|---|
| (Days) | Clear/Cloudy | Colour (AS 2700) | Clear/Cloudy | Colour (AS 2700) |
| 0-390 | Clear | Y31 | Clear | Y31 |
| 420-480 | Clear | Y21 | Clear | Y31 |
| 510-540 | Clear | Y11 | Clear | Y21 |
| 570 | Slightly Cloudy | Y11 | Slightly Cloudy | Y21 |

The stability of the cannabinoid and Vitamin E components of the beverage stored 8° C. and at room temperature (21-25° C.) were monitored, over a period of 570 days via Liquid Chromatography-tandem Mass Spectrometry [LCMSMS, Agilent 6590, C18, 3×50 mm, 2.7 m (Agilent InfinityLab Poroshell 120 EC-C18), Electrospray Ionization detector]. The LCMSMS was calibrated for 0-1000 ng/ml cannabinoids with the same resin material used in the preparation of the concentrates. At each time period, 100 ml of beverage was diluted with 10 ml of methanol and injected into the LCMSMS for analysis.

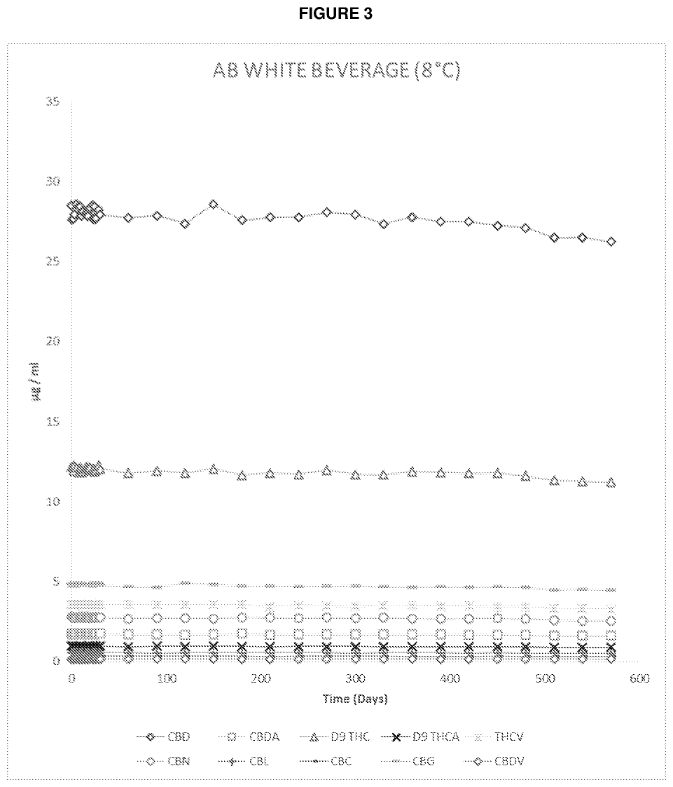

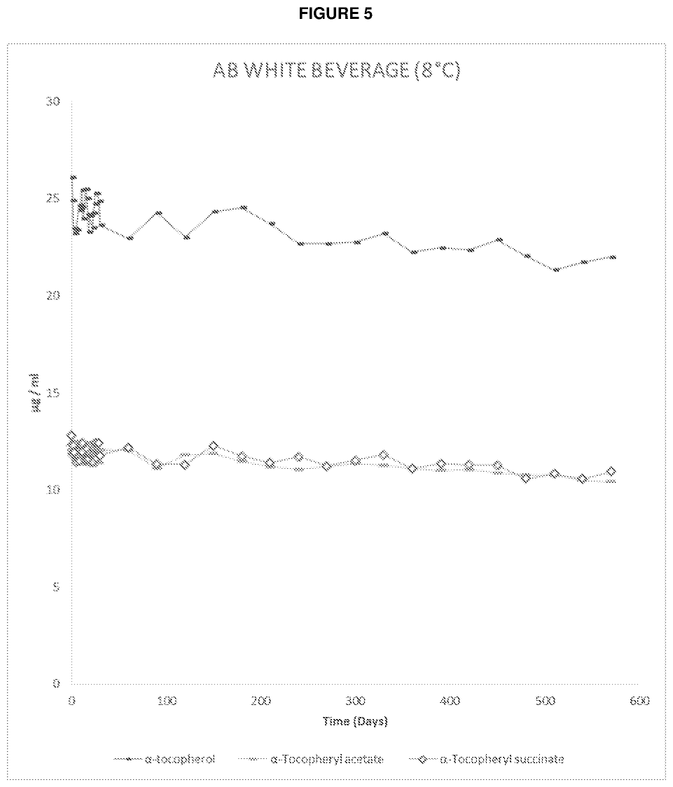

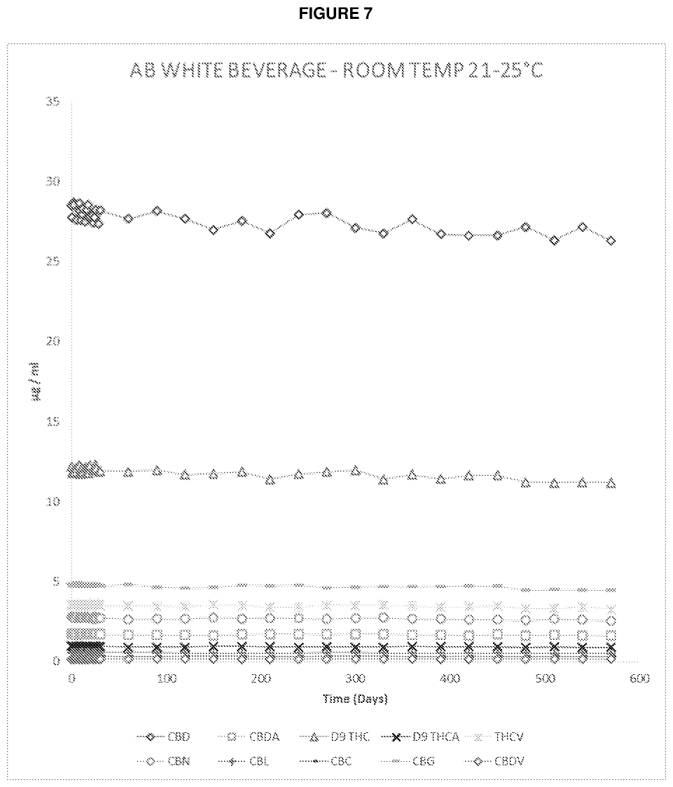

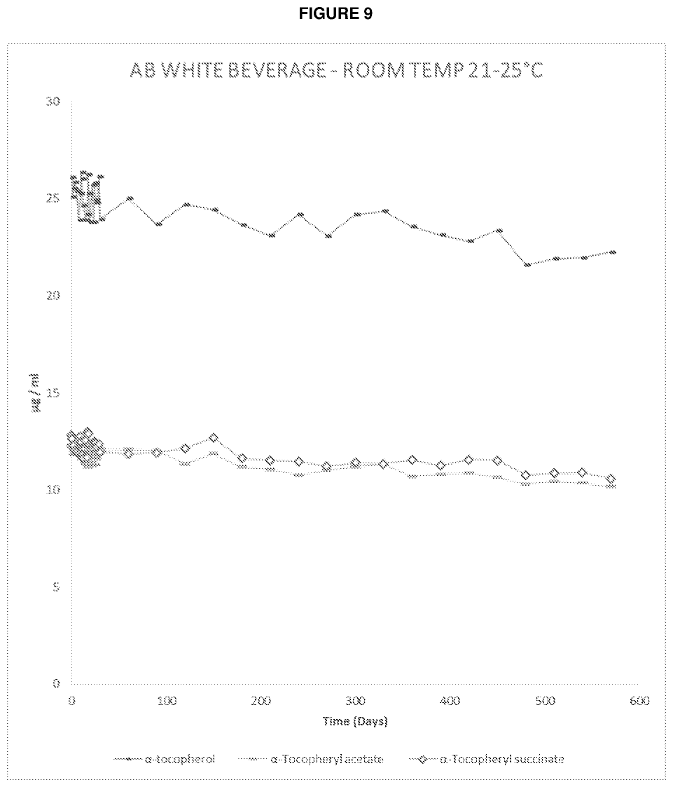

The data shows some slight degradation of CBD after 480 days (stored at 8° C.), and 390 days (stored at 21-25° C.) and a high degree of stability for THC and the remaining cannabinoids in the entourage over the entire period (FIGS. 3 and 7, Tables 4 and 5), with no significant losses of Vitamin E until after 390 days stored at 8° C. and 360 days stored at 21-25° C. (FIGS. 5 and 9, Tables 6 and 7).

TABLE 4
Stability of Cannabinoids in AB White, 8° C. (μg/mL)

| Time | | | | | | | | | | |
|---|---|---|---|---|---|---|---|---|---|---|
| (Days) | CBD | CBDA | D9 THC | D9 THCA | THCV | CBN | CBL | CBC | CBG | CBDV |
| 0 | 28.5 | 1.75 | 12.2 | 0.950 | 3.6 | 2.79 | 0.550 | 0.320 | 4.87 | 0.150 |
| 1 | 27.65 | 1.72 | 12.20 | 0.956 | 3.54 | 2.77 | 0.541 | 0.314 | 4.74 | 0.146 |
| 2 | 27.67 | 1.76 | 11.91 | 0.954 | 3.60 | 2.72 | 0.542 | 0.315 | 4.91 | 0.147 |
| 3 | 27.96 | 1.76 | 12.25 | 0.934 | 3.55 | 2.73 | 0.553 | 0.322 | 4.74 | 0.146 |
| 5 | 28.61 | 1.71 | 12.11 | 0.956 | 3.58 | 2.76 | 0.552 | 0.322 | 4.88 | 0.147 |
| 8 | 28.47 | 1.75 | 11.83 | 0.949 | 3.60 | 2.75 | 0.537 | 0.318 | 4.86 | 0.148 |
| 9 | 28.44 | 1.73 | 12.14 | 0.937 | 3.61 | 2.76 | 0.535 | 0.319 | 4.89 | 0.146 |
| 10 | 27.87 | 1.76 | 12.07 | 0.960 | 3.58 | 2.79 | 0.544 | 0.314 | 4.77 | 0.148 |
| 11 | 28.13 | 1.70 | 11.83 | 0.929 | 3.59 | 2.71 | 0.544 | 0.314 | 4.87 | 0.150 |
| 12 | 28.10 | 1.71 | 11.88 | 0.942 | 3.50 | 2.80 | 0.553 | 0.319 | 4.87 | 0.150 |
| 15 | 27.99 | 1.74 | 12.08 | 0.944 | 3.56 | 2.79 | 0.534 | 0.317 | 4.81 | 0.150 |
| 16 | 28.04 | 1.73 | 12.18 | 0.932 | 3.57 | 2.77 | 0.546 | 0.320 | 4.84 | 0.148 |
| 17 | 27.84 | 1.73 | 12.05 | 0.951 | 3.61 | 2.82 | 0.540 | 0.315 | 4.78 | 0.146 |
| 18 | 27.87 | 1.76 | 12.00 | 0.950 | 3.59 | 2.71 | 0.536 | 0.312 | 4.81 | 0.146 |
| 19 | 28.36 | 1.74 | 12.11 | 0.959 | 3.51 | 2.71 | 0.537 | 0.317 | 4.83 | 0.149 |
| 22 | 28.50 | 1.70 | 11.97 | 0.960 | 3.60 | 2.76 | 0.555 | 0.315 | 4.75 | 0.149 |
| 23 | 27.67 | 1.76 | 12.03 | 0.931 | 3.55 | 2.71 | 0.545 | 0.321 | 4.89 | 0.149 |
| 24 | 28.44 | 1.72 | 11.87 | 0.926 | 3.56 | 2.75 | 0.548 | 0.316 | 4.83 | 0.147 |
| 25 | 27.65 | 1.75 | 11.99 | 0.957 | 3.56 | 2.75 | 0.540 | 0.310 | 4.89 | 0.149 |
| 26 | 27.73 | 1.75 | 11.97 | 0.956 | 3.63 | 2.76 | 0.534 | 0.316 | 4.77 | 0.152 |
| 29 | 28.24 | 1.75 | 12.25 | 0.939 | 3.52 | 2.76 | 0.547 | 0.321 | 4.91 | 0.151 |
| 30 | 27.93 | 1.76 | 12.05 | 0.955 | 3.59 | 2.78 | 0.544 | 0.317 | 4.79 | 0.148 |
| 60 | 27.73 | 1.70 | 11.77 | 0.914 | 3.58 | 2.68 | 0.543 | 0.316 | 4.68 | 0.143 |
| 90 | 27.87 | 1.70 | 11.92 | 0.948 | 3.56 | 2.74 | 0.527 | 0.318 | 4.66 | 0.146 |
| 120 | 27.39 | 1.67 | 11.79 | 0.932 | 3.56 | 2.72 | 0.534 | 0.311 | 4.89 | 0.144 |
| 150 | 28.59 | 1.71 | 12.07 | 0.952 | 3.55 | 2.69 | 0.540 | 0.314 | 4.83 | 0.150 |
| 180 | 27.59 | 1.75 | 11.66 | 0.941 | 3.59 | 2.77 | 0.542 | 0.319 | 4.71 | 0.150 |
| 210 | 27.76 | 1.68 | 11.79 | 0.923 | 3.44 | 2.75 | 0.534 | 0.309 | 4.73 | 0.147 |
| 240 | 27.79 | 1.70 | 11.70 | 0.932 | 3.56 | 2.70 | 0.535 | 0.316 | 4.69 | 0.145 |
| 270 | 28.07 | 1.70 | 11.98 | 0.933 | 3.47 | 2.75 | 0.539 | 0.316 | 4.72 | 0.143 |
| 300 | 27.96 | 1.69 | 11.70 | 0.939 | 3.46 | 2.71 | 0.526 | 0.313 | 4.74 | 0.143 |
| 330 | 27.33 | 1.70 | 11.68 | 0.909 | 3.52 | 2.75 | 0.543 | 0.316 | 4.69 | 0.146 |
| 360 | 27.79 | 1.70 | 11.88 | 0.913 | 3.50 | 2.68 | 0.533 | 0.306 | 4.66 | 0.145 |
| 390 | 27.50 | 1.70 | 11.85 | 0.923 | 3.47 | 2.67 | 0.535 | 0.310 | 4.68 | 0.143 |
| 420 | 27.50 | 1.67 | 11.77 | 0.916 | 3.49 | 2.70 | 0.526 | 0.307 | 4.66 | 0.144 |
| 450 | 27.25 | 1.68 | 11.80 | 0.923 | 3.44 | 2.70 | 0.532 | 0.312 | 4.68 | 0.145 |
| 480 | 27.13 | 1.66 | 11.60 | 0.905 | 3.44 | 2.66 | 0.521 | 0.305 | 4.64 | 0.143 |
| 510 | 26.48 | 1.61 | 11.33 | 0.873 | 3.34 | 2.60 | 0.514 | 0.295 | 4.49 | 0.138 |
| 540 | 26.51 | 1.61 | 11.27 | 0.877 | 3.32 | 2.56 | 0.514 | 0.299 | 4.53 | 0.139 |
| 570 | 26.22 | 1.60 | 11.21 | 0.872 | 3.30 | 2.56 | 0.503 | 0.293 | 4.46 | 0.138 |

TABLE 5
Stability of Cannabinoids in AB White, 21-25° C. (μg/mL)

| Time | | | | | | | | | | |
|---|---|---|---|---|---|---|---|---|---|---|
| (Days) | CBD | CBDA | D9 THC | D9 THCA | THCV | CBN | CBL | CBC | CBG | CBDV |
| 0 | 28.5 | 1.75 | 12.2 | 0.950 | 3.6 | 2.79 | 0.550 | 0.320 | 4.87 | 0.150 |
| 1 | 27.8 | 1.76 | 11.8 | 0.960 | 3.48 | 2.79 | 0.539 | 0.313 | 4.73 | 0.149 |
| 2 | 28.7 | 1.70 | 12.0 | 0.938 | 3.58 | 2.81 | 0.540 | 0.318 | 4.72 | 0.148 |
| 3 | 28.6 | 1.71 | 11.9 | 0.920 | 3.54 | 2.73 | 0.543 | 0.311 | 4.75 | 0.149 |
| 5 | 27.6 | 1.69 | 12.1 | 0.958 | 3.55 | 2.80 | 0.530 | 0.323 | 4.89 | 0.144 |
| 8 | 28.6 | 1.77 | 12.3 | 0.937 | 3.63 | 2.71 | 0.542 | 0.319 | 4.90 | 0.145 |
| 9 | 28.2 | 1.74 | 11.8 | 0.926 | 3.55 | 2.78 | 0.535 | 0.307 | 4.79 | 0.148 |
| 10 | 27.6 | 1.73 | 11.7 | 0.935 | 3.55 | 2.77 | 0.546 | 0.312 | 4.68 | 0.147 |
| 11 | 28.0 | 1.76 | 12.1 | 0.949 | 3.59 | 2.80 | 0.552 | 0.311 | 4.74 | 0.150 |
| 12 | 28.3 | 1.74 | 11.9 | 0.935 | 3.47 | 2.75 | 0.529 | 0.316 | 4.88 | 0.152 |
| 15 | 27.5 | 1.69 | 12.1 | 0.948 | 3.48 | 2.81 | 0.548 | 0.311 | 4.87 | 0.151 |
| 16 | 28.2 | 1.75 | 12.1 | 0.921 | 3.60 | 2.75 | 0.537 | 0.321 | 4.80 | 0.145 |
| 17 | 27.8 | 1.72 | 11.9 | 0.919 | 3.51 | 2.70 | 0.554 | 0.323 | 4.80 | 0.150 |
| 18 | 28.6 | 1.75 | 11.8 | 0.928 | 3.50 | 2.78 | 0.554 | 0.313 | 4.72 | 0.151 |
| 19 | 27.8 | 1.70 | 12.3 | 0.917 | 3.59 | 2.73 | 0.552 | 0.316 | 4.68 | 0.148 |
| 22 | 27.9 | 1.70 | 11.9 | 0.947 | 3.52 | 2.69 | 0.547 | 0.322 | 4.68 | 0.148 |
| 23 | 27.4 | 1.73 | 12.0 | 0.924 | 3.57 | 2.68 | 0.530 | 0.316 | 4.85 | 0.148 |
| 24 | 27.8 | 1.70 | 12.1 | 0.927 | 3.63 | 2.79 | 0.546 | 0.308 | 4.80 | 0.145 |
| 25 | 28.2 | 1.73 | 12.3 | 0.948 | 3.54 | 2.75 | 0.532 | 0.310 | 4.86 | 0.145 |
| 26 | 28.2 | 1.75 | 12.1 | 0.921 | 3.55 | 2.69 | 0.549 | 0.308 | 4.78 | 0.149 |
| 29 | 27.4 | 1.76 | 11.9 | 0.922 | 3.62 | 2.75 | 0.552 | 0.317 | 4.76 | 0.147 |
| 30 | 28.2 | 1.71 | 11.9 | 0.934 | 3.48 | 2.72 | 0.543 | 0.317 | 4.73 | 0.151 |
| 60 | 27.7 | 1.68 | 11.9 | 0.896 | 3.50 | 2.63 | 0.544 | 0.306 | 4.83 | 0.149 |
| 90 | 28.2 | 1.67 | 12.0 | 0.892 | 3.47 | 2.66 | 0.541 | 0.305 | 4.65 | 0.143 |
| 120 | 27.7 | 1.66 | 11.7 | 0.894 | 3.45 | 2.67 | 0.516 | 0.318 | 4.59 | 0.141 |
| 150 | 27.0 | 1.64 | 11.7 | 0.934 | 3.54 | 2.77 | 0.517 | 0.312 | 4.63 | 0.146 |
| 180 | 27.5 | 1.69 | 11.9 | 0.945 | 3.52 | 2.68 | 0.527 | 0.315 | 4.79 | 0.141 |
| 210 | 26.8 | 1.73 | 11.4 | 0.913 | 3.40 | 2.74 | 0.514 | 0.307 | 4.75 | 0.142 |
| 240 | 27.9 | 1.73 | 11.7 | 0.892 | 3.41 | 2.71 | 0.515 | 0.304 | 4.79 | 0.145 |
| 270 | 28.0 | 1.71 | 11.9 | 0.905 | 3.56 | 2.67 | 0.535 | 0.316 | 4.62 | 0.148 |
| 300 | 27.1 | 1.73 | 12.0 | 0.888 | 3.52 | 2.73 | 0.532 | 0.312 | 4.67 | 0.142 |
| 330 | 26.8 | 1.72 | 11.4 | 0.894 | 3.55 | 2.75 | 0.526 | 0.312 | 4.71 | 0.144 |
| 360 | 27.6 | 1.69 | 11.7 | 0.925 | 3.49 | 2.67 | 0.526 | 0.310 | 4.69 | 0.146 |
| 390 | 26.7 | 1.67 | 11.4 | 0.907 | 3.40 | 2.66 | 0.518 | 0.303 | 4.70 | 0.146 |
| 420 | 26.6 | 1.64 | 11.7 | 0.909 | 3.46 | 2.62 | 0.523 | 0.312 | 4.72 | 0.143 |
| 450 | 26.6 | 1.70 | 11.7 | 0.921 | 3.47 | 2.61 | 0.521 | 0.310 | 4.73 | 0.144 |
| 480 | 27.2 | 1.63 | 11.2 | 0.873 | 3.30 | 2.57 | 0.510 | 0.294 | 4.46 | 0.143 |
| 510 | 26.3 | 1.66 | 11.2 | 0.903 | 3.32 | 2.66 | 0.503 | 0.293 | 4.51 | 0.139 |
| 540 | 27.2 | 1.63 | 11.2 | 0.877 | 3.44 | 2.63 | 0.510 | 0.294 | 4.48 | 0.140 |
| 570 | 26.3 | 1.61 | 11.2 | 0.877 | 3.30 | 2.56 | 0.505 | 0.295 | 4.47 | 0.138 |

TABLE 6
Stability of Vitamin Es in AB White, 8° C. (μg/mL)

| Time | | α-Tocopheryl | α-Tocopheryl |
|---|---|---|---|
| (Days) | α-Tocopherol | acetate | succinate |
| 0 | 26.1 | 12.3 | 12.8 |
| 1 | 24.90 | 12.04 | 11.83 |
| 2 | 23.46 | 12.47 | 12.31 |
| 3 | 23.21 | 11.99 | 11.94 |
| 5 | 23.39 | 11.61 | 11.44 |
| 8 | 24.65 | 12.48 | 11.48 |
| 9 | 24.42 | 11.88 | 12.08 |
| 10 | 24.57 | 12.13 | 12.28 |
| 11 | 25.45 | 11.83 | 11.94 |
| 12 | 23.97 | 12.31 | 12.41 |
| 15 | 25.48 | 12.42 | 11.56 |
| 16 | 25.02 | 11.35 | 12.09 |
| 17 | 24.17 | 11.49 | 11.60 |
| 18 | 23.28 | 11.83 | 11.59 |
| 19 | 24.12 | 12.41 | 11.89 |
| 22 | 23.51 | 11.27 | 11.81 |
| 23 | 24.27 | 11.93 | 11.46 |
| 24 | 24.72 | 12.02 | 11.75 |
| 25 | 25.28 | 12.24 | 12.41 |
| 26 | 25.25 | 12.03 | 11.64 |
| 29 | 24.87 | 11.40 | 12.43 |
| 30 | 23.64 | 12.07 | 11.78 |
| 60 | 22.96 | 12.05 | 12.18 |
| 90 | 24.27 | 11.13 | 11.33 |
| 120 | 23.01 | 11.80 | 11.31 |
| 150 | 24.32 | 11.89 | 12.29 |
| 180 | 24.54 | 11.48 | 11.74 |
| 210 | 23.71 | 11.21 | 11.40 |
| 240 | 22.68 | 11.06 | 11.70 |
| 270 | 22.68 | 11.21 | 11.24 |
| 300 | 22.76 | 11.37 | 11.53 |
| 330 | 23.21 | 11.28 | 11.81 |
| 360 | 22.25 | 11.12 | 11.10 |
| 390 | 22.45 | 11.00 | 11.34 |
| 420 | 22.35 | 11.06 | 11.30 |
| 450 | 22.88 | 10.89 | 11.28 |
| 480 | 22.05 | 10.76 | 10.58 |
| 510 | 21.32 | 10.76 | 10.83 |
| 540 | 21.72 | 10.49 | 10.58 |
| 570 | 22.00 | 10.42 | 10.93 |

TABLE 7
Stability of Vitamin Es in AB White, 21-25° C. (μg/mL)

| Time | | α-Tocopheryl | α-Tocopheryl |
|---|---|---|---|
| (Days) | α-Tocopherol | acetate | succinate |
| 0 | 26.1 | 12.3 | 12.8 |
| 1 | 25.1 | 12.2 | 12.6 |
| 2 | 25.5 | 12.3 | 12.3 |
| 3 | 25.8 | 11.8 | 12.0 |
| 5 | 25.4 | 12.0 | 12.0 |
| 8 | 23.9 | 12.4 | 11.9 |
| 9 | 25.3 | 12.2 | 12.7 |
| 10 | 26.3 | 11.8 | 12.5 |
| 11 | 26.0 | 11.7 | 11.8 |
| 12 | 24.6 | 11.6 | 11.6 |
| 15 | 23.9 | 11.4 | 12.6 |
| 16 | 24.2 | 12.3 | 11.7 |
| 17 | 26.2 | 11.2 | 13.0 |
| 18 | 25.3 | 11.9 | 12.9 |
| 19 | 23.8 | 11.4 | 12.3 |
| 22 | 25.7 | 11.3 | 12.2 |
| 23 | 23.8 | 11.9 | 12.3 |
| 24 | 25.8 | 11.8 | 12.5 |
| 25 | 25.0 | 11.3 | 12.4 |
| 26 | 24.8 | 11.6 | 12.2 |
| 29 | 26.1 | 12.1 | 12.4 |
| 30 | 23.9 | 12.1 | 12.0 |
| 60 | 25.0 | 12.1 | 11.9 |
| 90 | 23.7 | 12.0 | 11.9 |
| 120 | 24.7 | 11.4 | 12.1 |
| 150 | 24.4 | 11.9 | 12.7 |
| 180 | 23.6 | 11.2 | 11.6 |
| 210 | 23.1 | 11.1 | 11.5 |
| 240 | 24.2 | 10.8 | 11.5 |
| 270 | 23.1 | 11.0 | 11.2 |
| 300 | 24.2 | 11.2 | 11.4 |
| 330 | 24.4 | 11.3 | 11.3 |
| 360 | 23.6 | 10.7 | 11.5 |
| 390 | 23.1 | 10.8 | 11.3 |
| 420 | 22.8 | 10.9 | 11.6 |
| 450 | 23.4 | 10.6 | 11.5 |
| 480 | 21.6 | 10.3 | 10.8 |
| 510 | 21.9 | 10.4 | 10.9 |
| 540 | 22.0 | 10.4 | 10.9 |
| 570 | 22.2 | 10.2 | 10.6 |

### Beverage 2—AB Red

A drinkable liquid was formulated via dissolution of Riberry fruit powder (6.25 g), Muntries fruit powder (6.25 g), and Mountain Pepperberry fruit powder (2.5 g) in 750 ml of water.

10 ml of Nanoemulsion Concentrate 1 was dispersed in 90 ml of the drinkable liquid to provide a 100 ml serving of Beverage 2 (AB Red). Separate samples of the beverage were stored at 8° C. and at room temperature (21-25° C.). The beverage remained transparent and homogeneous for at least 390 days under both sets of storage conditions, and colour stability, as determined by colorimetric analysis in accordance with AS 2700 was observed for at least 510 days under both sets of storage conditions (Table 8).

TABLE 8
Transparency and Colour Stability (AB Red)

| Time | Stored at 8° C. | | Stored at 21-25° C. | |
|---|---|---|---|---|
| (Days) | Clear/Cloudy | Colour (AS 2700) | Clear/Cloudy | Colour (AS 2700) |
| 0-390 | Clear | R15 | Clear | R15 |
| 420-480 | Clear with trace | R15 | Clear | R15 |
| | precipitate | | | |
| 510 | Clear with trace | R13 | Clear | R14 |
| | precipitate | | | |
| 540 | Slightly cloudy | R12 | Clear | R14 |
| 570 | Slightly cloudy | R12 | Clear with trace | R14 |
| | | | precipitate | |

The stability of the cannabinoid and Vitamin E components of the beverage stored 8° C. and at room temperature (21-25° C.) were monitored, over a period of 570 days via Liquid Chromatography-tandem Mass Spectrometry [LCMSMS, AGilent 6590, C18, 3×50 mm, 2.7 μm (Agilent InfinityLab Poroshell 120 EC-C18), Electrospray Ionization detector]. The LCMSMS was calibrated for 0-50 ng/ml cannabinoids with the same resin material used in the preparation of the concentrates. At each time period, 100 μml of beverage was diluted with 10 ml of methanol and injected into the LCMSMS for analysis.

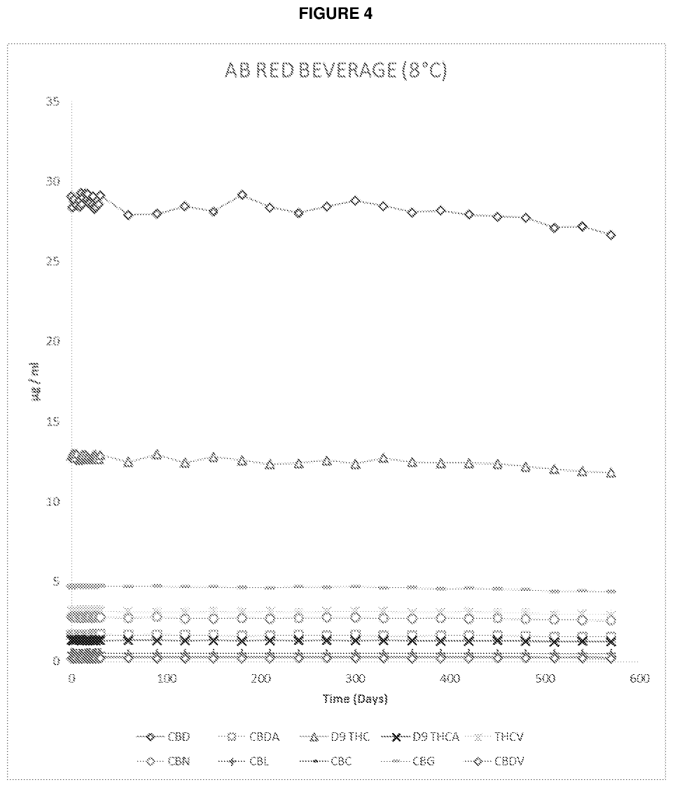

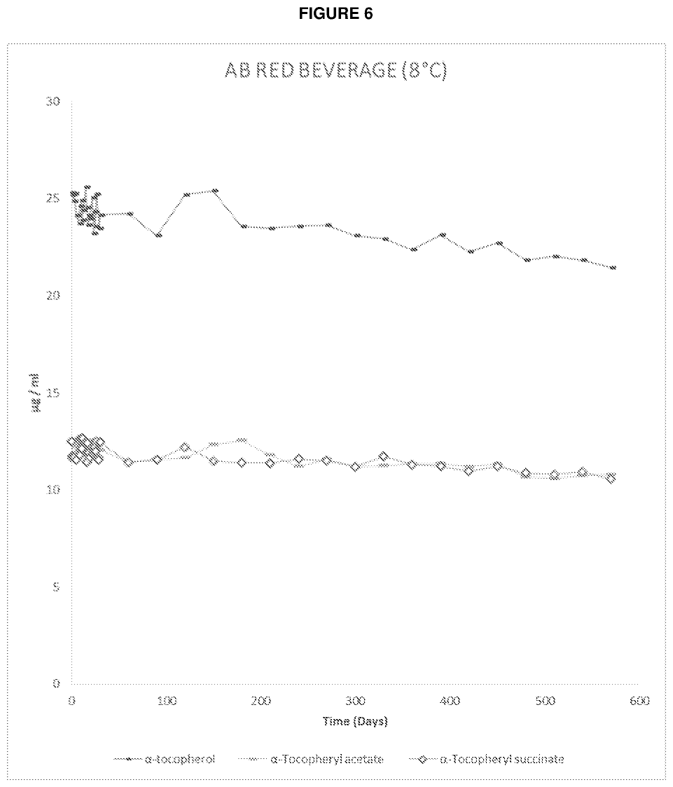

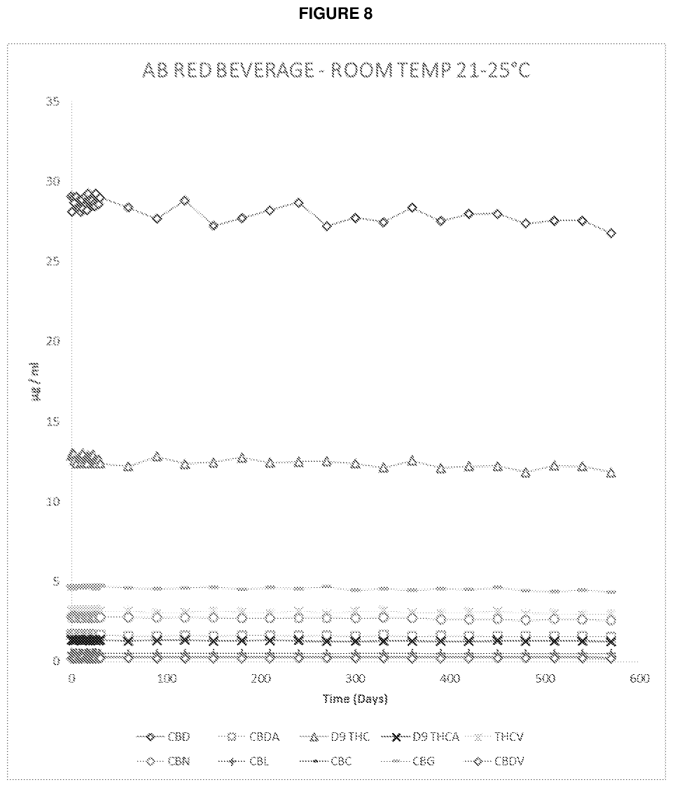

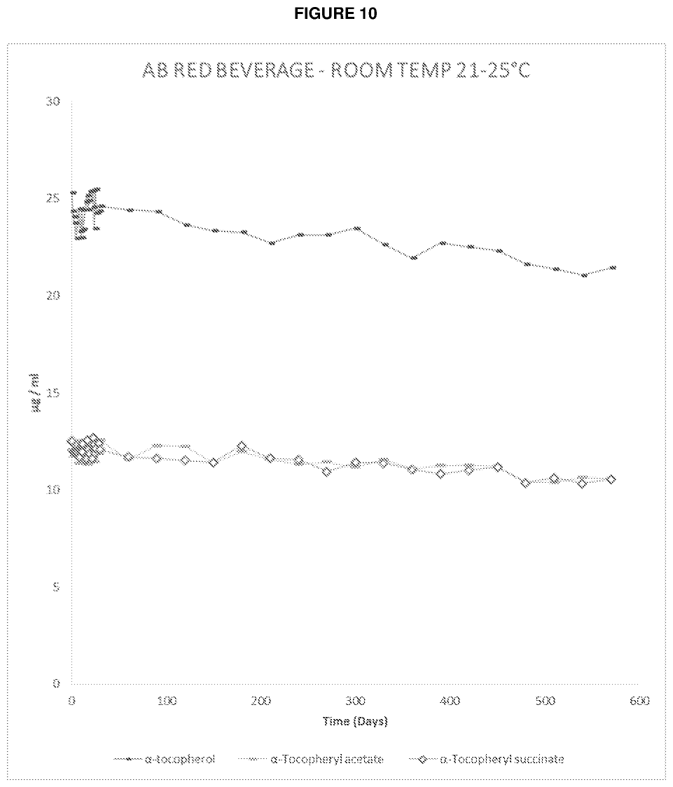

The data shows some slight degradation of CBD after 420 days (stored at 8° C.), and 390 days (stored at 21-25° C.) with a high degree of stability for THC and the remaining cannabinoids in the entourage over the entire period. (FIGS. 4 and 8, Tables 9 and 10), and no significant losses of Vitamin E until after 390 days stored at 8° C. and 360 days stored at 21-25° C. (FIGS. 6 and 10, Tables 11 and 12).

TABLE 9
Stability of Cannabinoids in AB Red, 8° C. (μg/mL)

| Time | | | | | | | | | | |
|---|---|---|---|---|---|---|---|---|---|---|
| (Days) | CBD | CBDA | D9 THC | D9 THCA | THCV | CBN | CBL | CBC | CBG | CBDV |
| 0 | 29.1 | 1.68 | 12.9 | 1.35 | 3.22 | 2.81 | 0.510 | 0.290 | 4.75 | 0.200 |
| 1 | 28.4 | 1.69 | 13.0 | 1.34 | 3.19 | 2.82 | 0.512 | 0.289 | 4.62 | 0.194 |
| 2 | 28.8 | 1.66 | 13.0 | 1.34 | 3.17 | 2.74 | 0.508 | 0.292 | 4.79 | 0.197 |
| 3 | 28.9 | 1.64 | 12.7 | 1.35 | 3.20 | 2.83 | 0.499 | 0.285 | 4.70 | 0.199 |
| 5 | 28.5 | 1.67 | 13.0 | 1.36 | 3.24 | 2.78 | 0.501 | 0.286 | 4.75 | 0.198 |
| 8 | 28.8 | 1.68 | 12.6 | 1.35 | 3.17 | 2.80 | 0.496 | 0.286 | 4.76 | 0.198 |
| 9 | 28.4 | 1.65 | 12.7 | 1.36 | 3.14 | 2.74 | 0.496 | 0.288 | 4.72 | 0.201 |
| 10 | 29.3 | 1.67 | 12.8 | 1.35 | 3.14 | 2.79 | 0.505 | 0.288 | 4.68 | 0.194 |
| 11 | 28.6 | 1.65 | 12.8 | 1.35 | 3.24 | 2.73 | 0.501 | 0.284 | 4.77 | 0.195 |
| 12 | 29.0 | 1.68 | 12.9 | 1.32 | 3.25 | 2.76 | 0.499 | 0.285 | 4.64 | 0.198 |
| 15 | 29.3 | 1.67 | 12.9 | 1.33 | 3.19 | 2.77 | 0.510 | 0.292 | 4.68 | 0.194 |
| 16 | 28.9 | 1.69 | 12.7 | 1.31 | 3.25 | 2.83 | 0.502 | 0.283 | 4.65 | 0.195 |
| 17 | 29.2 | 1.64 | 12.6 | 1.34 | 3.12 | 2.80 | 0.514 | 0.282 | 4.77 | 0.195 |
| 18 | 28.7 | 1.65 | 12.8 | 1.35 | 3.17 | 2.78 | 0.513 | 0.289 | 4.67 | 0.195 |
| 19 | 28.7 | 1.68 | 12.7 | 1.32 | 3.19 | 2.78 | 0.510 | 0.287 | 4.71 | 0.199 |
| 22 | 28.5 | 1.70 | 12.7 | 1.34 | 3.22 | 2.73 | 0.495 | 0.287 | 4.70 | 0.200 |
| 23 | 29.1 | 1.63 | 12.9 | 1.33 | 3.25 | 2.79 | 0.502 | 0.287 | 4.66 | 0.194 |
| 24 | 28.3 | 1.70 | 12.9 | 1.33 | 3.15 | 2.76 | 0.508 | 0.291 | 4.65 | 0.197 |
| 25 | 28.5 | 1.63 | 12.9 | 1.34 | 3.25 | 2.83 | 0.510 | 0.287 | 4.73 | 0.199 |
| 26 | 28.4 | 1.63 | 12.7 | 1.33 | 3.13 | 2.79 | 0.502 | 0.287 | 4.68 | 0.199 |
| 29 | 28.6 | 1.66 | 12.7 | 1.35 | 3.22 | 2.77 | 0.511 | 0.284 | 4.80 | 0.197 |
| 30 | 29.1 | 1.70 | 12.9 | 1.31 | 3.20 | 2.77 | 0.511 | 0.282 | 4.72 | 0.200 |
| 60 | 27.9 | 1.66 | 12.5 | 1.32 | 3.11 | 2.73 | 0.491 | 0.278 | 4.70 | 0.199 |
| 90 | 28.0 | 1.68 | 13.0 | 1.34 | 3.10 | 2.80 | 0.501 | 0.287 | 4.73 | 0.196 |
| 120 | 28.5 | 1.65 | 12.4 | 1.30 | 3.10 | 2.69 | 0.488 | 0.289 | 4.68 | 0.193 |
| 150 | 28.1 | 1.66 | 12.8 | 1.34 | 3.15 | 2.69 | 0.501 | 0.281 | 4.67 | 0.198 |
| 180 | 29.2 | 1.60 | 12.6 | 1.30 | 3.10 | 2.74 | 0.506 | 0.285 | 4.65 | 0.192 |
| 210 | 28.4 | 1.65 | 12.3 | 1.33 | 3.15 | 2.69 | 0.490 | 0.282 | 4.60 | 0.193 |
| 240 | 28.1 | 1.66 | 12.4 | 1.32 | 3.08 | 2.74 | 0.490 | 0.280 | 4.68 | 0.192 |
| 270 | 28.4 | 1.63 | 12.6 | 1.33 | 3.15 | 2.76 | 0.504 | 0.284 | 4.66 | 0.195 |
| 300 | 28.8 | 1.65 | 12.4 | 1.30 | 3.15 | 2.75 | 0.489 | 0.280 | 4.70 | 0.193 |
| 330 | 28.5 | 1.60 | 12.7 | 1.34 | 3.15 | 2.71 | 0.505 | 0.278 | 4.60 | 0.191 |
| 360 | 28.1 | 1.62 | 12.5 | 1.31 | 3.08 | 2.69 | 0.495 | 0.282 | 4.63 | 0.193 |
| 390 | 28.2 | 1.63 | 12.4 | 1.29 | 3.08 | 2.72 | 0.491 | 0.282 | 4.55 | 0.194 |
| 420 | 27.9 | 1.63 | 12.4 | 1.29 | 3.13 | 2.69 | 0.494 | 0.277 | 4.58 | 0.193 |
| 450 | 27.8 | 1.63 | 12.4 | 1.31 | 3.08 | 2.71 | 0.492 | 0.282 | 4.54 | 0.195 |
| 480 | 27.7 | 1.60 | 12.2 | 1.28 | 3.05 | 2.66 | 0.487 | 0.274 | 4.51 | 0.190 |
| 510 | 27.1 | 1.55 | 12.0 | 1.25 | 2.95 | 2.62 | 0.468 | 0.267 | 4.40 | 0.184 |
| 540 | 27.2 | 1.55 | 11.9 | 1.26 | 2.99 | 2.60 | 0.470 | 0.271 | 4.42 | 0.185 |
| 570 | 26.7 | 1.54 | 11.8 | 1.24 | 2.95 | 2.58 | 0.469 | 0.266 | 4.37 | 0.183 |

TABLE 10
Stability of Cannabinoids in AB Red, 21-25° C. (μg/mL)

| Time | | | | | | | | | | |
|---|---|---|---|---|---|---|---|---|---|---|
| (Days) | CBD | CBDA | D9 THC | D9 THCA | THCV | CBN | CBL | CBC | CBG | CBDV |
| 0 | 29.1 | 1.68 | 12.9 | 1.35 | 3.22 | 2.81 | 0.51 | 0.29 | 4.75 | 0.20 |
| 1 | 28.1 | 1.70 | 13.0 | 1.34 | 3.13 | 2.73 | 0.50 | 0.28 | 4.57 | 0.20 |
| 2 | 29.0 | 1.65 | 13.0 | 1.30 | 3.20 | 2.83 | 0.50 | 0.28 | 4.61 | 0.20 |
| 3 | 28.7 | 1.64 | 12.6 | 1.32 | 3.25 | 2.78 | 0.50 | 0.29 | 4.71 | 0.20 |
| 5 | 29.0 | 1.69 | 12.4 | 1.36 | 3.25 | 2.71 | 0.51 | 0.28 | 4.66 | 0.19 |
| 8 | 28.3 | 1.64 | 12.7 | 1.36 | 3.19 | 2.74 | 0.50 | 0.28 | 4.66 | 0.20 |
| 9 | 28.9 | 1.63 | 12.8 | 1.35 | 3.17 | 2.72 | 0.51 | 0.28 | 4.74 | 0.20 |
| 10 | 28.1 | 1.61 | 12.5 | 1.33 | 3.09 | 2.73 | 0.50 | 0.29 | 4.59 | 0.20 |
| 11 | 28.9 | 1.64 | 12.9 | 1.33 | 3.24 | 2.83 | 0.50 | 0.29 | 4.72 | 0.20 |
| 12 | 28.3 | 1.70 | 13.0 | 1.33 | 3.24 | 2.70 | 0.50 | 0.28 | 4.62 | 0.20 |
| 15 | 29.1 | 1.64 | 12.7 | 1.30 | 3.20 | 2.77 | 0.50 | 0.28 | 4.80 | 0.20 |
| 16 | 28.2 | 1.67 | 12.4 | 1.36 | 3.20 | 2.81 | 0.51 | 0.28 | 4.75 | 0.19 |
| 17 | 28.8 | 1.63 | 12.9 | 1.35 | 3.11 | 2.74 | 0.51 | 0.28 | 4.63 | 0.20 |
| 18 | 29.3 | 1.65 | 12.8 | 1.36 | 3.19 | 2.80 | 0.50 | 0.29 | 4.70 | 0.20 |
| 19 | 28.8 | 1.70 | 12.8 | 1.31 | 3.19 | 2.70 | 0.49 | 0.29 | 4.66 | 0.19 |
| 22 | 28.8 | 1.66 | 12.9 | 1.34 | 3.18 | 2.70 | 0.49 | 0.29 | 4.71 | 0.20 |
| 23 | 28.5 | 1.63 | 12.9 | 1.36 | 3.23 | 2.82 | 0.51 | 0.28 | 4.67 | 0.19 |
| 24 | 28.7 | 1.65 | 12.5 | 1.30 | 3.25 | 2.78 | 0.50 | 0.29 | 4.78 | 0.19 |
| 25 | 28.5 | 1.64 | 12.5 | 1.35 | 3.24 | 2.84 | 0.51 | 0.28 | 4.56 | 0.19 |
| 26 | 29.2 | 1.67 | 12.4 | 1.32 | 3.11 | 2.70 | 0.51 | 0.28 | 4.77 | 0.19 |
| 29 | 28.6 | 1.62 | 12.7 | 1.33 | 3.15 | 2.78 | 0.49 | 0.29 | 4.78 | 0.19 |
| 30 | 29.0 | 1.68 | 12.4 | 1.34 | 3.15 | 2.77 | 0.51 | 0.29 | 4.73 | 0.20 |
| 60 | 28.4 | 1.59 | 12.2 | 1.28 | 3.18 | 2.78 | 0.50 | 0.29 | 4.61 | 0.20 |
| 90 | 27.7 | 1.60 | 12.8 | 1.34 | 3.05 | 2.75 | 0.49 | 0.28 | 4.55 | 0.19 |
| 120 | 28.8 | 1.64 | 12.4 | 1.34 | 3.09 | 2.77 | 0.50 | 0.29 | 4.61 | 0.19 |
| 150 | 27.2 | 1.58 | 12.4 | 1.28 | 3.17 | 2.75 | 0.49 | 0.29 | 4.65 | 0.20 |
| 180 | 27.7 | 1.64 | 12.8 | 1.30 | 3.10 | 2.70 | 0.50 | 0.28 | 4.53 | 0.20 |
| 210 | 28.2 | 1.64 | 12.4 | 1.33 | 3.03 | 2.70 | 0.49 | 0.29 | 4.63 | 0.19 |
| 240 | 28.7 | 1.60 | 12.5 | 1.31 | 3.18 | 2.73 | 0.49 | 0.28 | 4.56 | 0.20 |
| 270 | 27.2 | 1.65 | 12.5 | 1.27 | 3.04 | 2.73 | 0.49 | 0.29 | 4.68 | 0.20 |
| 300 | 27.7 | 1.58 | 12.4 | 1.27 | 3.14 | 2.70 | 0.50 | 0.29 | 4.47 | 0.19 |
| 330 | 27.5 | 1.65 | 12.1 | 1.28 | 3.19 | 2.75 | 0.50 | 0.29 | 4.57 | 0.19 |
| 360 | 28.4 | 1.58 | 12.6 | 1.29 | 3.05 | 2.72 | 0.49 | 0.28 | 4.47 | 0.19 |
| 390 | 27.5 | 1.61 | 12.1 | 1.26 | 3.04 | 2.65 | 0.49 | 0.28 | 4.57 | 0.19 |
| 420 | 28.0 | 1.59 | 12.2 | 1.26 | 3.10 | 2.64 | 0.48 | 0.28 | 4.51 | 0.19 |
| 450 | 28.0 | 1.57 | 12.2 | 1.31 | 3.14 | 2.68 | 0.49 | 0.28 | 4.63 | 0.19 |
| 480 | 27.4 | 1.57 | 11.8 | 1.29 | 3.04 | 2.58 | 0.48 | 0.28 | 4.44 | 0.19 |
| 510 | 27.6 | 1.56 | 12.3 | 1.27 | 3.01 | 2.66 | 0.47 | 0.27 | 4.37 | 0.19 |
| 540 | 27.6 | 1.57 | 12.2 | 1.29 | 2.95 | 2.65 | 0.47 | 0.28 | 4.49 | 0.19 |
| 570 | 26.8 | 1.54 | 11.8 | 1.24 | 2.98 | 2.58 | 0.47 | 0.27 | 4.36 | 0.18 |

TABLE 11
Stability of Vitamin Es in AB Red, 8° C. (μg/mL)

| Time | | α-Tocopheryl | α-Tocopheryl |
|---|---|---|---|
| (Days) | α-Tocopherol | acetate | succinate |
| 0 | 25.3 | 12.5 | 12.5 |
| 1 | 25.2 | 11.6 | 11.7 |
| 2 | 24.9 | 11.5 | 11.7 |
| 3 | 25.3 | 11.6 | 11.7 |
| 5 | 24.2 | 12.3 | 11.5 |
| 8 | 23.7 | 12.2 | 12.6 |
| 9 | 24.6 | 12.1 | 11.8 |
| 10 | 24.9 | 12.6 | 12.6 |
| 11 | 23.9 | 11.9 | 12.1 |
| 12 | 24.4 | 12.4 | 12.7 |
| 15 | 25.6 | 12.0 | 12.5 |
| 16 | 24.5 | 11.5 | 11.5 |
| 17 | 23.6 | 11.8 | 12.4 |
| 18 | 24.1 | 12.5 | 11.9 |
| 19 | 24.0 | 12.5 | 11.7 |
| 22 | 25.0 | 12.5 | 11.9 |
| 23 | 23.2 | 11.9 | 12.0 |
| 24 | 24.3 | 12.6 | 12.2 |
| 25 | 23.6 | 12.3 | 12.0 |
| 26 | 25.2 | 11.6 | 12.5 |
| 29 | 23.5 | 11.7 | 11.6 |
| 30 | 24.2 | 12.1 | 12.4 |
| 60 | 24.2 | 11.4 | 11.4 |
| 90 | 23.1 | 11.6 | 11.5 |
| 120 | 25.2 | 11.7 | 12.2 |
| 150 | 25.4 | 12.3 | 11.5 |
| 180 | 23.6 | 12.6 | 11.4 |
| 210 | 23.5 | 11.8 | 11.4 |
| 240 | 23.6 | 11.2 | 11.6 |
| 270 | 23.6 | 11.6 | 11.5 |
| 300 | 23.1 | 11.2 | 11.2 |
| 330 | 22.9 | 11.3 | 11.7 |
| 360 | 22.4 | 11.3 | 11.3 |
| 390 | 23.1 | 11.4 | 11.2 |
| 420 | 22.3 | 11.2 | 11.0 |
| 450 | 22.7 | 11.4 | 11.2 |
| 480 | 21.8 | 10.7 | 10.9 |
| 510 | 22.0 | 10.6 | 10.8 |
| 540 | 21.8 | 10.8 | 10.9 |
| 570 | 21.5 | 10.8 | 10.6 |

TABLE 12
Stability of Vitamin Es in AB Red, 21-25° C. (μg/mL)

| Time | | α-Tocopheryl | α-Tocopheryl |
|---|---|---|---|
| (Days) | α-Tocopherol | acetate | succinate |
| 0 | 25.3 | 12.5 | 12.5 |
| 1 | 24.4 | 12.1 | 12.5 |
| 2 | 24.1 | 12.5 | 12.0 |
| 3 | 23.8 | 11.7 | 11.9 |
| 5 | 22.9 | 12.2 | 11.9 |
| 8 | 24.5 | 11.4 | 12.0 |
| 9 | 23.3 | 11.6 | 11.7 |
| 10 | 23.0 | 12.5 | 12.0 |
| 11 | 24.4 | 12.3 | 12.0 |
| 12 | 23.4 | 12.3 | 12.4 |
| 15 | 24.8 | 12.1 | 11.6 |
| 16 | 25.1 | 12.1 | 11.6 |
| 17 | 24.4 | 11.3 | 12.6 |
| 18 | 24.9 | 12.0 | 12.1 |
| 19 | 25.4 | 12.0 | 11.9 |
| 22 | 25.4 | 12.1 | 11.6 |
| 23 | 24.6 | 11.7 | 12.7 |
| 24 | 23.5 | 11.5 | 12.4 |
| 25 | 25.5 | 12.4 | 11.9 |
| 26 | 24.3 | 12.0 | 12.1 |
| 29 | 24.4 | 11.9 | 12.4 |
| 30 | 24.6 | 12.6 | 12.1 |
| 60 | 24.4 | 11.6 | 11.7 |
| 90 | 24.3 | 12.3 | 11.6 |
| 120 | 23.7 | 12.3 | 11.5 |
| 150 | 23.4 | 11.4 | 11.4 |
| 180 | 23.3 | 12.0 | 12.3 |
| 210 | 22.7 | 11.6 | 11.6 |
| 240 | 23.1 | 11.3 | 11.6 |
| 270 | 23.1 | 11.4 | 11.0 |
| 300 | 23.5 | 11.2 | 11.4 |
| 330 | 22.6 | 11.6 | 11.4 |
| 360 | 21.9 | 11.1 | 11.1 |
| 390 | 22.7 | 11.3 | 10.8 |
| 420 | 22.5 | 11.3 | 11.0 |
| 450 | 22.3 | 11.2 | 11.2 |
| 480 | 21.6 | 10.4 | 10.4 |
| 510 | 21.4 | 10.4 | 10.6 |
| 540 | 21.0 | 10.6 | 10.3 |
| 570 | 21.4 | 10.6 | 10.6 |

1. A liquid beverage concentrate composition, comprising a nanoemulsion, wherein the nanoemulsion comprises one or more cannabinoids in an oil phase, at least one surfactant, and an aqueous phase, wherein the at least one surfactant does not comprise a surfactant selected from the group consisting of; polyoxyethylenated sorbitan monooleate (Tween-80), polyoxyethylenated sorbitan monostearate (Tween-60), polyoxyethylenated sorbitan monopalmitate (Tween-40), polyoxyethylenated sorbitan monolaurate (Tween-20), sorbitan monooleate (Span 80), polyethoxylated castor oil derivatives (including Kolliphor EL, Cremophor EL and other saturated or unsaturated polyethoxylated castor oil derivatives), ethoxylated fatty acids [including poly(ethylene glycol) monooleate, and Polyethylene glycol (15)-hydroxystearate (Solutol)], polyethoxylated hydrogenated fatty acid ethers [including Polyoxyethylene (10) oleyl ether (BRIJ97)], ethoxylated monoglycerol esters, soy lecithins (including ALCOLEC F-100), sucrose fatty acid esters, sucrose palmitates, and sucrose stearates.

2. The liquid beverage concentrate composition of claim 1, wherein the one or more cannabinoids are selected from the group consisting of; anandamide, 2-arachidonoylglycerol, cannabichromene (CBC), cannabichromenic acid (CBCA), cannabichromevarin (CBCV), cannabichromevarinic acid (CBCVA), cannabidiol (CBD), cannabidiolic acid (CBDA), cannabidivarin (CBDV), cannabidivarinic acid (CBDVA), cannabielsoin (CBE), cannabicyclol (CBL), cannabinodiol (CBND), cannabigerol (CBG), cannabigerolic acid (CBGA), cannabigerovarin (CBGV), cannabigerovarinic acid (CBGVA), cannabinol (CBN), cannabinolic acid (CBNA), cannabitriol (CBT), delta-8-tetrahydrocanninol, delta-8-tetrahydrocannabinolic acid, delta-9-tetrahydrocannabinol (THC), delta-9-tetrahydrocannabinolic acid (THCA), delta-9-tetrahydrocannabivarin (THCV), delta-9-tetrahydrocannabivarinic acid (THCVA), 11-nor-9-carboxy-delta-9-tetrahydrocannabinol (THCCOOCH), 11-nor-9-carboxy-delta-8-tetrahydrocannabinol, 11-hydroxy-delta-8-tetrahydrocannabinol, and 11-hydroxy-delta-9-tetrahydrocannabinol, dimethyl heptylpentyl cannabidiol (DMHP-CBD), 6,12-dihydro-6-hydroxy-cannabidiol, (3S,4R)-7-hydroxy-.DELTA.6-tetrahydrocannabinol, (+)-4-[4-DMH-2,6-diacetoxy-phenyl]-2-carboxy-6,6-dimethylbicyclo[3.1.1]hept-2-en, (−)CBD-monomethylether, (−)CBD dimethyl ether; (−)CBD diacetate; (−)3′-acetyl-CBD monoacetate, cannabinol propyl variant (CBNV), nabilone, synthetic cannabinoids, and derivatives, analogues, stereoisomers, racemates, scalemates and pharmaceutically acceptable salts of any of the aforementioned cannabinoids.

3. The liquid beverage concentrate composition of claim 1 or claim 2, wherein the one or more cannabinoids comprise;
a) an extract of a cannabis plant; or
b) an extract of a cannabis flower; or
c) an entourage of cannabinoids; or
d) an entourage of cannabinoids comprising cannabichromene (CBC), cannabidiol (CBD), cannabidiolic acid (CBDA), cannabidivarin (CBDV), cannabicyclol (CBL), cannabigerol (CBG), cannabinol (CBN), delta-9-tetrahydrocannabinol (THC), delta-9-tetrahydrocannabinolic acid (THCA), and delta-9-tetrahydrocannabivarin (THCV); or
e) THC; or
f) CBD; or
g) THC and CBD.

4. The liquid beverage concentrate composition of any one of claims 1 to 3 wherein the nanoemulsion comprises from 4-10 wt % surfactant.

5. The liquid beverage concentrate composition of any one of claims 1 to 4 wherein the nanoemulsion further comprises a co-surfactant.

6. The liquid beverage concentrate composition of any one of claims 1 to 5 wherein the oil phase comprises an oil selected from the group consisting of;
an edible oil, a paraffin oil, an essential oil, orange oil;
a short chain triglyceride, selected from the group consisting of; glycerol triacetate, glycerol tripropionate, glycerol tributyrate, glycerol trivalerate, or a compound of Formula (I); 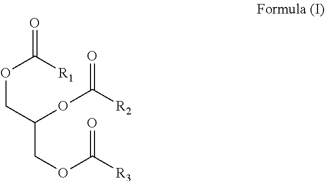
wherein R₁, R₂ and R₃ are independently selected from the group consisting of methyl, ethyl, propyl, isopropyl, cyclopropyl, n-butyl, sec-butyl, tert-butyl, cyclobutyl, ethenyl, propenyl, isopropenyl, cyclopropenyl, n-butenyl, sec-butenyl, cyclobutenyl, n-butadienyl and sec-butadienyl, including mixtures of any two or more of the aforesaid short chain triglycerides; or
a medium chain triglyceride, selected from the group consisting of; glycerol tricaproate, glycerol tricaprylate, glycerol tricaprate, glycerol trilaurate, or a compound of Formula (II); 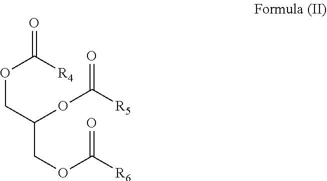
wherein R₄, R₅ and R₆ are independently selected from the group consisting of C₅₋₁₁ alkyl, C₅₋₁₁ alkenyl, C₅₋₁₁ alkadienyl, C₆₋₁₁ alkatrienyl, C₈₋₁₁ alkatetraenyl, and C₁₀₋₁₁ alkapentaenyl, wherein said alkyl, alkenyl, alkadienyl, alkatrienyl, alkatetraenyl, and alkapentaenyl substituents may each be linear, branched or cyclic, including mixtures of any two or more of the aforesaid medium chain triglycerides;
including mixtures of any of the aforesaid oils.

7. The liquid beverage concentrate composition of any one of claims 1 to 6 further comprising one or more additives selected from the group consisting of a taste modulator, an antioxidant, a colourant, a micronutrient, or a mixture of any of the aforementioned additives; wherein one or more additives are soluble in the aqueous phase, and/or one or more additives are soluble in the oil phase.

8. The liquid beverage concentrate composition of any one of claims 1 to 7, further comprising one or more encapsulation agents or encapsulation excipients, soluble in the aqueous phase, preferably wherein the one or more encapsulation agents or encapsulation excipients is selected from the group consisting of a starch, a polysaccharide, cellulose, a cellulose derivative, a polyvinyl alcohol, a gelatin, a carageenan, a hydrogel, an alginate, an alginate salt, an edible polymer, a protein, whey protein, casein, an ionic salt and sodium bicarbonate.

9. The liquid beverage concentrate composition of any one of claims 1 to 8 comprising;
a) an extract of a cannabis plant, preferably cannabis resin;
b) an essential oil, preferably orange oil;
c) a medium chain triglyceride, preferably glycerol tricaproate;
d) a poly(ethylene glycol), preferably PEG 35;
e) a combination of α-tocopherol, α-tocopherol acetate and α-tocopherol succinate, preferably in a ratio of 2:1:1; and
f) water.

10. The liquid beverage concentrate composition of claim 9, further comprising;
g) a modified starch, preferably corn starch;
h) a polysaccharide, preferably maltodextrin;
i) an antioxidant, preferably vitamin C;
j) a food acid, preferably citric acid;
k) an inorganic salt, preferably sodium bicarbonate;
l) an organic slat, preferably magnesium citrate;
m) a plant gum, preferably guar gum and/or xanthan gum; and optionally
n) a soluble fibre.

11. A process for the preparation of a particulate or powder beverage concentrate composition comprising one or more cannabinoids, said process comprising drying a liquid beverage concentrate composition in accordance with any one of claims 8 to 10, preferably wherein the drying comprises spray drying.

12. A particulate or powder beverage concentrate composition which is soluble or dispersible in a drinkable liquid and contains one or more cannabinoids, wherein said particulate or powder beverage concentrate composition comprises a mixture of the components of the liquid beverage concentrate composition in accordance with any one of claims 1 to 10, except for the aqueous phase component.

13. The particulate or powder beverage concentrate composition, obtained by the process of claim 11.

14. The particulate or powder beverage concentrate composition of claim 12 or claim 13, further comprising one or more additives selected from the group consisting of a taste modulator, an antioxidant, a colourant, a micronutrient, or a mixture of any of the aforementioned additives, wherein the one or more additives is in a particulate or powdered form that is water soluble or dispersible.

15. A beverage, comprising the liquid beverage concentrate composition of any one of claims 1 to 10, diluted or dispersed in a drinkable liquid, or the particulate or powder beverage concentrate composition of any one of claims 12 to 14, dissolved or dispersed in a drinkable liquid.

16. The beverage of claim 15, wherein;
a) optionally, a serving of the beverage is a 100 ml serving; and
b) the one or more cannabinoids are present in the beverage in respective amounts of from about 1 mg to about 500 mg per serving of the beverage; or
c) the one or more cannabinoids are present in the beverage in respective amounts of from about 2.5 mg to about 30 mg, per serving of the beverage; or
d)THC is present in the beverage in an amount of 10 mg and CBD is present in the beverage in an amount of 5 mg, per serving of the beverage; or
e) the one or more cannabinoids are present in the beverage in respective amounts of from about 10 mg/L to about 5000 mg/L; or
f) the one or more cannabinoids are present in the beverage in respective amounts of from about 25 mg/L to about 300 mg/L; or
g) THC is present in the beverage in an amount of 50 to 150 mg/L and CBD is present in the beverage in an amount of 25 to 75 mg/L; or
h)THC is present in the beverage in an amount of 100 mg/L and CBD is present in the beverage in an amount of 50 mg/L; and/or
i) wherein the drinkable liquid comprises water, carbonated water, flavoured carbonated water, juice of one or more fruits and/or vegetables, or a mixture thereof.

17. The liquid beverage concentrate composition of any one of claims 1 to 10, or the beverage of claim 15 or claim 16, which exhibits transparency or translucency stability, and/or chemical stability, and/or colour stability, for a period of at least 12 months; or the particulate or powder beverage concentrate composition of any one of claims 12 to 14, which exhibits chemical stability, and/or colour stability, for a period of at least 12 months.

18. The liquid beverage concentrate composition, or the beverage of claim 17, wherein transparency or translucency stability, is determined by evaluating, for a period of at least 12 months, transparency or translucency with the naked eye, and/or by measuring for a period of at least 12 months, transparency or translucency using a turbidimeter, and/or by measuring for a period of at least 12 months, transparency or translucency via visible absorbance spectrometry.

19. The liquid beverage concentrate composition, or the beverage, or the particulate or powder beverage concentrate composition of claim 17, wherein chemical stability, is determined by analysing, for a period of at least 12 months, the degradation of one or more cannabinoid compounds via LCMSMS, GCMS, HPLCMS, or GCMSMS.

20. The liquid beverage concentrate composition, or the beverage, or the particulate or powder beverage concentrate composition of claim 17, wherein colour stability, is determined by evaluating, for a period of at least 12 months, consistently maintained identity of colour in accordance with CIE 1976 L*a*b* (CIELAB), and/or HunterLab L,a,b and/or AS 2700 colour scale systems.
